SEQUENCE LISTING (92 sequences)

SEQ ID NO: 1 - DNA, 4361 bp, Homo sapiens
  misc_feature 1..4361: note = Homo sapiens peptidyl arginine deiminase 2 (PADI2), mRNA
  source 1..4361: mol_type = other DNA
aggctgctggagaaggcgcacctgctgcaggtgctcccggccgccccggaccagcgagcgcgggcactgcggcggggaggatgctgcgcgagcggaccgtgcggctgcagtacgggagccgcgtggaggcggtgtacgtgctgggcacctacctctggaccgatgtctacagcgcggccccagccggggcccaaaccttcagcctgaagcactcggaacacgtgtgggtggaggtggtgcgtgatggggaggctgaggaggtggccaccaatggcaagcagcgctggcttctctcgcccagcaccaccctgcgggtcaccatgagccaggcgagcaccgaggccagcagtgacaaggtcaccgtcaactactatgacgaggaagggagcattcccatcgaccaggcggggctcttcctcacagccattgagatctccctggatgtggacgcagaccgggatggtgtggtggagaagaacaacccaaagaaggcatcctggacctggggccccgagggccagggggccatcctgctggtgaactgtgaccgagagacaccctggttgcccaaggaggactgccgtgatgagaaggtctacagcaaggaagatctcaaggacatgtcccagatgatcctgcggaccaaaggccccgaccgcctccccgccggatacgagatagttctgtacatttccatgtcagactcagacaaagtgggcgtgttctacgtggagaacccgttcttcggccaacgctatatccacatcctgggccggcggaagctctaccatgtggtcaagtacacgggtggctccgcggagctgctgttcttcgtggaaggcctctgtttccccgacgagggcttctcaggcctggtctccatccatgtcagcctgctggagtacatggcccaggacattcccctgactcccatcttcacggacaccgtgatattccggattgctccgtggatcatgacccccaacatcctgcctcccgtgtcggtgtttgtgtgctgcatgaaggataattacctgttcctgaaagaggtgaagaaccttgtggagaaaaccaactgtgagctgaaggtctgcttccagtacctaaaccgaggcgatcgctggatccaggatgaaattgagtttggctacatcgaggccccccataaaggcttccccgtggtgctggactctccccgagatggaaacctaaaggacttccctgtgaaggagctcctgggcccagattttggctacgtgacccgggagcccctctttgagtctgtcaccagccttgactcatttggaaacctggaggtcagtcccccagtgaccgtgaacggcaagacatacccgcttggccgcatcctcatcgggagcagctttcctctgtctggtggtcggaggatgaccaaggtggtgcgtgacttcctgaaggcccagcaggtgcaggcgcccgtggagctctactcagactggctgactgtgggccacgtggatgagttcatgtcctttgtccccatccccggcacaaagaaattcctgctactcatggccagcacctcggcctgctacaagctcttccgagagaagcagaaggacggccatggagaggccatcatgttcaaaggcttgggtgggatgagcagcaagcgaatcaccatcaacaagattctgtccaacgagagccttgtgcaggagaacctgtacttccagcgctgcctagactggaaccgtgacatcctcaagaaggagctgggactgacagagcaggacatcattgacctgcccgctctgttcaagatggacgaggaccaccgtgccagagccttcttcccaaacatggtgaacatgatcgtgctggacaaggacctgggcatccccaagccattcgggccacaggttgaggaggaatgctgcctggagatgcacgtgcgtggcctcctggagcccctgggcctcgaatgcaccttcatcgacgacatttctgcctaccacaaatttctgggggaagtccactgtggcaccaacgtccgcaggaagcccttcaccttcaagtggtggcacatggtgccctgacctgccaggggccctggcgtttgcctccttcgcttagttctccagaccctccctcacacgcccagagccttctgctgacatggactggacagccccgctgggagacctttgggacgtggggtggaatttggggtatctgtgccttgccctccctgagaggggcctcagtgtcctctgaagccatccccagtgagcctcgactctgtccctgctgaaaatagctgggccagtgtctctgtagccctgacataaggaacagaacacaacaaaacacagcaaaccatgtgcccaaactgctccccaaagaattttgagtctctaatctgacactgaatgaggggagaagggaaggagattctgggattgccagttcttccagcagccatgctctgaaaatcaaggtagaatccatggaaagggaccccaggaccccgggaccctagacgtatcttgaactgccatcgtcatttcaaatacatctccctcagggtttccaggtggccacccccaattattcattccttaccaacctctcaaatcctcttggctttctctctgcagtgtggacactgttggctagtcctccccactccctgagggtccagtaagttagcttagaaccttcctggaaacatttcatctgagcaggtttccccacgtgtgggatgctccttttgcctcatctgtctcagggatgcaggctcccccgcatgcatggggatttctccccagaccagcatacttgtgacctgagagttcaatgcgtaaagatgcccctggtcagccatatccatcttctcttgcctggtccttgattctctggccgctccctgaccttcctccttccactgccttgactttcttcctttttattcctggtgccatctgtccaggcagctagacaagaacttgttcgccagcagccagattcaggccttcccaggggcataataagtgaccagcccctcctctccggacatcagatccaacacataaggaccctggcctaccctccagcccaacagccagttctgggtcagctgccaacttaggggtggtttgattatcccattgaaattcaccagtgcctttgccaaagaccctctcatttggacatacccagattcattccctggctccaactgaaaagactcagtttcaatcgttaaaagttcctttagggccagaagaataaatgaattataatcccattttgaagaaccgatttataaccaatgaaaaggttataatgtaatttatattcttggaggaacaagattttcatttgggattatttccttcaaccattcaacaaacatttgttgtatgccactaagcgccaggcacggcgttgggctctgcaaacacagtggttagtagcagtctggacctggtccctactggcatggaacccatcactccccaacatgcaaagcccacatttaaaggccagcctctgccccttcagtgatgcgctctttagaaatgccagtccactatattcagaaatccgcagggcacaaaacttccagcaagtcactgttgtggtgaaatgggcagtgggggtggggggtcttctttaaacaggcccccttcccatctacctagccagtacccatccaatgagtccccagagcctccagaagctgttgtctcctctctggggacagcagctcctgcctttggaggccaaagccccagatctctccagccccagagctgaaaacaccaagtgcctatttgagggtgtctgtctggagacttagagtttgtcatgtgtgtgtgtgtgtttggttaatgtgggtttatgggttttctttcttttttttctttttttttttagtctacattagggggaagtgagcgcctcccatgtgcagacagtgtgtctttatagatttttctaaggctttccccaatgatgtcggtaatttctgatgtttctgaagttcccaggactcacacacccgttcccatctcacttgcccacccagtgtgacaaccctcggtgtggatatacccccgtggactcatggctcttccccacccccactttctataaatgtaggcctagaatacgcttctctgttgcaaaactcagctaagttcctgcttccaccttgatgttgaaatatcttatgtaagagggcaggggatgtcgtgaagatggcaagaagaacacagtttcaaatttctggaaaagagcctgtggtggagatctaaagatgtttagggaagagctcgactaaagaacaatgaaataaatggtccaaggggaagtca

SEQ ID NO: 2 - protein, 665 aa, Homo sapiens
  REGION 1..665: note = protein-arginine deiminase type-2 [Homo sapiens]
MLRERTVRLQYGSRVEAVYVLGTYLWTDVYSAAPAGAQTFSLKHSEHVWVEVVRDGEAEEVATNGKQRWLLSPSTTLRVTMSQASTEASSDKVTVNYYDEEGSIPIDQAGLFLTAIEISLDVDADRDGVVEKNNPKKASWTWGPEGQGAILLVNCDRETPWLPKEDCRDEKVYSKEDLKDMSQMILRTKGPDRLPAGYEIVLYISMSDSDKVGVFYVENPFFGQRYIHILGRRKLYHVVKYTGGSAELLFFVEGLCFPDEGFSGLVSIHVSLLEYMAQDIPLTPIFTDTVIFRIAPWIMTPNILPPVSVFVCCMKDNYLFLKEVKNLVEKTNCELKVCFQYLNRGDRWIQDEIEFGYIEAPHKGFPVVLDSPRDGNLKDFPVKELLGPDFGYVTREPLFESVTSLDSFGNLEVSPPVTVNGKTYPLGRILIGSSFPLSGGRRMTKVVRDFLKAQQVQAPVELYSDWLTVGHVDEFMSFVPIPGTKKFLLLMASTSACYKLFREKQKDGHGEAIMFKGLGGMSSKRITINKILSNESLVQENLYFQRCLDWNRDILKKELGLTEQDIIDLPALFKMDEDHRARAFFPNMVNMIVLDKDLGIPKPFGPQVEEECCLEMHVRGLLEPLGLECTFIDDISAYHKFLGEVHCGTNVRRKPFTFKWWHMVP

SEQ ID NO: 3 - DNA, 3459 bp, Homo sapiens
  misc_feature 1..3459: note = PREDICTED: Homo sapiens peptidyl arginine deiminase 2 (PADI2), transcript variant X2, mRNA
  source 1..3459: mol_type = unassigned DNA
agcagctctgcagatggggattcttctgtcagctcatatctgcacatgtgcacaaagacataaacatacaagtgcactacagtgctgcttcttgtagtagcaaaagattgcaaagcaacctaaatgtccatctgtggaggactgagtaacggccttcccggaagttctgtgcaaccgctaaaaaggatgaagccagtctctcaatatggatacaggatgtgcttcaaggatgaaattgagtttggctacatcgaggccccccataaaggcttccccgtggtgctggactctccccgagatggaaacctaaaggacttccctgtgaaggagctcctgggcccagattttggctacgtgacccgggagcccctctttgagtctgtcaccagccttgactcatttggaaacctggaggtcagtcccccagtgaccgtgaacggcaagacatacccgcttggccgcatcctcatcgggagcagctttcctctgtctggtggtcggaggatgaccaaggtggtgcgtgacttcctgaaggcccagcaggtgcaggcgcccgtggagctctactcagactggctgactgtgggccacgtggatgagttcatgtcctttgtccccatccccggcacaaagaaattcctgctactcatggccagcacctcggcctgctacaagctcttccgagagaagcagaaggacggccatggagaggccatcatgttcaaaggcttgggtgggatgagcagcaagcgaatcaccatcaacaagattctgtccaacgagagccttgtgcaggagaacctgtacttccagcgctgcctagactggaaccgtgacatcctcaagaaggagctgggactgacagagcaggacatcattgacctgcccgctctgttcaagatggacgaggaccaccgtgccagagccttcttcccaaacatggtgaacatgatcgtgctggacaaggacctgggcatccccaagccattcgggccacaggttgaggaggaatgctgcctggagatgcacgtgcgtggcctcctggagcccctgggcctcgaatgcaccttcatcgacgacatttctgcctaccacaaatttctgggggaagtccactgtggcaccaacgtccgcaggaagcccttcaccttcaagtggtggcacatggtgccctgacctgccaggggccctggcgtttgcctccttcgcttagttctccagaccctccctcacacgcccagagccttctgctgacatggactggacagccccgctgggagacctttgggacgtggggtggaatttggggtatctgtgccttgccctccctgagaggggcctcagtgtcctctgaagccatccccagtgagcctcgactctgtccctgctgaaaatagctgggccagtgtctctgtagccctgacataaggaacagaacacaacaaaacacagcaaaccatgtgcccaaactgctccccaaagaattttgagtctctaatctgacactgaatgaggggagaagggaaggagattctgggattgccagttcttccagcagccatgctctgaaaatcaaggtagaatccatggaaagggaccccaggaccccgggaccctagacgtatcttgaactgccatcgtcatttcaaatacatctccctcagggtttccaggtggccacccccaattattcattccttaccaacctctcaaatcctcttggctttctctctgcagtgtggacactgttggctagtcctccccactccctgagggtccagtaagttagcttagaaccttcctggaaacatttcatctgagcaggtttccccacgtgtgggatgctccttttgcctcatctgtctcagggatgcaggctcccccgcatgcatggggatttctccccagaccagcatacttgtgacctgagagttcaatgcgtaaagatgcccctggtcagccatatccatcttctcttgcctggtccttgattctctggccgctccctgaccttcctccttccactgccttgactttcttcctttttattcctggtgccatctgtccaggcagctagacaagaacttgttcgccagcagccagattcaggccttcccaggggcataataagtgaccagcccctcctctccggacatcagatccaacacataaggaccctggcctaccctccagcccaacagccagttctgggtcagctgccaacttaggggtggtttgattatcccattgaaattcaccagtgcctttgccaaagaccctctcatttggacatacccagattcattccctggctccaactgaaaagactcagtttcaatcgttaaaagttcctttagggccagaagaataaatgaattataatcccattttgaagaaccgatttataaccaatgaaaaggttataatgtaatttatattcttggaggaacaagattttcatttgggattatttccttcaaccattcaacaaacatttgttgtatgccactaagcgccaggcacggcgttgggctctgcaaacacagtggttagtagcagtctggacctggtccctactggcatggaacccatcactccccaacatgcaaagcccacatttaaaggccagcctctgccccttcagtgatgcgctctttagaaatgccagtccactatattcagaaatccgcagggcacaaaacttccagcaagtcactgttgtggtgaaatgggcagtgggggtggggggtcttctttaaacaggcccccttcccatctacctagccagtacccatccaatgagtccccagagcctccagaagctgttgtctcctctctggggacagcagctcctgcctttggaggccaaagccccagatctctccagccccagagctgaaaacaccaagtgcctatttgagggtgtctgtctggagacttagagtttgtcatgtgtgtgtgtgtgtttggttaatgtgggtttatgggttttctttcttttttttctttttttttttagtctacattagggggaagtgagcgcctcccatgtgcagacagtgtgtctttatagatttttctaaggctttccccaatgatgtcggtaatttctgatgtttctgaagttcccaggactcacacacccgttcccatctcacttgcccacccagtgtgacaaccctcggtgtggatatacccccgtggactcatggctcttccccacccccactttctataaatgtaggcctagaatacgcttctctgttgcaaaactcagctaagttcctgcttccaccttgatgttgaaatatcttatgtaagagggcaggggatgtcgtgaagatggcaagaagaacacagtttcaaatttctggaaaagagcctgtggtggagatctaaagatgtttagggaagagctcgactaaagaacaatgaaataaatggtccaaggggaagtca

SEQ ID NO: 4 - protein, 350 aa, Homo sapiens
  REGION 1..350: note = protein-arginine deiminase type-2 isoform X1 [Homo sapiens]
MSICGGLSNGLPGSSVQPLKRMKPVSQYGYRMCFKDEIEFGYIEAPHKGFPVVLDSPRDGNLKDFPVKELLGPDFGYVTREPLFESVTSLDSFGNLEVSPPVTVNGKTYPLGRILIGSSFPLSGGRRMTKVVRDFLKAQQVQAPVELYSDWLTVGHVDEFMSFVPIPGTKKFLLLMASTSACYKLFREKQKDGHGEAIMFKGLGGMSSKRITINKILSNESLVQENLYFQRCLDWNRDILKKELGLTEQDIIDLPALFKMDEDHRARAFFPNMVNMIVLDKDLGIPKPFGPQVEEECCLEMHVRGLLEPLGLECTFIDDISAYHKFLGEVHCGTNVRRKPFTFKWWHMVP

SEQ ID NO: 5 - DNA, 3189 bp, Homo sapiens
  misc_feature 1..3189: note = Homo sapiens peptidyl arginine deiminase 3 (PADI3), mRNA
  source 1..3189: mol_type = unassigned DNA
agtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 6 - protein, 664 aa, Homo sapiens
  REGION 1..664: note = protein-arginine deiminase type-3 [Homo sapiens]
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 7 - DNA, 3189 bp, Homo sapiens
  misc_feature 1..3189: note = Homo sapiens peptidyl arginine deiminase 3 (PADI3)transcript, mRNA
  source 1..3189: mol_type = unassigned DNA
agtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacgtctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 8 - protein, 664 aa, Homo sapiens
  REGION 1..664: note = protein-arginine deiminase type-3 [Homo sapiens]
MSLQRIVRVSLEHPTSAVCVAGVETLVDIYGSVPEGTEMFEVYGTPGVDIYVSPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 9 - DNA, 3189 bp, Homo sapiens
  misc_feature 1..3189: note = Homo sapiens peptidyl arginine deiminase 3 (PADI3)transcript, mRNA
  source 1..3189: mol_type = unassigned DNA
agtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctaccacacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 10 - protein, 664 aa, Homo sapiens
  REGION 1..664: note = protein-arginine deiminase type-3 [Homo sapiens]
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYHTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 11 - DNA, 3189 bp, Homo sapiens
  misc_feature 1..3189: note = Homo sapiens peptidyl arginine deiminase 3 (PADI3) transcript, mRNA
  source 1..3189: mol_type = unassigned DNA
agtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacatgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 12 - protein, 664 aa, Homo sapiens
  REGION 1..664: note = protein-arginine deiminase type-3 [Homo sapiens]
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHMHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 13 - DNA, 3189 bp, Homo sapiens
  misc_feature 1..3189: note = Homo sapiens peptidyl arginine deiminase 3 (PADI3)transcript, mRNA
  source 1..3189: mol_type = unassigned DNA
agtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggtaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 14 - protein, 664 aa, Homo sapiens
  REGION 1..664: note = protein-arginine deiminase type-3 [Homo sapiens]
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVVPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 15 - DNA, 3189 bp, Homo sapiens
  misc_feature 1..3189: note = Homo sapiens peptidyl arginine deiminase 3 (PADI3) transcript, mRNA
  source 1..3189: mol_type = unassigned DNA
agtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaaacaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 16 - protein, 664 aa, Homo sapiens
  REGION 1..664: note = protein-arginine deiminase type-3 [Homo sapiens]
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATTFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 17 - DNA, 3189 bp, Homo sapiens
  misc_feature 1..3189: note = Homo sapiens peptidyl arginine deiminase 3 (PADI3)transcript, mRNA
  source 1..3189: mol_type = unassigned DNA
agtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatacccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 18 - protein, 664 aa, Homo sapiens
  REGION 1..664: note = protein-arginine deiminase type-3 [Homo sapiens]
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGTIINGCCCLEEKVRSLLEPLGLHCTFIDDFTPYHMLHGEVHCGTNVCRKPFSFKWWNMVP

SEQ ID NO: 19 - DNA, 3189 bp, Homo sapiens
  misc_feature 1..3189: note = Homo sapiens peptidyl arginine deiminase 3 (PADI3)transcript, mRNA
  source 1..3189: mol_type = unassigned DNA
agtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcagtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 20 - protein, 664 aa, Homo sapiens
  REGION 1..664: note = protein-arginine deiminase type-3 [Homo sapiens]
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVQSLLEPLGLHCTFIDDFTPYHMLHGEVHCGTNVCRKPFSFKWWNMVP

SEQ ID NO: 21 - DNA, 4800 bp, Homo sapiens
  misc_feature 1..4800: note = PREDICTED: Homo sapiens peptidyl arginine deiminase 3 (PADI3), transcript variant X1, mRNA
  source 1..4800: mol_type = unassigned DNA
tctccctggttctgccatttcccctgtggggggctgcccagggccaaacaggccagagggctccaccagccctggaaattacaggctttgggagtagggttgccagataaaataaaagatgcctagttaaaattgaatttcaggtaaataattaataatttttttagtataagtgtatcccaaatacttcacgggagtaccaaacaatgattgactgttcacttgaaattcaagtccaactgagctttctgtatttttatttgctaagtctggcagccctgcttaggaggagaaacaggtgtgtctgtccctcccaactggtgaggacacctgacaggggcccagacaccagagtgggacctgcctgatggagagaagcttgggcttggtttatttcccttttcataaaaaagctgctttgagatgtaagagaatcatgttggtggaagagtgaagaggttgttttgggggaggtctgccttactctgagggaagatgggaagcggggggagtggccagctgtggaaatttggggtagtcccctacgtatcctgtgcctcagtttccccatgtgtaaacaggagtaattatctcatcctggggctgcagtgaggatcagatgagctaatgtacaaaaaggcttaggcaggcctgccgtgataagagtgcagaggctgggggctttttatgttatggtattgttgttattattactcctggggaggtggtgggcaagagtgtggactgaggagccacatggctgggtttggatcccagcccggccacagaccttgggcaagttacttaacctcggctttctcatctgtataatgggagacaataatagatagtgcctcccttctagggttattgagaggattgcatgatgggctggcacacagcatgagctcagtaagtgtcagtcactgttatcccccaggggcatgcatgccgctgcctccaggaggccctccaactcgcagtggcctctccctacatgggttcccactgagctgtgatcctgtggccttgagccccatctggctgccctggacaatgttggtcctcagctccccagcgggatcaacagtggggagagcagtgggtggatctgcccgccaggcagtacctgagtgcacagtgggcaccccacagtcaaaggatgcatagtctggtttccaaggaggaaccccaggctaggtggaatgagggaaatgcaatctcagggccagataacatcacagatactttaaacctgcaattaatttctggttgtttgaccaaatccggccaagtttcccgggtgacactgcagagctgtggaacagcatgggcttggagtcagacctgttgcacatctggctctgccatgtgatgcaacctcaggcttgtcacgaggcctccttgagcctcagttgctaatatgccaaatggggatgatattgctcaccttacagcttgtaagagtggagggagctgcgtgcgccaggtgcctggcatgcagcgggcactcaaccagattgaattcccttcccagggaagaattcaaggatatcttcccacccactaaggagacatgtgatgggatctggggtgagaattgctctttttaaagctctcagaactgtaaggggctccacccaccaatggctggtgttcactgagctcttcgtgcccagacctgcactggggatttatcgagcacatggctggcttctgacttccgaagagctcgccattctggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 22 - protein, 626 aa, Homo sapiens
  REGION 1..626: note = protein-arginine deiminase type-3 isoform X1 [Homo sapiens]
MFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 23 - DNA, 4800 bp, Homo sapiens
  misc_feature 1..4800: note = PREDICTED: Homo sapiens peptidyl arginine deiminase 3 (PADI3), transcript variant X1, mRNA
  source 1..4800: mol_type = unassigned DNA
tctccctggttctgccatttcccctgtggggggctgcccagggccaaacaggccagagggctccaccagccctggaaattacaggctttgggagtagggttgccagataaaataaaagatgcctagttaaaattgaatttcaggtaaataattaataatttttttagtataagtgtatcccaaatacttcacgggagtaccaaacaatgattgactgttcacttgaaattcaagtccaactgagctttctgtatttttatttgctaagtctggcagccctgcttaggaggagaaacaggtgtgtctgtccctcccaactggtgaggacacctgacaggggcccagacaccagagtgggacctgcctgatggagagaagcttgggcttggtttatttcccttttcataaaaaagctgctttgagatgtaagagaatcatgttggtggaagagtgaagaggttgttttgggggaggtctgccttactctgagggaagatgggaagcggggggagtggccagctgtggaaatttggggtagtcccctacgtatcctgtgcctcagtttccccatgtgtaaacaggagtaattatctcatcctggggctgcagtgaggatcagatgagctaatgtacaaaaaggcttaggcaggcctgccgtgataagagtgcagaggctgggggctttttatgttatggtattgttgttattattactcctggggaggtggtgggcaagagtgtggactgaggagccacatggctgggtttggatcccagcccggccacagaccttgggcaagttacttaacctcggctttctcatctgtataatgggagacaataatagatagtgcctcccttctagggttattgagaggattgcatgatgggctggcacacagcatgagctcagtaagtgtcagtcactgttatcccccaggggcatgcatgccgctgcctccaggaggccctccaactcgcagtggcctctccctacatgggttcccactgagctgtgatcctgtggccttgagccccatctggctgccctggacaatgttggtcctcagctccccagcgggatcaacagtggggagagcagtgggtggatctgcccgccaggcagtacctgagtgcacagtgggcaccccacagtcaaaggatgcatagtctggtttccaaggaggaaccccaggctaggtggaatgagggaaatgcaatctcagggccagataacatcacagatactttaaacctgcaattaatttctggttgtttgaccaaatccggccaagtttcccgggtgacactgcagagctgtggaacagcatgggcttggagtcagacctgttgcacatctggctctgccatgtgatgcaacctcaggcttgtcacgaggcctccttgagcctcagttgctaatatgccaaatggggatgatattgctcaccttacagcttgtaagagtggagggagctgcgtgcgccaggtgcctggcatgcagcgggcactcaaccagattgaattcccttcccagggaagaattcaaggatatcttcccacccactaaggagacatgtgatgggatctggggtgagaattgctctttttaaagctctcagaactgtaaggggctccacccaccaatggctggtgttcactgagctcttcgtgcccagacctgcactggggatttatcgagcacatggctggcttctgacttccgaagagctcgccattctggtcagtgcctgagggcacagaaatgtttgaggtctatgggacgcctggcgtggacatctacgtctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 24 - protein, 626 aa, Homo sapiens [sequence omitted]

SEQ ID NO: 25 - DNA, 4800 bp, Homo sapiens
  misc_feature 1..4800: note = PREDICTED: Homo sapiens peptidyl arginine deiminase 3 (PADI3), transcript variant X1, mRNA
  source 1..4800: mol_type = unassigned DNA
tctccctggttctgccatttcccctgtggggggctgcccagggccaaacaggccagagggctccaccagccctggaaattacaggctttgggagtagggttgccagataaaataaaagatgcctagttaaaattgaatttcaggtaaataattaataatttttttagtataagtgtatcccaaatacttcacgggagtaccaaacaatgattgactgttcacttgaaattcaagtccaactgagctttctgtatttttatttgctaagtctggcagccctgcttaggaggagaaacaggtgtgtctgtccctcccaactggtgaggacacctgacaggggcccagacaccagagtgggacctgcctgatggagagaagcttgggcttggtttatttcccttttcataaaaaagctgctttgagatgtaagagaatcatgttggtggaagagtgaagaggttgttttgggggaggtctgccttactctgagggaagatgggaagcggggggagtggccagctgtggaaatttggggtagtcccctacgtatcctgtgcctcagtttccccatgtgtaaacaggagtaattatctcatcctggggctgcagtgaggatcagatgagctaatgtacaaaaaggcttaggcaggcctgccgtgataagagtgcagaggctgggggctttttatgttatggtattgttgttattattactcctggggaggtggtgggcaagagtgtggactgaggagccacatggctgggtttggatcccagcccggccacagaccttgggcaagttacttaacctcggctttctcatctgtataatgggagacaataatagatagtgcctcccttctagggttattgagaggattgcatgatgggctggcacacagcatgagctcagtaagtgtcagtcactgttatcccccaggggcatgcatgccgctgcctccaggaggccctccaactcgcagtggcctctccctacatgggttcccactgagctgtgatcctgtggccttgagccccatctggctgccctggacaatgttggtcctcagctccccagcgggatcaacagtggggagagcagtgggtggatctgcccgccaggcagtacctgagtgcacagtgggcaccccacagtcaaaggatgcatagtctggtttccaaggaggaaccccaggctaggtggaatgagggaaatgcaatctcagggccagataacatcacagatactttaaacctgcaattaatttctggttgtttgaccaaatccggccaagtttcccgggtgacactgcagagctgtggaacagcatgggcttggagtcagacctgttgcacatctggctctgccatgtgatgcaacctcaggcttgtcacgaggcctccttgagcctcagttgctaatatgccaaatggggatgatattgctcaccttacagcttgtaagagtggagggagctgcgtgcgccaggtgcctggcatgcagcgggcactcaaccagattgaattcccttcccagggaagaattcaaggatatcttcccacccactaaggagacatgtgatgggatctggggtgagaattgctctttttaaagctctcagaactgtaaggggctccacccaccaatggctggtgttcactgagctcttcgtgcccagacctgcactggggatttatcgagcacatggctggcttctgacttccgaagagctcgccattctggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctaccacacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 26 - protein, 626 aa, Homo sapiens
  REGION 1..626: note = protein-arginine deiminase type-3 isoform X1 [Homo sapiens]
MFEVYGTPGVDIYISPNMERGRERADTRRWRFDATLEIIVVMNSPSNDLNDSHVQISYHSSHEPLPLAYAVLYHTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 27 - DNA, 4800 bp, Homo sapiens
  misc_feature 1..4800: note = PREDICTED: Homo sapiens peptidyl arginine deiminase 3 (PADI3), transcript variant X1, mRNA
  source 1..4800: mol_type = unassigned DNA
tctccctggttctgccatttcccctgtggggggctgcccagggccaaacaggccagagggctccaccagccctggaaattacaggctttgggagtagggttgccagataaaataaaagatgcctagttaaaattgaatttcaggtaaataattaataatttttttagtataagtgtatcccaaatacttcacgggagtaccaaacaatgattgactgttcacttgaaattcaagtccaactgagctttctgtatttttatttgctaagtctggcagccctgcttaggaggagaaacaggtgtgtctgtccctcccaactggtgaggacacctgacaggggcccagacaccagagtgggacctgcctgatggagagaagcttgggcttggtttatttcccttttcataaaaaagctgctttgagatgtaagagaatcatgttggtggaagagtgaagaggttgttttgggggaggtctgccttactctgagggaagatgggaagcggggggagtggccagctgtggaaatttggggtagtcccctacgtatcctgtgcctcagtttccccatgtgtaaacaggagtaattatctcatcctggggctgcagtgaggatcagatgagctaatgtacaaaaaggcttaggcaggcctgccgtgataagagtgcagaggctgggggctttttatgttatggtattgttgttattattactcctggggaggtggtgggcaagagtgtggactgaggagccacatggctgggtttggatcccagcccggccacagaccttgggcaagttacttaacctcggctttctcatctgtataatgggagacaataatagatagtgcctcccttctagggttattgagaggattgcatgatgggctggcacacagcatgagctcagtaagtgtcagtcactgttatcccccaggggcatgcatgccgctgcctccaggaggccctccaactcgcagtggcctctccctacatgggttcccactgagctgtgatcctgtggccttgagccccatctggctgccctggacaatgttggtcctcagctccccagcgggatcaacagtggggagagcagtgggtggatctgcccgccaggcagtacctgagtgcacagtgggcaccccacagtcaaaggatgcatagtctggtttccaaggaggaaccccaggctaggtggaatgagggaaatgcaatctcagggccagataacatcacagatactttaaacctgcaattaatttctggttgtttgaccaaatccggccaagtttcccgggtgacactgcagagctgtggaacagcatgggcttggagtcagacctgttgcacatctggctctgccatgtgatgcaacctcaggcttgtcacgaggcctccttgagcctcagttgctaatatgccaaatggggatgatattgctcaccttacagcttgtaagagtggagggagctgcgtgcgccaggtgcctggcatgcagcgggcactcaaccagattgaattcccttcccagggaagaattcaaggatatcttcccacccactaaggagacatgtgatgggatctggggtgagaattgctctttttaaagctctcagaactgtaaggggctccacccaccaatggctggtgttcactgagctcttcgtgcccagacctgcactggggatttatcgagcacatggctggcttctgacttccgaagagctcgccattctggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacatgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 28 - protein, 626 aa, Homo sapiens
  REGION 1..626: note = protein-arginine deiminase type-3 isoform X1 [Homo sapiens]
MFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHMHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 29 - DNA, 4800 bp, Homo sapiens
  misc_feature 1..4800: note = PREDICTED: Homo sapiens peptidyl arginine deiminase 3 (PADI3), transcript variant X1, mRNA
  source 1..4800: mol_type = unassigned DNA
tctccctggttctgccatttcccctgtggggggctgcccagggccaaacaggccagagggctccaccagccctggaaattacaggctttgggagtagggttgccagataaaataaaagatgcctagttaaaattgaatttcaggtaaataattaataatttttttagtataagtgtatcccaaatacttcacgggagtaccaaacaatgattgactgttcacttgaaattcaagtccaactgagctttctgtatttttatttgctaagtctggcagccctgcttaggaggagaaacaggtgtgtctgtccctcccaactggtgaggacacctgacaggggcccagacaccagagtgggacctgcctgatggagagaagcttgggcttggtttatttcccttttcataaaaaagctgctttgagatgtaagagaatcatgttggtggaagagtgaagaggttgttttgggggaggtctgccttactctgagggaagatgggaagcggggggagtggccagctgtggaaatttggggtagtcccctacgtatcctgtgcctcagtttccccatgtgtaaacaggagtaattatctcatcctggggctgcagtgaggatcagatgagctaatgtacaaaaaggcttaggcaggcctgccgtgataagagtgcagaggctgggggctttttatgttatggtattgttgttattattactcctggggaggtggtgggcaagagtgtggactgaggagccacatggctgggtttggatcccagcccggccacagaccttgggcaagttacttaacctcggctttctcatctgtataatgggagacaataatagatagtgcctcccttctagggttattgagaggattgcatgatgggctggcacacagcatgagctcagtaagtgtcagtcactgttatcccccaggggcatgcatgccgctgcctccaggaggccctccaactcgcagtggcctctccctacatgggttcccactgagctgtgatcctgtggccttgagccccatctggctgccctggacaatgttggtcctcagctccccagcgggatcaacagtggggagagcagtgggtggatctgcccgccaggcagtacctgagtgcacagtgggcaccccacagtcaaaggatgcatagtctggtttccaaggaggaaccccaggctaggtggaatgagggaaatgcaatctcagggccagataacatcacagatactttaaacctgcaattaatttctggttgtttgaccaaatccggccaagtttcccgggtgacactgcagagctgtggaacagcatgggcttggagtcagacctgttgcacatctggctctgccatgtgatgcaacctcaggcttgtcacgaggcctccttgagcctcagttgctaatatgccaaatggggatgatattgctcaccttacagcttgtaagagtggagggagctgcgtgcgccaggtgcctggcatgcagcgggcactcaaccagattgaattcccttcccagggaagaattcaaggatatcttcccacccactaaggagacatgtgatgggatctggggtgagaattgctctttttaaagctctcagaactgtaaggggctccacccaccaatggctggtgttcactgagctcttcgtgcccagacctgcactggggatttatcgagcacatggctggcttctgacttccgaagagctcgccattctggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggtaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 30 - protein, 626 aa, Homo sapiens
  REGION 1..626: note = protein-arginine deiminase type-3 isoform X1 [Homo sapiens]
MFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVVPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 31 - DNA, 4800 bp, Homo sapiens
  misc_feature 1..4800: note = PREDICTED: Homo sapiens peptidyl arginine deiminase 3 (PADI3), transcript variant X1, mRNA
  source 1..4800: mol_type = unassigned DNA
tctccctggttctgccatttcccctgtggggggctgcccagggccaaacaggccagagggctccaccagccctggaaattacaggctttgggagtagggttgccagataaaataaaagatgcctagttaaaattgaatttcaggtaaataattaataatttttttagtataagtgtatcccaaatacttcacgggagtaccaaacaatgattgactgttcacttgaaattcaagtccaactgagctttctgtatttttatttgctaagtctggcagccctgcttaggaggagaaacaggtgtgtctgtccctcccaactggtgaggacacctgacaggggcccagacaccagagtgggacctgcctgatggagagaagcttgggcttggtttatttcccttttcataaaaaagctgctttgagatgtaagagaatcatgttggtggaagagtgaagaggttgttttgggggaggtctgccttactctgagggaagatgggaagcggggggagtggccagctgtggaaatttggggtagtcccctacgtatcctgtgcctcagtttccccatgtgtaaacaggagtaattatctcatcctggggctgcagtgaggatcagatgagctaatgtacaaaaaggcttaggcaggcctgccgtgataagagtgcagaggctgggggctttttatgttatggtattgttgttattattactcctggggaggtggtgggcaagagtgtggactgaggagccacatggctgggtttggatcccagcccggccacagaccttgggcaagttacttaacctcggctttctcatctgtataatgggagacaataatagatagtgcctcccttctagggttattgagaggattgcatgatgggctggcacacagcatgagctcagtaagtgtcagtcactgttatcccccaggggcatgcatgccgctgcctccaggaggccctccaactcgcagtggcctctccctacatgggttcccactgagctgtgatcctgtggccttgagccccatctggctgccctggacaatgttggtcctcagctccccagcgggatcaacagtggggagagcagtgggtggatctgcccgccaggcagtacctgagtgcacagtgggcaccccacagtcaaaggatgcatagtctggtttccaaggaggaaccccaggctaggtggaatgagggaaatgcaatctcagggccagataacatcacagatactttaaacctgcaattaatttctggttgtttgaccaaatccggccaagtttcccgggtgacactgcagagctgtggaacagcatgggcttggagtcagacctgttgcacatctggctctgccatgtgatgcaacctcaggcttgtcacgaggcctccttgagcctcagttgctaatatgccaaatggggatgatattgctcaccttacagcttgtaagagtggagggagctgcgtgcgccaggtgcctggcatgcagcgggcactcaaccagattgaattcccttcccagggaagaattcaaggatatcttcccacccactaaggagacatgtgatgggatctggggtgagaattgctctttttaaagctctcagaactgtaaggggctccacccaccaatggctggtgttcactgagctcttcgtgcccagacctgcactggggatttatcgagcacatggctggcttctgacttccgaagagctcgccattctggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacgaccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 32 - protein, 626 aa, Homo sapiens
  REGION 1..626: note = protein-arginine deiminase type-3 isoform X1 [Homo sapiens]
MFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATTFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 33 - DNA, 4800 bp, Homo sapiens
  misc_feature 1..4800: note = PREDICTED: Homo sapiens peptidyl arginine deiminase 3 (PADI3), transcript variant X1, mRNA
  source 1..4800: mol_type = unassigned DNA
tctccctggttctgccatttcccctgtggggggctgcccagggccaaacaggccagagggctccaccagccctggaaattacaggctttgggagtagggttgccagataaaataaaagatgcctagttaaaattgaatttcaggtaaataattaataatttttttagtataagtgtatcccaaatacttcacgggagtaccaaacaatgattgactgttcacttgaaattcaagtccaactgagctttctgtatttttatttgctaagtctggcagccctgcttaggaggagaaacaggtgtgtctgtccctcccaactggtgaggacacctgacaggggcccagacaccagagtgggacctgcctgatggagagaagcttgggcttggtttatttcccttttcataaaaaagctgctttgagatgtaagagaatcatgttggtggaagagtgaagaggttgttttgggggaggtctgccttactctgagggaagatgggaagcggggggagtggccagctgtggaaatttggggtagtcccctacgtatcctgtgcctcagtttccccatgtgtaaacaggagtaattatctcatcctggggctgcagtgaggatcagatgagctaatgtacaaaaaggcttaggcaggcctgccgtgataagagtgcagaggctgggggctttttatgttatggtattgttgttattattactcctggggaggtggtgggcaagagtgtggactgaggagccacatggctgggtttggatcccagcccggccacagaccttgggcaagttacttaacctcggctttctcatctgtataatgggagacaataatagatagtgcctcccttctagggttattgagaggattgcatgatgggctggcacacagcatgagctcagtaagtgtcagtcactgttatcccccaggggcatgcatgccgctgcctccaggaggccctccaactcgcagtggcctctccctacatgggttcccactgagctgtgatcctgtggccttgagccccatctggctgccctggacaatgttggtcctcagctccccagcgggatcaacagtggggagagcagtgggtggatctgcccgccaggcagtacctgagtgcacagtgggcaccccacagtcaaaggatgcatagtctggtttccaaggaggaaccccaggctaggtggaatgagggaaatgcaatctcagggccagataacatcacagatactttaaacctgcaattaatttctggttgtttgaccaaatccggccaagtttcccgggtgacactgcagagctgtggaacagcatgggcttggagtcagacctgttgcacatctggctctgccatgtgatgcaacctcaggcttgtcacgaggcctccttgagcctcagttgctaatatgccaaatggggatgatattgctcaccttacagcttgtaagagtggagggagctgcgtgcgccaggtgcctggcatgcagcgggcactcaaccagattgaattcccttcccagggaagaattcaaggatatcttcccacccactaaggagacatgtgatgggatctggggtgagaattgctctttttaaagctctcagaactgtaaggggctccacccaccaatggctggtgttcactgagctcttcgtgcccagacctgcactggggatttatcgagcacatggctggcttctgacttccgaagagctcgccattctggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggaccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 34 - protein, 626 aa, Homo sapiens
  REGION 1..626: note = protein-arginine deiminase type-3 isoform X1 [Homo sapiens]
MFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGTIINGCCCLEEKVRSLLEPLGLHCTFIDDFTPYHMLHGEVHCGTNVCRKPFSFKWWNMVP

SEQ ID NO: 35 - DNA, 4800 bp, Homo sapiens
  misc_feature 1..4800: note = PREDICTED: Homo sapiens peptidyl arginine deiminase 3 (PADI3), transcript variant X1, mRNA
  source 1..4800: mol_type = unassigned DNA
tctccctggttctgccatttcccctgtggggggctgcccagggccaaacaggccagagggctccaccagccctggaaattacaggctttgggagtagggttgccagataaaataaaagatgcctagttaaaattgaatttcaggtaaataattaataatttttttagtataagtgtatcccaaatacttcacgggagtaccaaacaatgattgactgttcacttgaaattcaagtccaactgagctttctgtatttttatttgctaagtctggcagccctgcttaggaggagaaacaggtgtgtctgtccctcccaactggtgaggacacctgacaggggcccagacaccagagtgggacctgcctgatggagagaagcttgggcttggtttatttcccttttcataaaaaagctgctttgagatgtaagagaatcatgttggtggaagagtgaagaggttgttttgggggaggtctgccttactctgagggaagatgggaagcggggggagtggccagctgtggaaatttggggtagtcccctacgtatcctgtgcctcagtttccccatgtgtaaacaggagtaattatctcatcctggggctgcagtgaggatcagatgagctaatgtacaaaaaggcttaggcaggcctgccgtgataagagtgcagaggctgggggctttttatgttatggtattgttgttattattactcctggggaggtggtgggcaagagtgtggactgaggagccacatggctgggtttggatcccagcccggccacagaccttgggcaagttacttaacctcggctttctcatctgtataatgggagacaataatagatagtgcctcccttctagggttattgagaggattgcatgatgggctggcacacagcatgagctcagtaagtgtcagtcactgttatcccccaggggcatgcatgccgctgcctccaggaggccctccaactcgcagtggcctctccctacatgggttcccactgagctgtgatcctgtggccttgagccccatctggctgccctggacaatgttggtcctcagctccccagcgggatcaacagtggggagagcagtgggtggatctgcccgccaggcagtacctgagtgcacagtgggcaccccacagtcaaaggatgcatagtctggtttccaaggaggaaccccaggctaggtggaatgagggaaatgcaatctcagggccagataacatcacagatactttaaacctgcaattaatttctggttgtttgaccaaatccggccaagtttcccgggtgacactgcagagctgtggaacagcatgggcttggagtcagacctgttgcacatctggctctgccatgtgatgcaacctcaggcttgtcacgaggcctccttgagcctcagttgctaatatgccaaatggggatgatattgctcaccttacagcttgtaagagtggagggagctgcgtgcgccaggtgcctggcatgcagcgggcactcaaccagattgaattcccttcccagggaagaattcaaggatatcttcccacccactaaggagacatgtgatgggatctggggtgagaattgctctttttaaagctctcagaactgtaaggggctccacccaccaatggctggtgttcactgagctcttcgtgcccagacctgcactggggatttatcgagcacatggctggcttctgacttccgaagagctcgccattctggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcagtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 36 - protein, 626 aa, Homo sapiens
  REGION 1..626: note = protein-arginine deiminase type-3 isoform X1 [Homo sapiens]
MFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVQSLLEPLGLHCTFIDDFTPYHMLHGEVHCGTNVCRKPFSFKWWNMVP

SEQ ID NO: 37 - DNA, 2938 bp, Homo sapiens
  misc_feature 1..2938: note = PREDICTED: Homo sapiens peptidyl arginine deiminase 3 (PADI3), transcript variant X2, mRNA
  source 1..2938: mol_type = unassigned DNA
agaaatggatggatgtgactgtgtgctaataaaactttatttatacaaacaggcagtaggccagatttggcccacagttcataatgtgctgatcctgacctaggcgagaagagaaaccaaatatgaaactgttgaagaacttgggactgaattatgttggaacttggtgccctgggagttaagaggagaaggtcgagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 38 - protein, 485 aa, Homo sapiens
  REGION 1..485: note = protein-arginine deiminase type-3 isoform X2 [Homo sapiens]
MSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 39 - DNA, 2938 bp, Homo sapiens; identical to SEQ ID NO: 37
  misc_feature 1..2938: note = PREDICTED: Homo sapiens peptidyl arginine deiminase 3 (PADI3), transcript variant X2, mRNA
  source 1..2938: mol_type = unassigned DNA

SEQ ID NO: 40 - DNA, 2938 bp, Homo sapiens; identical to SEQ ID NO: 37
  misc_feature 1..2938: note = PREDICTED: Homo sapiens peptidyl arginine deiminase 3 (PADI3), transcript variant X2, mRNA
  source 1..2938: mol_type = unassigned DNA

SEQ ID NO: 41 - DNA, 2938 bp, Homo sapiens
  misc_feature 1..2938: note = PREDICTED: Homo sapiens peptidyl arginine deiminase 3 (PADI3), transcript variant X2, mRNA
  source 1..2938: mol_type = unassigned DNA
agaaatggatggatgtgactgtgtgctaataaaactttatttatacaaacaggcagtaggccagatttggcccacagttcataatgtgctgatcctgacctaggcgagaagagaaaccaaatatgaaactgttgaagaacttgggactgaattatgttggaacttggtgccctgggagttaagaggagaaggtcgagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggtaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 42 - protein, 485 aa, Homo sapiens
  REGION 1..485: note = protein-arginine deiminase type-3 isoform X2 [Homo sapiens]
MSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVVPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 43 - DNA, 2938 bp, Homo sapiens
  misc_feature 1..2938: note = PREDICTED: Homo sapiens peptidyl arginine deiminase 3 (PADI3), transcript variant X2, mRNA
  source 1..2938: mol_type = unassigned DNA
agaaatggatggatgtgactgtgtgctaataaaactttatttatacaaacaggcagtaggccagatttggcccacagttcataatgtgctgatcctgacctaggcgagaagagaaaccaaatatgaaactgttgaagaacttgggactgaattatgttggaacttggtgccctgggagttaagaggagaaggtcgagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacgaccttcttccctgacttggtgaacatgctggtgctggggaagcacctgggcatccccaagccctttgggcccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 44 - protein, 485 aa, Homo sapiens
  REGION 1..485: note = protein-arginine deiminase type-3 isoform X2 [Homo sapiens]
MSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATTFFPDLVNMLVLGKHLGIPKPFGPIINGCCCLEEKVRSLLEPLGLHCTFIDDFTPYHMLHGEVHCGTNVCRKPFSFKWWNMVP

SEQ ID NO: 45 - DNA, 2938 bp, Homo sapiens
  misc_feature 1..2938: note = PREDICTED: Homo sapiens peptidyl arginine deiminase 3 (PADI3), transcript variant X2, mRNA
  source 1..2938: mol_type = unassigned DNA
agaaatggatggatgtgactgtgtgctaataaaactttatttatacaaacaggcagtaggccagatttggcccacagttcataatgtgctgatcctgacctaggcgagaagagaaaccaaatatgaaactgttgaagaacttgggactgaattatgttggaacttggtgccctgggagttaagaggagaaggtcgagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggaccatcatcaatggctgctgctgcctggaggagaaggtgcggtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 46 - protein, 485 aa, Homo sapiens
  REGION 1..485: note = protein-arginine deiminase type-3 isoform X2 [Homo sapiens]
MSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGTIINGCCCLEEKVRSLLEPLGLHCTFIDDFTPYHMLHGEVHCGTNVCRKPFSFKWWNMVP

SEQ ID NO: 47 - DNA, 2938 bp, Homo sapiens
  misc_feature 1..2938: note = PREDICTED: Homo sapiens peptidyl arginine deiminase 3 (PADI3), transcript variant X2, mRNA
  source 1..2938: mol_type = unassigned DNA
agaaatggatggatgtgactgtgtgctaataaaactttatttatacaaacaggcagtaggccagatttggcccacagttcataatgtgctgatcctgacctaggcgagaagagaaaccaaatatgaaactgttgaagaacttgggactgaattatgttggaacttggtgccctgggagttaagaggagaaggtcgagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctgggtccagattttggttacgtgactcgggaaccacgcgacaggtctgtgagtggcctggactcctttgggaacctggaggtcagccctccagtggtggccaatgggaaagagtaccccctggggaggatcctcattgggggcaacctgcctgggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccagggggttgttgatgatgagcaggtcaagaccatctccatcaaccaggtgctctccaataaagacctcatcaactacaataagtttgtgcagagctgcatcgactggaaccgtgaggtgctgaagcgggagctgggcctggcagagtgtgacatcattgacatcccacagctcttcaagaccgagaggaaaaaagcaacggccttcttccctgacttggtgaacatgctggtgctggggaagcacctgggcatccccaagccctttgggcccatcatcaatggctgctgctgcctggaggagaaggtgcagtccctgctggagccgctgggcctccactgcaccttcattgatgacttcactccataccacatgctgcatggggaggtgcactgtggcaccaatgtgtgcagaaagcccttctctttcaagtggtggaacatggtgccctgagacagctcccacccaccatcctgtccccctggggcgggcattggcccaggtggtggagacagagacaggcccctgaacgataagcaccaagagaccccaaggctccagatggaacactgagggtgaccgtccctctcagaagccttttccctggaagtgtccatgcctcacctgcaacccatgtggttctcagacttgaatcttctcggccccccaaaaagaaggacctcatttcttatagcctctcctgtgattcaacacaacccatggagatgtccccttctcactctgaaatcatccatttggggacaaatccacattggggtctagaaacatccacgtatctcatcagccatcttgtcctgtgcatcctaacagaggaaggatccatgattctgctttggtccaattgcttcctctctgcagaggaacaaccctaaaaccagaccactccacgcaggacaggcaggagagattcttcctaaagcctcccccataaaaagggagctgtggatccacttagatcagggcggaaccatctttcacccggccaagctcctgcccagatgttgaccctcacccagcgtgagctgtcacatagtaggagcttctagatgcatgtggaagcaatgagagttgtcccttagccttataaactccccatgatctgacatgcagaaatccagccttgtccagaatcctcctggaatttcttggagacgaaagtatctgggggattgttgggtactagggagactgggtacaagggtgaaaagtagttcccataatacacatggttgactatggtgatccaccttgtgatggttaatattaggtgtctggagaaggttgcttcattggccctgggacttctctctgcaggaggagagaacgctgcctctcctctggattggtctcaggctctctgttggcctttggtcagcgtttccacatcctgctctgctgcaggagagggggctaaggggctggatccaccaaggcagctcacagcgggaaaactctgggaatgaaccactgaattcaggggatgggggtgggggggcggttctcgaggtgtgtgccagctacacgtgtgttctgtatgggtccagctgcgtttccatcactcgctaataaatcaacagaaacacaaa

SEQ ID NO: 48 - protein, 485 aa, Homo sapiens
  REGION 1..485: note = protein-arginine deiminase type-3 isoform X2 [Homo sapiens]
MSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILGPDFGYVTREPRDRSVSGLDSFGNLEVSPPVVANGKEYPLGRILIGGNLPGSSGRRVTQVVRDFLHAQKVQPPVELFVDWLAVGHVDEFLSFVPAPDGKGFRMLLASPGACFKLFQEKQKCGHGRALLFQGVVDDEQVKTISINQVLSNKDLINYNKFVQSCIDWNREVLKRELGLAECDIIDIPQLFKTERKKATAFFPDLVNMLVLGKHLGIPKPFGPIINGCCCLEEKVQSLLEPLGLHCTFIDDFTPYHMLHGEVHCGTNVCRKPFSFKWWNMVP

SEQ ID NO: 49 - DNA, 1452 bp, Homo sapiens
  misc_feature 1..1452: note = PREDICTED: Homo sapiens peptidyl arginine deiminase 3 (PADI3), transcript variant X3, mRNA
  source 1..1452: mol_type = unassigned DNA
cctaaggggcctcaggggcagtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccag

SEQ ID NO: 50 - protein, 421 aa, Homo sapiens
  REGION 1..421: note = protein-arginine deiminase type-3 isoform X3 [Homo sapiens]
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILVKWPQGHPGGAGLPPCPEGAAPRGALCGLVGRGPCG

SEQ ID NO: 51 - DNA, 1452 bp, Homo sapiens
  misc_feature 1..1452: note = PREDICTED: Homo sapiens peptidyl arginine deiminase 3 (PADI3), transcript variant X3, mRNA
  source 1..1452: mol_type = unassigned DNA
cctaaggggcctcaggggcagtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacgtctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccag

SEQ ID NO: 52 - protein, 421 aa, Homo sapiens
  REGION 1..421: note = protein-arginine deiminase type-3 isoform X3 [Homo sapiens]
MSLQRIVRVSLEHPTSAVCVAGVETLVDIYGSVPEGTEMFEVYGTPGVDIYVSPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILVKWPQGHPGGAGLPPCPEGAAPRGALCGLVGRGPCG

SEQ ID NO: 53 - DNA, 1452 bp, Homo sapiens
  misc_feature 1..1452: note = PREDICTED: Homo sapiens peptidyl arginine deiminase 3 (PADI3), transcript variant X3, mRNA
  source 1..1452: mol_type = unassigned DNA
cctaaggggcctcaggggcagtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctaccacacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccag

SEQ ID NO: 54 - protein, 421 aa, Homo sapiens
  REGION 1..421: note = protein-arginine deiminase type-3 isoform X3 [Homo sapiens]
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYHTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILVKWPQGHPGGAGLPPCPEGAAPRGALCGLVGRGPCG

SEQ ID NO: 55 - DNA, 1452 bp, Homo sapiens
  misc_feature 1..1452: note = PREDICTED: Homo sapiens peptidyl arginine deiminase 3 (PADI3), transcript variant X3, mRNA
  source 1..1452: mol_type = unassigned DNA
cctaaggggcctcaggggcagtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacatgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggcaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccag

SEQ ID NO: 56 - protein, 421 aa, Homo sapiens
  REGION 1..421: note = protein-arginine deiminase type-3 isoform X3 [Homo sapiens]
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHMHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVAPWIMTPSTLPPLEVYVCRVRNNTCFVDAVAELARKAGCKLTICPQAENRNDRWIQDEMELGYVQAPHKTLPVVFDSPRNGELQDFPYKRILVKWPQGHPGGAGLPPCPEGAAPRGALCGLVGRGPCG

SEQ ID NO: 57 - DNA, 1452 bp, Homo sapiens
  misc_feature 1..1452: note = PREDICTED: Homo sapiens peptidyl arginine deiminase 3 (PADI3), transcript variant X3, mRNA
  source 1..1452: mol_type = unassigned DNA
cctaaggggcctcaggggcagtgttggggttggcggccacagctaagtccaacaccagcatgtcgctgcagagaatcgtgcgtgtgtccctggagcatcccaccagcgcggtgtgtgtggctggcgtggagaccctcgtggacatttatgggtcagtgcctgagggcacagaaatgtttgaggtctatgggacgcctggcgtggacatctacatctctcccaacatggagaggggccgggagcgtgcagacaccaggcggtggcgctttgacgcgactttggagatcatcgtggtcatgaactcccccagcaatgacctcaacgacagccatgttcagatttcctaccactccagccatgagcctctgcccctggcctatgcggtgctctacctcacctgtgttgacatctctctggattgcgacctgaactgtgagggaaggcaggacaggaactttgtagacaagcggcagtgggtctgggggcccagtgggtatggcggcatcttgctggtgaactgtgaccgtgatgatccgagctgtgatgtccaggacaattgtgaccagcacgtgcactgcctgcaagacctggaagacatgtctgtcatggtcctgcggacgcagggccctgcagccctctttgatgaccacaaacttgtcctccatacctccagctatgatgccaaacgggcacaggtcttccacatctgcggtcctgaggatgtgtgtgaggcctataggcatgtgctgggccaagataaggtgtcctatgaggtaccccgcttgcatggggatgaggagcgcttcttcgtggaaggcctgtccttccctgatgccggcttcacaggactcatctccttccatgtcactctgctggacgactccaacgaggatttctcggcatcccctatcttcactgacactgtggtgttccgagtggtaccctggatcatgacgcccagcactctgccacccctagaggtgtatgtgtgccgtgtgaggaacaacacgtgttttgtggatgcggtggcagagctggccaggaaggccggctgcaagctgaccatctgcccacaggccgagaaccgcaacgaccgctggatccaggatgagatggagctgggctacgttcaggcgccgcacaagaccctcccggtggtctttgactccccaaggaatggggaactgcaggatttcccttacaaaagaatcctggtcaagtggccgcagggtcacccaggtggtgcgggacttcctccatgcccagaaggtgcagccccccgtggagctctttgtggactggttggccgtgggccatgtggatgagtttctgagctttgtccctgcccccgatgggaagggcttccggatgctcctggccagccctggggcctgcttcaagctcttccaggaaaagcagaagtgtggccacgggagggccctcctgttccag

SEQ ID NO: 58 - protein, 421 aa, Homo sapiens
  REGION 1..421: note = protein-arginine deiminase type-3 isoform X3 [Homo sapiens]
MSLQRIVRVSLEHPTSAVCVAGVETLVDIYGSVPEGTEMFEVYGTPGVDIYISPNMERGRERADTRRWRFDATLEIIVVMNSPSNDLNDSHVQISYHSSHEPLPLAYAVLYLTCVDISLDCDLNCEGRQDRNFVDKRQWVWGPSGYGGILLVNCDRDDPSCDVQDNCDQHVHCLQDLEDMSVMVLRTQGPAALFDDHKLVLHTSSYDAKRAQVFHICGPEDVCEAYRHVLGQDKVSYEVPRLHGDEERFFVEGLSFPDAGFTGLISFHVTLLDDSNEDFSASPIFTDTVVFRVVPWIMTPSTLPPLEVYVCRVRNNTCFVDAVAELARKAGCKLTICPQAENRNDRWIQDEMELGYVQAPHKTLPVVFDSPRNGELQDFPYKRILVKWPQGHPGGAGLPPCPEGAAPRGALCGLVGRGPCG

SEQ ID NO: 59 - DNA, 1452 bp, Homo sapiens; identical to SEQ ID NO: 49
  misc_feature 1..1452: note = PREDICTED: Homo sapiens peptidyl arginine deiminase 3 (PADI3), transcript variant X3, mRNA
  source 1..1452: mol_type = unassigned DNA

SEQ ID NO: 60 - skipped

SEQ ID NO: 61 - DNA, 2267 bp, Homo sapiens
  misc_feature 1..2267: note = Homo sapiens peptidyl arginine deiminase 4 (PADI4), mRNA
  source 1..2267: mol_type = unassigned DNA
agccagagggacgagctagcccgacgatggcccaggggacattgatccgtgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtacatga

SEQ ID NO: 62 - protein, 663 aa, Homo sapiens
  REGION 1..663: note = protein-arginine deiminase type-4 [Homo sapiens]
MAQGTLIRVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 63 - DNA, 2267 bp, Homo sapiens
  misc_feature 1..2267: note = Homo sapiens peptidyl arginine deiminase 4 (PADI4), mRNA
  source 1..2267: mol_type = unassigned DNA
agccagagggacgagctagcccgacgatggcccaggggacattgatccatgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtacatga

SEQ ID NO: 64 - protein, 663 aa, Homo sapiens
  REGION 1..663: note = protein-arginine deiminase type-4 [Homo sapiens]
MAQGTLIHVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 65 - DNA, 2267 bp, Homo sapiens
  misc_feature 1..2267: note = Homo sapiens peptidyl arginine deiminase 4 (PADI4), mRNA
  source 1..2267: mol_type = unassigned DNA
agccagagggacgagctagcccgacgatggcccaggggacattgatccttgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtacatga

SEQ ID NO: 66 - protein, 663 aa, Homo sapiens
  REGION 1..663: note = protein-arginine deiminase type-4 [Homo sapiens]
MAQGTLILVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 67 - DNA, 1801 bp, Homo sapiens
  misc_feature 1..1801: note = PREDICTED: Homo sapiens peptidyl arginine deiminase 4 (PADI4), transcript variant X3, mRNA
  source 1..1801: mol_type = unassigned DNA
tctacctcaccggggtggaaatctccttgtgcgcagacatcacccgcaccggcaaagtgaagccaaccagagctgtgaaagatcagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtacatga

SEQ ID NO: 68 - DNA, 1748 bp, Homo sapiens
  misc_feature 1..1748: note = PREDICTED: Homo sapiens peptidyl arginine deiminase 4 (PADI4), transcript variant X8, mRNA
  source 1..1748: mol_type = unassigned DNA
agctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacgagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtacatga

SEQ ID NO: 69 - DNA, 2081 bp, Homo sapiens
  misc_feature 1..2081: note = PREDICTED: Homo sapiens peptidyl arginine deiminase 4 (PADI4), transcript variant X1, mRNA
  source 1..2081: mol_type = unassigned DNA
agccagagggacgagctagcccgacgatggcccaggggacattgatccgtgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtacatga

SEQ ID NO: 70 - protein, 601 aa, Homo sapiens
  REGION 1..601: note = protein-arginine deiminase type-4 isoform X1 [Homo sapiens]
MAQGTLIRVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 71 - DNA, 2081 bp, Homo sapiens; identical to SEQ ID NO: 69
  misc_feature 1..2081: note = PREDICTED: Homo sapiens peptidyl arginine deiminase 4 (PADI4), transcript variant X1, mRNA
  source 1..2081: mol_type = unassigned DNA

SEQ ID NO: 72 - protein, 601 aa, Homo sapiens; identical to SEQ ID NO: 70
  REGION 1..601: note = protein-arginine deiminase type-4 isoform X1 [Homo sapiens]

SEQ ID NO: 73 - DNA, 1969 bp, Homo sapiens
  misc_feature 1..1969: note = PREDICTED: Homo sapiens peptidyl arginine deiminase 4 (PADI4), transcript variant X2, mRNA
  source 1..1969: mol_type = unassigned DNA
acagccagagggacgagctagcccgacgatggcccaggggacattgatccgtgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtacatga

SEQ ID NO: 74 - protein, 563 aa, Homo sapiens
  REGION 1..563: note = protein-arginine deiminase type-4 isoform X2 [Homo sapiens]
MAQGTLIRVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 75 - DNA, 1969 bp, Homo sapiens; identical to SEQ ID NO: 73
  misc_feature 1..1969: note = PREDICTED: Homo sapiens peptidyl arginine deiminase 4 (PADI4), transcript variant X2, mRNA
  source 1..1969: mol_type = unassigned DNA

SEQ ID NO: 76 - protein, 563 aa, Homo sapiens; identical to SEQ ID NO: 74
  REGION 1..563: note = protein-arginine deiminase type-4 isoform X2 [Homo sapiens]

SEQ ID NO: 77 - DNA, 1358 bp, Homo sapiens
  misc_feature 1..1358: note = PREDICTED: Homo sapiens peptidyl arginine deiminase 4 (PADI4), transcript variant X4, mRNA
  source 1..1358: mol_type = unassigned DNA
acagccagagggacgagctagcccgacgatggcccaggggacattgatccgtgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgattctatcaataggcccattttatagaagaagaaactgaggctcagagtggagaagttggaattcaaacccggccagtccggcttgggtccagattttggctatgtaactcgagggccccaaacagggggtatcagtggactggactcctttgggaacctggaagtgagcccccc

SEQ ID NO: 78 - protein, 397 aa, Homo sapiens
  REGION 1..397: note = protein-arginine deiminase type-4 isoform X4 [Homo sapiens]
MAQGTLIRVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMILSIGPFYRRRN

SEQ ID NO: 79 - DNA, 1358 bp, Homo sapiens; identical to SEQ ID NO: 77
  misc_feature 1..1358: note = PREDICTED: Homo sapiens peptidyl arginine deiminase 4 (PADI4), transcript variant X4, mRNA
  source 1..1358: mol_type = unassigned DNA

SEQ ID NO: 80 - protein, 397 aa, Homo sapiens; identical to SEQ ID NO: 78
  REGION 1..397: note = protein-arginine deiminase type-4 isoform X4 [Homo sapiens]

SEQ ID NO: 81 - DNA, 1842 bp, Homo sapiens
  misc_feature 1..1842: note = PREDICTED: Homo sapiens peptidyl arginine deiminase 4 (PADI4), transcript variant X6, mRNA
  source 1..1842: mol_type = unassigned DNA
acagccagagggacgagctagcccgacgatggcccaggggacattgatccgtgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatggaagaccccaggcctgcgctctgagtcctggcagccccttggctcaaggtgcccagggaaaacgacggaaggagtgtggggtccaaggtttgctaggacaaatggctttccgacaagcatctgtactgtgccccaagggaggggagagtggccctgcccacacagaaggcttcagaatgaaacttcagttaaactcaacaacttgtcccgagtctcgcttcctcctggccaagctgtgcgctgagcagtggagatgcagagatgctgttggattcacagtgtgtactcagccatggggtcgagttcatcctgtttcagcagcgttggggcgaaccagccagctgctaccgtgactttgccctctttctgctgctttcagtcctgcctggttcagtggctgtgtctactttgatgctccaagtataacaaaggccagattttctgatgccacaagtcctgctggacagacagacggacacctcggcaccattgttgtcccctttcccacagacaactttggatgaaggcaggcagcactggtccctcagctaagcacatctgtgtattccaactcaaatcactcagaatgaggccaggtgtggtggctgacacctgtaatcccagcagtttgggaggccaaggaggatggatcacctgag

SEQ ID NO: 82 - protein, 392 aa, Homo sapiens
  REGION 1..392: note = protein-arginine deiminase type-4 isoform X5 [Homo sapiens]
MAQGTLIRVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMEDPRPAL

SEQ ID NO: 83 - DNA, 1842 bp, Homo sapiens; identical to SEQ ID NO: 81
  misc_feature 1..1842: note = PREDICTED: Homo sapiens peptidyl arginine deiminase 4 (PADI4), transcript variant X6, mRNA
  source 1..1842: mol_type = unassigned DNA

SEQ ID NO: 84 - protein, 392 aa, Homo sapiens; identical to SEQ ID NO: 82
  REGION 1..392: note = protein-arginine deiminase type-4 isoform X5 [Homo sapiens]

SEQ ID NO: 85 - DNA, 1275 bp, Homo sapiens
  misc_feature 1..1275: note = PREDICTED: Homo sapiens peptidyl arginine deiminase 4 (PADI4), transcript variant X7, mRNA
  source 1..1275: mol_type = unassigned DNA
acagccagagggacgagctagcccgacgatggcccaggggacattgatccgtgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgcaatgacagccggcagatgcaccaggccctgcaggacttcctcagtgcccagcaggtgcaggcccctgtgaagctctattctgactggctgt

SEQ ID NO: 86 - protein, 386 aa, Homo sapiens
  REGION 1..386: note = protein-arginine deiminase type-4 isoform X6 [Homo sapiens]
MAQGTLIRVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMQ

SEQ ID NO: 87 - DNA, 1275 bp, Homo sapiens; identical to SEQ ID NO: 85
  misc_feature 1..1275: note = PREDICTED: Homo sapiens peptidyl arginine deiminase 4 (PADI4), transcript variant X7, mRNA
  source 1..1275: mol_type = unassigned DNA

SEQ ID NO: 88 - protein, 386 aa, Homo sapiens; identical to SEQ ID NO: 86
  REGION 1..386: note = protein-arginine deiminase type-4 isoform X6 [Homo sapiens]

SEQ ID NO: 89 - DNA, 1340 bp, Homo sapiens
  misc_feature 1..1340: note = PREDICTED: Homo sapiens peptidyl arginine deiminase 4 (PADI4), transcript variant X5, mRNA
  source 1..1340: mol_type = unassigned DNA
acagccagagggacgagctagcccgacgatggcccaggggacattgatccgtgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacggccctccagccaagaagaaatccacaggttcctccacatggcccctggaccctggggtagaggtgaccctgacgatgaaagtggccagtggtagcacaggcgaccagaaggttcagatttcatactacggacccaagactccaccagtcaaagctctactctacctcaccggggtggaaatctccttgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgcgtgatgattctatcaataggcccattttatagaagaagaaactgaggctcagagtggagaagttggaattcaaacccggccagtccggcttgcaatgacagccggcagatgcaccaggccctgcaggacttcctcagtgcccagcaggtgcaggcccctgtga

SEQ ID NO: 90 - protein, 397 aa, Homo sapiens; identical to SEQ ID NO: 78
  REGION 1..397: note = protein-arginine deiminase type-4 isoform X4 [Homo sapiens]

SEQ ID NO: 91 - DNA, 1340 bp, Homo sapiens; identical to SEQ ID NO: 89
  misc_feature 1..1340: note = PREDICTED: Homo sapiens peptidyl arginine deiminase 4 (PADI4), transcript variant X5, mRNA
  source 1..1340: mol_type = unassigned DNA

SEQ ID NO: 92 - protein, 397 aa, Homo sapiens; identical to SEQ ID NO: 78
  REGION 1..397: note = protein-arginine deiminase type-4 isoform X4 [Homo sapiens]

(19) United States
(12) Patent Application Publication
(10) Pub. No.: US 2025/0258175 A1
(43) Pub. Date: Aug. 14, 2025

(54) COMPOSITIONS AND METHODS FOR DIAGNOSING AND ASSESSING RHEUMATOID ARTHRITIS

(71) Applicant: Inova Diagnostics, Inc., San Diego, CA (US)

(72) Inventor: Michael Mahler, Bad Neuenahr (DE)

(21) Appl. No.: 19/184,462

(22) Filed: Apr. 21, 2025

Related U.S. Application Data
(63) Continuation of application No. 16/791,779, filed on Feb. 14, 2020, now Pat. No. 12,298,304.
(60) Provisional application No. 62/806,607, filed on Feb. 15, 2019.

(51) Int. Cl. G01N 33/573; G01N 33/58

(52) U.S. Cl. CPC G01N 33/573; G01N 33/58; G01N 2458/00; G01N 2800/105; G01N 2800/56

(57) ABSTRACT

The present disclosure relates to the use of anti-PAD IgA as a clinical biomarker for diagnostic and prognostic information in rheumatoid arthritis (RA) patients. The disclosure further provides methods and compositions for the detection anti-PAD IgA in a biological sample.

This application claims the benefit of U.S. Provisional Application No. 62/806,607 filed Feb. 15, 2019, the entire contents of which are incorporated herein by reference.

This application incorporates herein by reference a Sequence Listing as an ASCII text file entitled “13510-034-999_SEQ_LISTING” created on Feb. 12, 2020, and having a size of 385,711 bytes.

### FIELD

The present disclosure relates to the field of molecular biology and more specifically to methods for detecting anti-PAD IgA in the serum of rheumatoid arthritis (RA) patients.

### BACKGROUND

Rheumatoid Arthritis (RA) is a chronic autoimmune disease characterized by inflammation, pain and subsequent damage to synovial-lined joints. Unlike other arthritis conditions, RA is a systemic disease that can affect other organ systems including but not limited to the cardiovascular system, the respiratory system and musculature. While the exact pathogenesis of the disease is unknown, RA is characterized by the production of antibodies to self-proteins (autoantibodies) by the immune system. The most common autoantibodies implicated in RA include rheumatoid factor (RF) and anti-citrullinated protein antibodies (ACPAs), which are part of the classification criteria for this disease. ACPAs are a hallmark amongst serologic factors detected in RA patients, and as such, serve as valuable diagnostic and prognostic markers. Aletaha D. et al., Ann. Rheum. Dis. 2010, 69, 1580-1588); Taylor et al., Autoimmune Dis; 2011:815038 (2011). However, clinical heterogeneity of RA precludes the use of ACPAs and RF alone as reliable biomarkers. Patients with erosive disease require more aggressive treatment in the early phase of the disease to prevent joint damage. More precise biomarkers that specifically identify sufferers of RA and disease severity are needed.

Peptidylarginine deiminases (PAD) s are calcium-dependent enzymes that play a central role in generating autoantigens in RA through the conversion of arginine residues to citrulline, process known as citrullination. Beyond ACPA and RF, autoantibodies which target the PAD enzymes, have also been described in RA, see for example, Takizawa et al., Scand. J. Rheumatol. 3:212-215 (2005); Roth et al., Clin. Exp. Rheumatol. 1:12-18 (2006); Halvorsen et al., Ann. Rheumatol. Dis. 67:414-417 (2008); Zhao et al., J. Rheumatol., 35:969-974 (2008); Darrah et al., Sci. Trans. Med., 5 (186): 186ra65 (2013); Darrah et al., Front. Immunol., 9:2696 (2018). As such, PADs appear to play a central role in RA pathogenesis.

Thus, there exists a need for additional biomarkers for the diagnosis of RA and assessment of disease severity, including erosive conditions. The present disclosure satisfies this need and provides related advantages as well.

### SUMMARY

In some embodiments, the present disclosure provides a method of diagnosing rheumatoid arthritis (RA). The method includes: (a) contacting a biological sample from a subject suspected of having RA with a peptidyl arginine deiminase (PAD) or an antigenic fragment thereof, and (b) detecting the presence of an anti-PAD IgA in the biological sample, wherein the presence of the anti-PAD IgA is indicative of RA.

In some embodiments, the present disclosure provides a method of assessing disease severity in a subject having RA. The method includes: (a) contacting a biological sample from a subject having RA with a PAD or an antigenic fragment thereof, and (b) detecting the presence of an anti-PAD IgA in the biological sample, wherein the presence of the anti-PAD IgA is indicative of severity of RA.

In some embodiments, the biological sample includes whole blood, plasma, serum, synovial fluid or sputum. In some embodiments, the biological sample includes serum or plasma.

In some embodiments, the present disclosure provides a method of assessing disease severity, wherein the severity of RA includes the presence of joint erosion. In other embodiments, the severity of RA includes severe joint erosion. In other embodiments, anti-PAD IgA levels correlate with extent of joint erosion.

In some embodiments, the extent of joint erosion includes reduced mobility. In other embodiments, the reduced mobility includes a disability index of approximately 3.

In some embodiments, the PAD or antigenic fragment thereof used in the method of diagnosing RA or assessing disease severity is selected from the group consisting of PAD2, PAD3 and PAD4.

In some embodiments, the PAD or antigenic fragment thereof includes an amino acid sequence selected from an even numbered SEQ ID NO within SEQ ID NOS: 2-92 or an amino acid sequence comprising at least six consecutive amino acids selected from an even numbered SEQ ID NO within SEQ ID NOS: 2-92. In some embodiments, the antigenic fragment includes from 6-120, 12-100, 18-80, 24-60, 30-50 or 35-45 amino acid residues.

In some embodiments, the PAD or antigenic fragment thereof is obtained by a method comprising isolation from a natural source, chemical synthesis or recombinant expression.

In some embodiments, detection includes an immunoassay. In some embodiments, the immunoassay is selected from the group consisting of a fluorescent immunosorbent assay (FIA), a chemiluminescent immunoassay (CIA), a radioimmunoassay (RIA), multiplex immunoassay, a protein/peptide array immunoassay, a solid phase radioimmunoassay (SPRIA), an indirect immunofluorescence assay (IIF), an enzyme linked immunosorbent assay (ELISA) and a particle based multianalyte test (PMAT), or a Dot Blot assay.

In some embodiments, the method described herein can be performed by (a) contacting the anti-PAD IgA with a detection probe specific for the anti-PAD IgA and (b) detecting specific binding of the detection probe. In some embodiments, the detection probe is specific to PAD. In other embodiments, the detection probe includes an antibody or functional fragment. In some embodiments, the functional fragment is anti-IgA. In some embodiments, the detection probe is a reporter tag.

In some embodiments, the reporter tag includes a label. In some embodiments, the label is selected from the group consisting of a fluorophore, enzyme, chemiluminescent moiety, radioactive moiety, organic dye and small molecule. In some embodiments, the label is a fluorescent label. In some embodiments, the fluorescent label is phycoerytherin (PE).

In some embodiments, the reporter tag includes a ligand or particle. In some embodiments, the ligand includes biotin. In some embodiments, the particle includes a nanoparticle.

In other embodiments, the reporter tag is a ligand or particle. In some embodiments, the ligand is biotin and the particle is a nanoparticle.

In some embodiments, the present disclosure provides a kit. The kit includes: (a) a PAD, or antigenic fragment thereof; (b) a detection probe specific to anti-PAD IgA, and (c) a solid support.

In some embodiments, the kit further includes a label. In some embodiments, the kit includes a label selected from the group consisting of a fluorophore, enzyme, chemiluminescent moiety, radioactive moiety, organic dye and small molecule.

In some embodiments, the kit includes a positive control. In some embodiments, the positive control includes an anti-PAD IgA.

In some embodiments, the kit further includes one or more ancillary reagents. In some embodiments, the one or more ancillary reagents is selected from the group consisting of an incubation buffer, a wash buffer, a detection buffer and a detection instrument.

In some embodiments, the kit includes a PAD or antigenic fragment thereof selected from the group consisting of PAD2, PAD3 and PAD4.

In some embodiments, the PAD or antigenic fragment thereof in the kit includes an amino acid sequence selected from an even numbered SEQ ID NO within SEQ ID NOS: 2-92 or an amino acid sequence comprising at least six consecutive amino acids selected from an even numbered SEQ ID NO within SEQ ID NOS: 2-92.

In some embodiments, the kit contains an antigenic fragment including from 6-120, 12-100, 18-80, 24-60, 30-50 or 35-45 amino acid residues.

In some embodiments, the detection probe includes an antibody or functional fragment thereof. In some embodiments, the antibody or functional fragment thereof includes anti-IgA.

In some embodiments, the detection probe includes a reporter tag. In some embodiments, the reporter tag includes a label. In some embodiments, the label is selected from the group consisting of a fluorophore, enzyme, chemiluminescent moiety, radioactive moiety, organic dye and small molecule.

In some embodiments, the label is a fluorescent label. In some embodiments, the fluorescent label is (PE).

In some embodiments, the reporter tag includes a ligand or particle. In some embodiments, the ligand includes biotin. In some embodiments, the particle includes a nanoparticle.

In some embodiments, the solid support is selected from the group consisting of a bead, sphere, particle, membrane, chip, slide, plate, well and test tube. In some embodiments, the bead, sphere or particle includes micrometer or nanometer dimensions.

In some embodiments, the membrane is selected from the group consisting of nitrocellulose, nylon, polyvinylidene fluoride (PVDF) and polyvinylidene difluoride.

In some embodiments, the PAD or antigenic fragment thereof is conjugated to the solid support.

### BRIEF DESCRIPTION OF THE DRAWINGS

FIG. 1 shows association of anti-PAD4 IgA with joint erosion status. Results are expressed in Median Fluorescence Intensity (MFI). P-value of the Mann-Whitney analysis is shown in red (p-value<0.05 considered significant). Median MFI for the subgroup and number of patients and % are shown. Red dashed line represents the preliminary cut-off.

FIG. 2 shows association of anti-PAD2 IgA with joint erosion status. Results are expressed in Median Fluorescence Intensity (MFI). P-value of the Mann-Whitney analysis is shown in red (p-value<0.05 considered significant). Median MFI for the subgroup and number of patients and % are shown. Red dashed line represents the preliminary cut-off.

FIG. 3 shows a receiver operating characteristic (ROC) analysis of anti-PAD2 IgA (square) and anti-PAD4 IgA (triangle) illustrating the discrimination between erosive and non-erosive disease in RA patients. Area Under the Curve (AUC) for each marker is shown in the legend.

FIG. 4 shows SEQ ID NO:1 which includes the mRNA nucleotide sequence of a human wild-type PAD2. The accession number for SEQ ID NO:1 is NM_007365.3.

FIG. 5 shows sequence number-2 which includes the amino acid sequence of a human wild-type PAD2. The accession number for SEQ ID NO:2 is NP_031391.2. SEQ ID NO:2 is the polypeptide encoded by SEQ ID NO:1.

FIG. 6 shows SEQ ID NO:3 which includes the mRNA nucleotide sequence of human PAD2 transcript variant X2. The accession number for SEQ ID NO:3 is XM_017000148.2. SEQ ID NO:3 is a transcript variant of SEQ ID NO:1.

FIG. 7 shows SEQ ID NO:4 which includes the amino acid sequence of a human PAD2 isoform X1. The accession number for SEQ ID NO:4 is XP_016855637.1. SEQ ID NO:4 is the polypeptide encoded by SEQ ID NO:3.

FIG. 8 shows SEQ ID NO:5 which includes the mRNA nucleotide sequence of a human wild-type PAD3. The accession number for SEQ ID NO:5 is NM_016233.2.

FIG. 9 shows SEQ ID NO:6 which includes the amino acid sequence of a human wild-type PAD3. The accession number for SEQ ID NO:6 is NP_057317.2. SEQ ID NO:6 is the polypeptide encoded by SEQ ID NO:5.

FIG. 10 shows SEQ ID NO:21 which includes the mRNA nucleotide sequence of a human PAD3 transcript variant X1. The accession number is XM_011541571.2. SEQ ID NO:21 is a transcript variant of SEQ ID NO:5.

FIG. 11 shows SEQ ID NO:22 which includes the amino acid sequence of a human PAD3 isoform X1. The accession number is XP_011539873.1. SEQ ID NO:22 is the polypeptide encoded by SEQ ID NO:21.

FIG. 12 shows SEQ ID NO:37 which includes the mRNA nucleotide sequence of a human PAD3 transcript variant X2. The accession number is XM_017001463.1. SEQ ID NO: 37 is a transcript variant of SEQ ID NO:5.

FIG. 13 shows SEQ ID NO:38 which includes the amino acid sequence of a human PAD3 isoform X2. The accession number is XP_016856952.1. SEQ ID NO:38 is the polypeptide encoded by SEQ ID NO:37.

FIG. 14 shows SEQ ID NO:39 which includes the mRNA nucleotide sequence of a human PAD3 transcript variant X2. The accession number is XM_017001463.1: c. SEQ ID NO: 39 is a transcript variant of SEQ ID NO:5.

FIG. 15 shows SEQ ID NO:40 which includes the mRNA nucleotide sequence of a human PAD3 transcript variant X2. The accession number is XM_017001463.1: c. SEQ ID NO: 40 is a transcript variant of SEQ ID NO:5.

FIG. 16 shows SEQ ID NO:49 which includes the mRNA nucleotide sequence of a human PAD3 transcript variant X3. The accession number is XM_011541572.2. SEQ ID NO: 49 is a transcript variant of SEQ ID NO:5.

FIG. 17 shows SEQ ID NO:50 which includes the amino acid sequence of a human PAD3 isoform X3. The accession number is XP_011539874.1. SEQ ID NO:50 is the polypeptide encoded by SEQ ID NO:49.

FIG. 18 shows SEQ ID NO:59 which includes the mRNA nucleotide sequence of a human PAD3 transcript variant X3. The accession number is XM_011541572.2: c. SEQ ID NO: 59 is a transcript variant of SEQ ID NO:5.

FIG. 19 shows SEQ ID NO:61 which includes the mRNA nucleotide sequence of a human wild-type PAD4. The accession number is NM_012387.3.

FIG. 20 shows SEQ ID NO:62 which includes the amino acid sequence of a human wild-type PAD4. The accession number is NP_036519.2. SEQ ID NO:62 is the polypeptide encoded by SEQ ID NO:61.

FIG. 21 shows SEQ ID NO:67 which includes the mRNA nucleotide sequence of a human PAD4 transcript, variant X3. The accession number is XM_011541152.1. SEQ ID NO: 67 is a transcript variant of SEQ ID NO:61.

FIG. 22 shows SEQ ID NO:68 which includes the mRNA nucleotide sequence of a human PAD4 transcript variant X8. The accession number is XM_011541157.1. SEQ ID NO:68 is a transcript variant of SEQ ID NO:61.

FIG. 23 shows receiver operating characteristic (ROC) analysis of anti-PAD4 IgA (blue), anti-PAD4 IgG (grey) and anti-PAD4 IgM (red), illustrating the discrimination between RA and controls. Area Under the Curve (AUC) for each marker is shown in the legend.

FIG. 24 shows receiver operating characteristic (ROC) analysis of anti-PAD4 IgA (blue), anti-PAD4 IgG (grey) and anti-PAD4 IgM (red), illustrating discrimination for RA erosive disease. Area Under the Curve (AUC) for each marker is shown in the legend.

FIG. 25 shows association of anti-PAD4 IgA with joint erosion status. Results are expressed in Median Fluorescence Intensity (MFI). P-value of the Mann-Whitney analysis is shown in the graph (p-value <0.05 considered significant).

FIG. 26 shows association of anti-PAD4 IgG with joint erosion status. Results are expressed in Median Fluorescence Intensity (MFI). P-value of the Mann-Whitney analysis is shown in the graph (p-value <0.05 considered significant).

FIG. 27 shows association of anti-PAD4 IgM with joint erosion status. Results are expressed in Median Fluorescence Intensity (MFI). P-value of the Mann-Whitney analysis is shown in the graph (p-value <0.05 considered significant).

### DETAILED DESCRIPTION

The present disclosure is based, in part, on the discovery that anti-PAD IgA serves as a diagnostic biomarker for RA and also as an indicator of disease severity for RA. Thus, the present disclosure benefits RA patients by providing a new biomarker which can indicate the presence of RA, disease severity, including erosive arthritis, and facilitate the early detection of RA and treatment escalation. Such benefits further enable at risk or early-stage RA patients to reduce or prevent disease progression and related erosive conditions such as joint erosion.

It must be noted that, as used in this specification and the appended claims, the singular forms “a”, “an” and “the” also include plural referents unless the content clearly dictates otherwise.

It must also be noted that, as used in this specification and the appended claims, where a range of numeric values is provided, it is understood that the ranges are inclusive of the numbers defining the range. It is also understood that each intervening integer within the recited range as well as fractions thereof, including for example, every tenth of a unit of a selected intervening integer or a lower limit of the recited range is intended to be included within the disclosure, unless the context clearly dictates otherwise.

As used herein, the terms “comprises,” “comprising,” “includes,” “including,” “has,” “having,” “contains,” “containing,” and any variations thereof, are intended to cover a non-exclusive inclusion, such that a process, method, product-by-process, or composition of matter that includes has or contains an element or list of elements, does not include only those elements but can include other elements not expressly listed or inherent to such process, method, product-by-process, or composition of matter.

The present disclosure provides a method of diagnosing RA. The method includes: (a) contacting a biological sample from a subject suspected of having RA with a PAD or an antigenic fragment thereof, and (b) detecting the presence of anti-PAD IgA in the biological sample, wherein the presence of the anti-PAD IgA is indicative of RA.

The present disclosure also provides a method of assessing disease severity in a subject having RA. The method includes: (a) contacting a biological sample from a subject having RA with PAD or an antigenic fragment thereof, and (b) detecting the presence of anti-PAD IgA in the biological sample, wherein the presence of the anti-PAD IgA is indicative of severity of RA. Disease severity can be the presence of joint erosion, including assessing the extent of joint erosion.

The term “autoantibody” refers to an immunoglobulin directed against a constituent of tissue of the subject that produces the autoantibody. The term is intended to include an antibody produced by a subject's immune system that is directed against one or more of the subject's own polypeptides or antigens. Accordingly, autoantibodies can be produced by a subject's immune system when the immune system fails to distinguish, in whole or in part, between self and non-self tissue constituents. As provided herein an autoantibody directed to or specific for PAD having an IgA isotype is a beneficial biomarker for diagnosing RA, assessing disease severity of RA and/or diagnosing or determining the severity of joint erosion.

As used herein, the terms “anti-PAD IgA” when used in reference to an autoantibody is intended to mean an IgA autoantibody directed against PAD or an antigenic fragment thereof. An IgA autoantibody is distinguishable from other antibody classes including, for example, gamma (IgG), mu (IgM), delta (IgD) and epsilon (IgE) antibody classes based in part on the constant region sequence and/or structure and other characteristics well-known in the art. IgA includes, for example, IgA1 and IgA2 subclasses as well as secretory IgA. IgA1 and IgA2 exist in monomer and dimer configurations and can form polymers with IgM. The term “anti-PAD IgA” is intended to include all IgA subclasses as well as the monomer, dimer and polymer configurations.

The presence of increased anti-PAD IgA in a subject compared to a healthy control individual can be indicative of the presence of RA, the severity of disease or the risk of developing RA. Accordingly, a measurable increase in an autoantibody to PAD having an IgA isotype, and any IgA subtypes, is used to diagnose RA, determine the severity of RA and/or diagnose or determine the severity levels of joint erosion. Exemplary methods for detection and comparison of anti-PAD IgA levels to a control are provided herein and described further below.

In some embodiments, detection of an increased level of anti-PAD IgA compared to a healthy control individual is indicative of a subject having RA. In some embodiments, following diagnosis of RA using the compositions and methods provided herein, the presence of RA can be further corroborated based on a variety of symptoms associated with the onset or presence of RA. Clinical symptoms associated with RA include, for example, pain and swelling of small and large bilateral joints, palindromic onset, monoarticular presentation, and extra-articular synovitis, like tenosynovitis and bursitis, polymyalgic-like onset and other symptoms including malaise, weight loss, fatigue, fever and disability. Grassi et al., Eur. J. Radiol., Suppl 1:S 18-24 (1998); Aletaha and Smolen, JAMA, 320(13):1360-1372 (2018).

In some embodiments, detection of an increased level of anti-PAD IgA in a subject compared to a healthy control is indicative of having severe RA. In other embodiments, detection of an increased level of anti-PAD IgA in a subject compared to an RA subject without an increased level of anti-PAD IgA, is indicative of having severe RA. In some embodiments, having severe RA is considered by the degree of joint erosion or the risk of radiographic progression as determined by methods in the art. Detection of an increased level of anti-PAD IgA in a subject compared to a healthy control or compared to an RA subject without an increased level of anti-PAD IgA is indicative that the subject has a higher probability of having more progressed RA wherein joint erosion is severe. In some embodiments, a subject having increased anti-PAD IgA can be more than 5%, more than 10%, more than 15%, more than 20%, more than 25%, more than 30%, more than 35%, more than 40%, more than 45%, more than 50%, more than 60%, more than 70%, more than 80% or more than 90% likely to have more progressed RA where severe joint erosion is present. In other embodiments, a subject having increased anti-PAD IgA can be more than 2-fold, more than 3-fold, more than 4-fold, more than 5-fold, more than 6-fold, more than 7-fold, more than 8-fold, more than 9-fold, or more than 10-fold likely to have more progressed RA where severe joint erosion is present.

In severe RA, joint erosion occurs when there is loss of bone and cartilage in the joint. Severity of joint erosion can be determined by, for example, the Sharp score method. See Sharp, Arthritis Rheum., 32:221-229 (1989); Brower, Arthritis Rheum., 33:316-324 (1990). The Sharp score assesses joints for narrowness and erosions, based upon radiographic images. Erosion scores range from 0-3.5 and joint space narrowing scores range from 0-4. A score of 0 indicates a normal joint with no narrowing or erosions and a score of 3.5-4 indicates an abnormal joint with erosions and narrowing. In some embodiments of the present disclosure, joint erosion in a subject is determined by use of the Sharp score.

In other embodiments, having severe RA is determined by the Health Assessment Questionnaire (HAQ) Disability Index (DI). Fries et al., Arthritis Rheum, 23(2):137-145 (1980); Bruce and Fries, Health Qual Life Outcomes, 1(1):20 (2003). The HAQ assesses physical ability in 8 sections including dressing, arising, eating, walking, hygiene, reach, grip and activities. Performing each session is allotted a score ranging from 0 (without any difficulty) to 3 (unable to do). The scores of the 8 sections are summed and divided by 8 to produce the DI. The DI, which ranges from 0 to 3, predicts disability, with a person able to complete a task without any difficulty (DI of 0), with some difficulty (DI of 1), with much difficulty (DI of 2), or unable to do (DI of 3).

Thus, in some embodiments, the presence of increased anti-PAD IgA in a subject compared to a healthy control individual is indicative of the presence of joint erosion. In other embodiments, detection of an increased level of anti-PAD IgA in a subject compared to an RA subject without an increased level of anti-PAD IgA is indicative of having joint erosion. In other embodiments, the presence of increased anti-PAD IgA compared to a healthy individual is indicative of the presence of severe joint erosion. In some embodiments, detection of an increased level of anti-PAD IgA compared to an RA subject without having an increased level of anti-PAD IgA is indicative of having severe joint erosion. In another embodiment, the presence of anti-PAD IgA is indicative of a DI of 2 or more, or of 3 or more.

In alternative embodiments, having joint erosion includes having worse radiographic joint damage. In some embodiments, having severe joint erosion includes having worse baseline radiographic joint damage. Accordingly, detection of an increased level of anti-PAD IgA in a subject compared to a healthy control individual is indicative of having worse radiographic joint damage and/or worse baseline radiographic joint damage. In other embodiments, detection of an increased level of anti-PAD IgA in a subject compared to a RA subject without having an increased level of anti-PAD IgA is indicative of having worse radiographic joint damage and/or worse baseline radiographic joint damage. One skilled in the art will recognize that methods for determining and assessing radiographic joint damage are well known in the art. Additionally, those skilled in the art will recognize and employ suitable techniques to quantify radiographic joint damage. As a non-limiting example, the Sharp scoring method can be used.

In some embodiments, detection of an increased level of anti-PAD IgA compared to a healthy control individual indicates that the subject is at risk of developing clinical symptoms of RA. In some embodiments, a subject can be at risk of developing clinical symptoms of RA within less than 3 months, less than 6 months, less than 9 months, less than 12 months, less than 18 months, less than 2 years, less than 3 years, less than 4 years, less than 5 years, less than 6 years, less than 7 years, less than 8 years, less than 9 years, less than 10 years, less than 12 years, less than 14 years, or less than 16 years from the determination of the increased anti-PAD IgA level.

In some embodiments, the presence of an increased level of anti-PAD IgA compared to a healthy control individual indicates that the subject is more than 5%, more than 10%, more than 15%, more than 20%, more than 25%, more than 30%, more than 35%, more than 40%, more than 45%, more than 50%, more than 60%, more than 70%, or more than 80% or more than 90% likely to develop clinical symptoms of RA within 5 years following the determination of increased anti-PAD IgA. In some embodiments, the presence of an increased level of anti-PAD IgA can indicate that the subject is more than 2-fold, more than 3-fold, more than 4-fold, more than 5-fold, more than 6-fold, more than 7-fold, more than 8-fold, more than 9-fold, or more than 10-fold likely to develop clinical symptoms of RA within 5 years following determination of increased anti-PAD IgA level compared to a healthy control individual.

Anti-PAD IgA can be detected in a variety of different biological samples obtained from a subject. Such samples include, for example, solid tissue and biological fluids. As used herein, the term “biological sample” refers to any specimen from the body of an organism that can be used for analysis or diagnosis. In the context of the present disclosure, a biological sample obtained from a subject can be any sample that contains or is suspected to contain autoantibodies and encompasses any material in which an anti-PAD autoantibody can be detected. For example, a biological sample can include a liquid sample such as whole blood, plasma, serum, synovial fluid, amniotic fluid, sputum, pleural fluid, peritoneal fluid, central spinal fluid, urine, saliva, tears or other body fluid that contains autoantibodies. A biological sample can also include a solid tissue sample such as bone marrow, tissue, buccal or other solid or semi-solid aggregate of cells.

In some embodiments, anti-PAD IgA is detected in whole blood, plasma, serum, synovial fluid or sputum. In some embodiments of the present disclosure, the level of anti-PAD IgA is detected. In other embodiments, anti-PAD IgA-PAD complex can be formed using the compositions and methods described herein and an anti-PAD IgA in the complex can be detected. Accordingly, the disclosure provides compositions that include an anti-PAD IgA-PAD complex.

The biological samples of this disclosure can be obtained from any organism including, for example, mammals such as humans, primates such as monkeys, chimpanzees, orangutans and gorillas, cats, dogs, rabbits, farm animals such as cows, horses, goats, sheep and pigs, and rodents such as mice, rats, hamsters and guinea pigs.

In some embodiments, the biological sample can be a plurality of samples. In some embodiments the plurality of samples can be obtained periodically over the course of more than 12 hours, more than 1 day, more than 2 days, more than 3 days, more than 4 days, more than 5 days, more than 6 days, more than 7 days, more than 10 days, more than 14 days, more than 3 weeks, more than 1 month, more than 2 months, more than 3 months, more than 4 months, more than 5 months, more than 6 months, more than 9 months, more than 12 months, more than 18 months, more than 24 months, more than 30 months, more than 3 years months, more than 4 years or more than 5 years.

In some embodiments, the samples of the present disclosure can be collected and processed fresh. In other embodiments, the samples of the present disclosure can be frozen, stored and processed at a later date.

In some embodiments, the present disclosure provides a method of determining the level of anti-PAD IgA in a subject to determine if that subject has RA, severe RA or joint erosion, including severe joint erosion. It is noted that, as used herein, the terms “subject,” “organism,” “individual” or “patient” are used as synonyms and interchangeably, and refer to a vertebrate mammal. Mammals include humans, primates such as monkeys, chimpanzees, orangutans and gorillas, cats, dogs, rabbits, farm animals such as cows, horses, goats, sheep and pigs, and rodents such as mice, rats, hamsters and guinea pigs. The subjects of this disclosure can include healthy subjects, asymptomatic subjects, and diseased subjects.

In some embodiments, the diseased subjects can suffer from any disease associated with aberrant anti-PAD IgA levels. It is noted that the term “aberrant anti-PAD IgA levels” refers to anti-PAD IgA levels in a sample that measurably deviate from the median anti-PAD IgA levels found in a population of healthy subjects. In some embodiments, the aberrant anti-PAD IgA levels can be higher than the median anti-PAD IgA levels. In some embodiments, the aberrant anti-PAD IgA levels can be lower than the median anti-PAD IgA levels.

In some embodiments, the healthy subjects can have never suffered from a certain disease. In some embodiments, the healthy subjects can be previously diseased. In some embodiments, the healthy subjects can be undergoing a routine medical checkup. In some embodiments, the healthy subjects can be members of a control group in, for example, a clinical trial. In some embodiments, the healthy subjects can be at risk of contracting a disease, as determined by the presence of certain risk factors that are well known in the art. Such risk factors include, without limitation, a genetic predisposition, a personal disease history, a familial disease history, a lifestyle factor, an environmental factor, a diagnostic indicator, and the like.

In some embodiments, the subject can be asymptomatic. Asymptomatic subjects include healthy subjects who have essentially no risk or only a low risk of developing RA (e.g., there is a less than 10%, less than 5%, less than 3%, or less than 1% probability that the asymptomatic patient will develop RA over the following five year period). Asymptomatic subjects further include healthy subjects who have a high risk of developing RA (e.g., there is a greater than 50%, greater than 70%, greater than 90%, or greater than 95% probability that the asymptomatic patient will develop RA over the following five year period). Asymptomatic subjects further include diseased subjects, who can display mild early diagnostic indicators of RA, but who are otherwise disease or complaint free (e.g., no synovial joint pain, no systemic inflammatory disorder). In some embodiments, the asymptomatic patient can be an arthralgia patient.

In some embodiments, the subject can have RA. In some embodiments, the subject can have RA with joint pain. In some embodiments, the subject can have RA with a systematic inflammatory disorder. In some embodiments, the subject can have juvenile idiopathic arthritis (JIA). In some embodiments, the subject can have a pre-RA syndrome. In some embodiments, the pre-RA syndrome can be arthralgia.

In some embodiments, the subject can be suspected of having RA. As used herein, a subject can be “suspected of having RA” as determined by the presence of certain risk factors that are well known in the art. Such risk factors include, without limitation, a genetic predisposition, a personal disease history, a lifestyle factor, an environmental factor, a diagnostic indicator and the like.

In some embodiments, the subject can be at risk of developing RA. In some embodiments, the subject can have a genetic predisposition for developing RA or a family history of RA or other autoimmune diseases. In some embodiments, the subject can be exposed to certain lifestyle factors (e.g., smoking cigarettes) promoting the development of RA or the subject can show clinical disease manifestations of RA. In some embodiments, the subject can be a patient who is receiving a clinical workup to diagnose RA or to assess the risk of developing RA.

In some embodiments, the subjects can have anti-PAD IgA present, e.g., in their blood or another bodily tissue or fluid, (anti-PAD IgA-positive subjects). In some embodiments, the subjects can have elevated anti-PAD IgA levels, e.g., in their blood or another bodily tissue or fluid, relative to normal healthy subjects. In some embodiments, the subjects can have no anti-PAD IgA present, e.g., in their blood or another bodily tissue or fluid (anti-PAD IgA-negative subjects).

In some embodiments, the subjects can have anti-PAD IgA present, e.g., in their blood or another tissue or bodily fluid, (anti-PAD IgA-positive subjects) or the subjects can have elevated anti-PAD IgA levels, e.g., in their blood or another tissue or bodily fluid, relative to normal healthy subjects. In some embodiments, the subjects can be negative for anti-PAD IgA.

In some embodiments, the subject can be treatment naïve. In some embodiments, the subject can be undergoing treatments for RA (e.g., drug treatments). In some embodiments, the subject can be in remission. In some embodiments, the remission can be drug-induced. In some embodiments, the remission can be drug-free.

In some embodiments, the subject can be an animal model for RA. In some embodiments, the animal model can be a mouse, rabbit, or primate model of RA. In some embodiments, the animal model can involve inducing anti-PAD IgA responses by immunizing or vaccinating an animal with PAD.

It should be noted that the terms “healthy control individual,” “healthy subjects,” and grammatical equivalents herein are used interchangeably and refer to subjects who do not have increased anti-PAD IgA levels, RA or joint erosion above baseline or a standard known or determined to represent non-RA subjects.

The baseline or standard which determines or defines a subject as a non-RA subject is the reference interval. In diagnostic or health-related fields, the reference interval is a range of values observed in the reference subjects, which can be healthy control individuals, designated by specific percentiles. The reference interval can be any range of values as determined by those having skill in the art. See CLSI, “How to define and determine reference intervals in the clinical laboratory: approved guideline,” C28:A2 (2000). In some cases, the reference interval can be stringent or less stringent depending on the specific analyte being measured or disease being studied. A person having skill in the art will understand the appropriate stringency to use when determining the reference interval. Thus, in some embodiments, the reference interval can be set at the 95th percentile. In order to increase specificity and decrease sensitivity, e.g. increase stringency, a higher cut-off can be used such as the 96th percentile or the 97th, or the 98^{th}, or the 99th.

In the present disclosure, anti-PAD IgA can be considered increased in a subject if anti-PAD IgA levels are at least above the 95th percentile relative to anti-PAD IgA levels in healthy control subjects. In other embodiments, anti-PAD IgA can be considered increased in a subject if anti-PAD IgA levels are above the 96^{th}, 97^{th}, 98^{th} or 99^{th} percentile. A subject of the present disclosure with anti-PAD IgA levels at or above any of the disclosed reference intervals is considered to have RA and joint erosion

In some embodiments, the presence of anti-PAD IgA can be based on a comparison of signal against background in a healthy subject. In some embodiments, the presence of anti-PAD IgA can be increased or decreased relative to an average or median anti-PAD IgA level observed in a population of healthy subjects. In some embodiments, anti-PAD IgA can be absent in healthy subjects. In some embodiments, anti-PAD IgA level cannot be detected above the noise of the respective assay used to determine anti-PAD IgA level. In some embodiments, anti-PAD IgA can be considered present in a sample if an anti-PAD IgA level can be detected above the noise of the respective assay used to determine an anti-PAD IgA level. In some embodiments, anti-PAD IgA can be considered increased in a sample if the signal in an anti-PAD IgA detection assay is at least two standard deviations above noise such as the average or mean signal for control samples. In some embodiments, anti-PAD IgA can be considered present in a sample if the level of anti-PAD IgA exceeds a predetermined threshold level. An anti-PAD IgA threshold level can be determined by a skilled artisan, such as a clinical physician, based on a variety of factors, such as the specific objectives of a clinical trial or the diagnostic and prognostic significance of a certain anti-PAD IgA level or the results of another diagnostic test for RA that does not involve the detection of anti-PAD IgA levels.

In some embodiments, the present disclosure provides a polypeptide including a PAD or antigenic fragment thereof. The PAD can be used in the methods provided herein or included in the kits provided herein.

As used herein, the term “peptidyl arginine deiminase” or “PAD,” also known as PADI, refers to a family of enzymes that catalyze the post-translational modifications of protein arginine residues by deimination or demethylimination to produce citrulline (Wang and Wang, Biochim. Biophys. Acta., 1829:1126-35 (2013)). Five isotypes of PADs have been identified in humans and include PAD1, PAD2, PAD3, PAD4 and PAD6. All of such PAD polypeptides, PAD1, PAD2, PAD3, PAD4 and PAD6 are included within the meaning of the term “PAD” as it is used herein.

As used herein, the term “peptidyl arginine deiminase 2” or “PAD2,” also known as PADI2, PAD-H19 and PDI2, refers to a member of the PAD family of enzymes. PAD2 is abundantly expressed in secretory glands, brain, uterus, spleen, pancreas and skeletal muscle. Known substrates of PAD2 include myelin basic protein, vimentin and macrophages. See Vossenaar et al., Annals of the Rheumatic Diseases, 63:373-81 (2004); Watanbe et al., Biochim Biophys Acta., 966:375-383 (1988); Watanabe et al., J. Biol Chem., 264:15255-15260 (1989); Nagata et al., Experientia, 46:72-74 (1990); Urano et al., Am J Dermatopathol., 12 (3): 249-55 (1990), Vossenaar et al., Arthritis and Rheum., 48:2489-2500 (2003). Approximately 726 coding single nucleotide polymorphisms (SNP) have been identified for PAD2 and at least 184 known orthologs (see, for example, NCBI Gene ID: 11240). The term “PAD2” includes all of such PAD2 variants and PAD2 orthologs. An exemplary human PAD2 (hPAD2) nucleotide sequence can be found in GenBank under GenBank Accession number NM_007365 (SEQ ID NO: 1) and encodes an exemplary human PAD2 having the amino acid sequence found under found under GenBank Accession number NP_031391 (SEQ ID NO:2). The GenBank Accession numbers and GenBank GI numbers of this PAD2 and other exemplary PAD2 enzymes can be found below in Table 1. All of such PAD2 polypeptides and variants thereof are included within the meaning of the term “PAD2” as it is used herein.

In some embodiments, a PAD2, or antigenic fragment thereof, includes the amino acid in SEQ ID NO:2, of a mature human PAD2 (hPAD2; amino acids 25-665 of NCBI Accession Number NP_031391; GI: 122939159), or naturally occurring variants thereof:

SEQ ID NO: 2
MLRERTVRLQYGSRVEAVYVLGTYLWTDVYSAAPAGAQTFSLKHSEHVW
VEVVRDGEAEEVATNGKQRWLLSPSTTLRVTMSQASTEASSDKVTVNYY
DEEGSIPIDQAGLFLTAIEISLDVDADRDGVVEKNNPKKASWTWGPEGQ
GAILLVNCDRETPWLPKEDCRDEKVYSKEDLKDMSQMILRTKGPDRLPA
GYEIVLYISMSDSDKVGVFYVENPFFGQRYIHILGRRKLYHVVKYTGGS
AELLFFVEGLCFPDEGFSGLVSIHVSLLEYMAQDIPLTPIFTDTVIFRI
APWIMTPNILPPVSVFVCCMKDNYLFLKEVKNLVEKTNCELKVCFQYLN
RGDRWIQDEIEFGYIEAPHKGFPVVLDSPRDGNLKDFPVKELLGPDFGY
VTREPLFESVTSLDSFGNLEVSPPVTVNGKTYPLGRILIGSSFPLSGGR
RMTKVVRDFLKAQQVQAPVELYSDWLTVGHVDEFMSFVPIPGTKKFLLL
MASTSACYKLFREKQKDGHGEAIMFKGLGGMSSKRITINKILSNESLVQ
ENLYFQRCLDWNRDILKKELGLTEQDIIDLPALFKMDEDHRARAFFPNM
VNMIVLDKDLGIPKPFGPQVEEECCLEMHVRGLLEPLGLECTFIDDISA
YHKFLGEVHCGTNVRRKPFTFKWWHMVP

As used herein, the term “peptidyl arginine deiminase 3” or “PAD3,” also known as PADI3, PDI3 and UHS1, refers to a member of the PAD family of enzymes. PAD3 is detected in the epidermis where it plays a role in terminal differentiation and in hair follicles where it modulates structural proteins including filaggrin and trichoyalin. See Chavanas et al., J Dermatol Sci., 44:63-72 (2006); Kanno et al., J. Invest Dermatol. 115(5):813-23 (2000); Nachat et al., J Investig Dermatol., 125:34-41 (2005). Approximately 738 coding SNPs have been identified for PAD3 and at least 109 known orthologs (see, for example, NCBI Gene ID: 51702). The term “PAD3” includes all of such PAD3 variants and PAD3 orthologs. An exemplary human PAD3 (hPAD3) nucleotide sequence can be found in GenBank under GenBank Accession number NM_016233 (SEQ ID NO:5) and encodes an exemplary human PAD3 having the amino acid sequence found under found under GenBank Accession number NP_057317(SEQ ID NO:6). The GenBank Accession numbers and GenBank GI numbers of this PAD3 and other exemplary PAD3_enzymes can be found below in Table 2. All of such PAD3 polypeptides and variants thereof are included within the meaning of the term “PAD3” as it is used herein.

In some embodiments, a PAD3, or antigenic fragment thereof, includes the amino acid in SEQ ID NO:6 of a mature human PAD3 (hPAD3; amino acids 25-664 of NCBI Accession Number NP_057317; GI: 122939161), or naturally occurring variants thereof:

SEQ ID NO: 6
MSLQRIVRVSLEHPTSAVCVAGVETLVDIYGSVPEGTEMFEVYGTPGVD
IYISPNMERGRERADTRRWRFDATLEIIVVMNSPSNDLNDSHVQISYHS
SHEPLPLAYAVLYLTCVDISLDCDLNCEGRQDRNFVDKRQWVWGPSGYG
GILLVNCDRDDPSCDVQDNCDQHVHCLQDLEDMSVMVLRTQGPAALFDD
HKLVLHTSSYDAKRAQVFHICGPEDVCEAYRHVLGQDKVSYEVPRLHGD
EERFFVEGLSFPDAGFTGLISFHVTLLDDSNEDFSASPIFTDTVVFRVA
PWIMTPSTLPPLEVYVCRVRNNTCFVDAVAELARKAGCKLTICPQAENR
NDRWIQDEMELGYVQAPHKTLPVVFDSPRNGELQDFPYKRILGPDFGYV
TREPRDRSVSGLDSFGNLEVSPPVVANGKEYPLGRILIGGNLPGSSGRR
VTQVVRDFLHAQKVQPPVELFVDWLAVGHVDEFLSFVPAPDGKGFRMLL
ASPGACFKLFQEKQKCGHGRALLFQGVVDDEQVKTISINQVLSNKDLIN
YNKFVQSCIDWNREVLKRELGLAECDIIDIPQLFKTERKKATAFFPDLV
NMLVLGKHLGIPKPFGPIINGCCCLEEKVRSLLEPLGLHCTFIDDFTPY
HMLHGEVHCGTNVCRKPFSFKWWNMVP

As used herein, the term “peptidyl arginine deiminase 4” or “PAD4,” also known as PAD, PADI4, PDI4, PADV, PDI5 and PADI5, refers to a member of the PAD family of enzymes. PAD4 can be detected in white blood cells including granulocytes and monocytes under normal physiological conditions (Vossenaar et al., Annals of the Rheumatic Diseases, 63:373-81 (2004); Asaga et al., J. Leukocyte Biology 70:46-51 (2001)) and is generally localized in the nucleus (Nakashima et al., JBC 277:49562-68 (2002)). Approximately 737 coding SNPs have been identified for PAD4 and at least 108 known orthologs (see, for example, NCBI Gene ID: 23569). The term “PAD4” includes all of such PAD4 variants and PAD4 orthologs. An exemplary human PAD4 (hPAD4) nucleotide sequence can be found in GenBank under GenBank Accession number NM_012387.2 (SEQ ID NO:61) and encodes an exemplary human PAD4 having the amino acid sequence found under found under GenBank Accession number NP_036519.2 (SEQ ID NO:62). The GenBank Accession numbers and GenBank GI numbers of this PAD4 and other exemplary PAD4 enzymes can be found below in Table 3. All of such PAD4 polypeptides and variants thereof are included within the meaning of the term “PAD4” as it is used herein.

In some embodiments, a PAD4, or antigenic fragment thereof, includes the amino acid in SEQ ID NO:62, of a mature human PAD4 (hPAD4; amino acids 25-663 of NCBI Accession Number NP_036519; GI: 216548487), or naturally occurring variants thereof:

SEQ ID NO: 62
MAQGTLIRVTPEQPTHAVCVLGTLTQLDICSSAPEDCTSFSINASPGVV
VDIAHGPPAKKKSTGSSTWPLDPGVEVTLTMKVASGSTGDQKVQISYYG
PKTPPVKALLYLTGVEISLCADITRTGKVKPTRAVKDQRTWTWGPCGQG
AILLVNCDRDNLESSAMDCEDDEVLDSEDLQDMSLMTLSTKTPKDFFTN
HTLVLHVARSEMDKVRVFQATRGKLSSKCSVVLGPKWPSHYLMVPGGKH
NMDFYVEALAFPDTDFPGLITLTISLLDTSNLELPEAVVFQDSVVFRVA
PWIMTPNTQPPQEVYACSIFENEDFLKSVTTLAMKAKCKLTICPEEENM
DDQWMQDEMEIGYIQAPHKTLPVVFDSPRNRGLKEFPIKRVMGPDFGYV
TRGPQTGGISGLDSFGNLEVSPPVTVRGKEYPLGRILFGDSCYPSNDSR
QMHQALQDFLSAQQVQAPVKLYSDWLSVGHVDEFLSFVPAPDRKGFRLL
LASPRSCYKLFQEQQNEGHGEALLFEGIKKKKQQKIKNILSNKTLREHN
SFVERCIDWNRELLKRELGLAESDIIDIPQLFKLKEFSKAEAFFPNMVN
MLVLGKHLGIPKPFGPVINGRCCLEEKVCSLLEPLGLQCTFINDFFTYH
IRHGEVHCGTNVRRKPFSFKWWNMVP

Tables 1, 2 and 3 contain two sequence identifiers, the GI number and the GenBank accession number. The GI number and GenBank accession number run in parallel as unique identifiers to access the referenced sequence in publicly available databases. Table 1 includes GI numbers and GenBank Accession numbers for PAD2, Table 2 includes GI numbers and GenBank Accession numbers for PAD3 and Table 3 includes GI numbers and GenBank accession numbers for PAD4.

The sequence identifiers in Tables 1, 2 and 3 are provided for wild-type PAD2, 3 and 4, respectively. It should be understood that wild-type nucleic acid and amino acid sequences herein refer to those nucleic acid and amino acid sequences prevalent among a population and serve as a reference for their respective variants. As an example, SEQ ID NO:61 in Table 3 (GI number: 1519314340 and Accession number: NM_012387) identifies the wild-type nucleic acid sequence for hPAD4 while SEQ ID NO:62 (GI number: 216548487 and Accession number: NP_036519) identifies the wild-type amino acid sequence for hPAD4.

The sequence identifiers in Tables 1, 2 and 3 also are provided for variants of PAD2, 3 and 4 respectively. It should be understood that a variant refers to a nucleic acid sequence that is similar but different from the wild-type nucleic acid sequence.

A variant in any of the Tables below can include a nucleic acid substitution that can be the result of, for example, alternative splicing (e.g. splice variant). As an example, SEQ ID NO: 69 in Table 3 (GI number: 767903519 and Accession number: XM_011541150.1: c.23G>A) is a hPAD4 nucleic acid splice variant of SEQ ID NO:61.

A variant in any of the Tables below can also include, for example, a nucleic acid substitution (e.g. SNP). As an example, SEQ ID NO:63 in Table 3 (GI number: 216548486 and Accession number: NM_012387.2:c.23G>A) is a hPAD4 nucleic acid variant of SEQ ID NO:61 and includes a single nucleic acid substitution at nucleic acid position 23, resulting in the substitution of G (guanosine) for A (adenine).

It should be understood that a variant also refers to an amino acid sequence that is similar but different to the wild-type amino acid sequence.

A variant in any of the Tables below can further include amino acid substitutions that can be the result of, for example, alternative splicing (e.g. splice variant). As an example, SEQ ID NO: 70 in Table 3 (GI number: 767903520 and Accession number: XP_011539452.1: p.Arg8His), which is encoded by SEQ ID NO:69 described above, is a hPAD4 that includes an amino acid substitution at position 8, resulting in a substitution of Arg (arginine) for His (histidine).

A variant in any of the Tables below can include, for example, amino acid substitutions that can be the result of genetic inheritance (e.g. SNP). As an example, SEQ ID NO: 64 in Table 3 (GI number: 216548487 and Accession number: NP_036519.2: p.Arg8His), which is encoded by SEQ ID NO:63 described above, is a hPAD4 that includes an amino acid substitution at position 8, resulting in a substitution of Arg (arginine) for His (histidine).

TABLE 1

| | | | GenBank |
|---|---|---|---|
| | SEQ | GI | Accession |
| Molecule Type | ID NO | Number | Number |
| Homo sapiens peptidyl arginine | 1 | 1519245591 | NM_{—} |
| deiminase 2 (PADI2), mRNA | | | 007365 |
| protein-arginine deiminase | 2 | 122939159 | NP_{—} |
| type-2 [Homo sapiens] | | | 031391 |
| PREDICTED: Homo sapiens | 3 | 1370451734 | XM_{—} |
| peptidyl arginine deiminase 2 | | | 017000148 |
| (PADI2), transcript variant X2, | | | |
| mRNA | | | |
| protein-arginine deiminase type- | 4 | 1034554998 | XP_{—} |
| 2 isoform X1 [Homo sapiens] | | | 016855637 |

TABLE 2

| | | | GenBank | | |
|---|---|---|---|---|---|
| | SEQ ID | GI | Accession | Amino Acid | SO |
| Molecule Type | NO | Number | Number | [Codon] | Term |
| Homo sapiens peptidyl | 5 | 122939160 | NM_016233 | N/A | N/A |
| arginine deiminase 3 | | | | | |
| (PADI3), mRNA | | | | | |
| protein-arginine deiminase | 6 | 122939161 | NP_057317 | N/A | N/A |
| type-3 [Homo sapiens] | | | | | |
| PADI3 transcript | 7 | 122939160 | NM_016233.2: | I [ATC] > V | Coding |
| | | | c.154A > G | [GTC] | Sequence |
| | | | | | Variant |
| protein-arginine deiminase | 8 | 122939161 | NP_057317.2: | I (Ile) > V | Missense |
| type-3 | | | p.Ile52Val | (Val) | Variant |
| PADI3 transcript | 9 | 122939160 | NM_016233.2: | L [CTC] > H | Coding |
| | | | c.335T > A | [CAC] | Sequence |
| | | | | | Variant |
| protein-arginine deiminase | 10 | 122939161 | NP_057317.2: | L (Leu) > H | Missense |
| type-3 | | | p.Leu112His | (His) | Variant |
| PADI3 transcript | 11 | 122939160 | NM_016233.2: | V [GTG] > | Coding |
| | | | c.511G > A | M [ATG] | Sequence |
| | | | | | Variant |
| protein-arginine deiminase | 12 | 122939161 | NP_057317.2: | V (Val) > M | Missense |
| type-3 | | | p.Val171Met | (Met) | Variant |
| PADI3 transcript | 13 | 122939160 | NM_016233.2: | A [GCA] > | Coding |
| | | | c.881C > T | V [GTA] | Sequence |
| | | | | | Variant |
| protein-arginine deiminase | 14 | 122939161 | NP_057317.2: | A (Ala) > V | Missense |
| type-3 | | | p.Ala294Val | (Val) | Variant |
| PADI3 transcript | 15 | 122939160 | NM_016233.2: | A [GCC] > T | Coding |
| | | | c.1744G > A | [ACC] | Sequence |
| | | | | | Variant |
| protein-arginine deiminase | 16 | 122939161 | NP_057317.2: | A (Ala) > T | Missense |
| type-3 | | | p.Ala582Thr | (Thr) | Variant |
| PADI3 transcript | 17 | 122939160 | NM_016233.2: | P [CCC] > T | Coding |
| | | | c.1813C > A | [ACC] | Sequence |
| | | | | | Variant |
| protein-arginine deiminase | 18 | 122939161 | NP_057317.2: | P (Pro) > T | Missense |
| type-3 | | | p.Pr0605Thr | (Thr) | Variant |
| PADI3 transcript | 19 | 122939160 | NM_016233.2: | R [CGG] > Q | Coding |
| | | | c.1853G > A | [CAG] | Sequence |
| | | | | | Variant |
| protein-arginine deiminase | 20 | 122939161 | NP_057317.2: | R (Arg) > Q | Missense |
| type-3 | | | p.Arg618Gln | (Gln) | Variant |
| Predicted: Homo sapiens | 21 | 1034559140 | XM_011541571 | N/A | N/A |
| peptidyl arginine deiminase 3 | | | | | |
| (PADI3), transcript variant | | | | | |
| X1, mRNA | | | | | |
| protein-arginine deiminase | 22 | 767904616 | XP_011539873 | N/A | N/A |
| type-3 isoform X1 [Homo | | | | | |
| sapiens] | | | | | |
| Predicted: PADI3 transcript | 23 | 1034559140 | XM_011541571.2: | I [ATC] > V | Coding |
| variant X1 | | | c.40A > G | [GTC] I (Ile) > | Sequence |
| | | | | V (Val) | Variant |
| protein-arginine deiminase | 24 | 767904616 | XP_011539873.1: | | Missense |
| type-3 isoform X1 | | | p.Ile14Val | | Variant |
| Predicted: PADI3 transcript | 25 | 1034559140 | XM_011541571.2: | L [CTC] > H | Coding |
| variant X1 | | | c.221T > A | [CAC] | Sequence |
| | | | | | Variant |
| protein-arginine deiminase | 26 | 767904616 | XP_011539873.1: | L (Leu) > H | Missense |
| type-3 isoform X1 | | | p.Leu74His | (His) | Variant |
| Predicted: PADI3 transcript | 27 | 1034559140 | XM_011541571.2: | V [GTG] > | Coding |
| variant X1 | | | c.397G > A | M [ATG] | Sequence |
| | | | | | Variant |
| protein-arginine deiminase | 28 | 767904616 | XP_011539873.1: | V (Val) > M | Missense |
| type-3 isoform X1 | | | p.Val133Met | (Met) | Variant |
| PADI3 transcript variant X1 | 29 | 1034559140 | XM_011541571.2: | A [GCA] > | Coding |
| | | | c.767C > T | V [GTA] | Sequence |
| | | | | | Variant |
| protein-arginine deiminase | 30 | 767904616 | XP_011539873.1: | A (Ala) > V | Missense |
| type-3 isoform X1 | | | p.Ala256Val | (Val) | Variant |
| PADI3 transcript variant X1 | 31 | 1034559140 | XM_011541571.2: | A [GCC] > T | Coding |
| | | | c.1630G > A | [ACC] | Sequence |
| | | | | | Variant |
| protein-arginine deiminase | 32 | 767904616 | XP_011539873.1: | A (Ala) > T | Missense |
| type-3 isoform X1 | | | p.Ala544Thr | (Thr) | Variant |
| PADI3 transcript variant X1 | 33 | 1034559140 | XM_011541571.2: | P [CCC] > T | Coding |
| | | | c.1699C > A | [ACC] | Sequence |
| | | | | | Variant |
| protein-arginine deiminase | 34 | 767904616 | XP_011539873.1: | P (Pro) > T | Missense |
| type-3 isoform X1 | | | p.Pro567Thr | (Thr) | Variant |
| PADI3 transcript variant X1 | 35 | 1034559140 | XM_011541571.2: | R [CGG] > Q | Coding |
| | | | c.1739G > A | [CAG] | Sequence |
| | | | | | Variant |
| protein-arginine deiminase | 36 | 767904616 | XP_011539873.1: | R (Arg) > Q | Missense |
| type-3 isoform X1 | | | p.Arg580Gln | (Gln) | Variant |
| Homo sapiens peptidyl | 37 | 1034559141 | XM_017001463 | N/A | N/A |
| arginine deiminase 3 | | | | | |
| (PADI3), transcript variant | | | | | |
| X2, mRNA | | | | | |
| protein-arginine deiminase | 38 | 1034559142 | XP_016856952 | N/A | N/A |
| type-3 isoform X2 [Homo | | | | | |
| sapiens] | | | | | |
| PADI3 transcript variant X2 | 39 | 1034559141 | XM_017001463.1: c | N/A | Genic |
| | | | | | Upstream |
| | | | | | Transcript |
| | | | | | Variant |
| PADI3 transcript variant X2 | 40 | 1034559141 | XM_017001463.1: c | N/A | 5 Prime UTR |
| | | | | | Variant |
| PADI3 transcript variant X2 | 41 | 1034559141 | XM_017001463.1: | A [GCA] > | Coding |
| | | | c.344C > T | V [GTA] | Sequence |
| | | | | | Variant |
| protein-arginine deiminase | 42 | 1034559142 | XP_016856952.1: | A (Ala) > V | Missense |
| type-3 isoform X2 | | | p.Ala115Val | (Val) | Variant |
| PADI3 transcript variant X2 | 43 | 1034559141 | XM_017001463.1: | A [GCC] > T | Coding |
| | | | c.1207G > A | [ACC] | Sequence |
| | | | | | Variant |
| protein-arginine deiminase | 44 | 1034559142 | XP_016856952.1: | A (Ala) > T | Missense |
| type-3 isoform X2 | | | p.Ala403Thr | (Thr) | Variant |
| PADI3 transcript variant X2 | 45 | 1034559141 | XM_017001463.1: | P [CCC] > T | Coding |
| | | | c.1276C > A | [ACC] | Sequence |
| | | | | | Variant |
| protein-arginine deiminase | 46 | 1034559142 | XP_016856952.1: | P (Pro) > T | Missense |
| type-3 isoform X2 | | | p.Pro426Thr | (Thr) | Variant |
| PADI3 transcript variant X2 | 47 | 1034559141 | XM_017001463.1: | R [CGG] > Q | Coding |
| | | | c.1316G > A | [CAG] | Sequence |
| | | | | | Variant |
| protein-arginine deiminase | 48 | 1034559142 | XP_016856952.1: | R (Arg) > Q | Missense |
| type-3 isoform X2 | | | p.Arg439Gln | (Gln) | Variant |
| Homo sapiens peptidyl | 49 | 1034559143 | XM_011541572 | N/A | N/A |
| arginine deiminase 3 | | | | | |
| (PADI3), transcript variant | | | | | |
| X3, mRNA | | | | | |
| protein-arginine deiminase | 50 | 767904618 | XP_011539874 | N/A | N/A |
| type-3 isoform X3 [Homo | | | | | |
| sapiens] | | | | | |
| PADI3 transcript variant X3 | 51 | 1034559143 | XM_011541572.2: | I [ATC] > V | Coding |
| | | | c.154A > G | [GTC] | Sequence |
| | | | | | Variant |
| protein-arginine deiminase | 52 | 767904618 | XP_011539874.1: | I (Ile) > V | Missense |
| type-3 isoform X3 | | | p.Ile52Val | (Val) | Variant |
| PADI3 transcript variant X3 | 53 | 1034559143 | XM_011541572.2: | L [CTC] > H | Coding |
| | | | c.335T > A | [CAC] | Sequence |
| | | | | | Variant |
| protein-arginine deiminase | 54 | 767904618 | XP_011539874.1: | L (Leu) > H | Missense |
| type-3 isoform X3 | | | p.Leu112His | (His) | Variant |
| PADI3 transcript variant X3 | 55 | 1034559143 | XM_011541572.2: | V [GTG] > | Coding |
| | | | c.511G > A | M [ATG] | Sequence |
| | | | | | Variant |
| protein-arginine deiminase | 56 | 767904618 | XP_011539874.1: | V (Val) > M | Missense |
| type-3 isoform X3 | | | p.Val171Met | (Met) | Variant |
| PADI3 transcript variant X3 | 57 | 1034559143 | XM_011541572.2: | A [GCA] > | Coding |
| | | | c.881C > T | V [GTA] | Sequence |
| | | | | | Variant |
| protein-arginine deiminase | 58 | 767904618 | XP_011539874.1: | A (Ala) > V | Missense |
| type-3 isoform X3 | | | p.Ala294Val | (Val) | Variant |
| PADI3 transcript variant X3 | 59 | 1034559143 | XM_011541572.2: c. | N/A | Genic |
| | | | | | Downstream |
| | | | | | Transcript |
| | | | | | Variant |

TABLE 3

| | | | GenBank | Amino | |
|---|---|---|---|---|---|
| | SEQ ID | GI | Accession | Acid | |
| Molecule Type | NO | Number | Number | [Codon] | SO Term |
| Homo sapiens peptidyl | 61 | 1519314340 | NM_012387 | N/A | N/A |
| arginine deiminase 4 | | | | | |
| (PADI4), mRNA | | | | | |
| protein-arginine deiminase | 62 | 216548487 | NP_036519 | N/A | N/A |
| type-4 [Homo sapiens] | | | | | |
| PADI4 transcript | 63 | 216548486 | NM_012387.2: | R [CGT] > | Coding Sequence |
| | | | c.23G > A | H [CAT] | Variant |
| protein-arginine deiminase | 64 | 216548487 | NP_036519.2: | R (Arg) > H | Missense Variant |
| type-4 | | | p.Arg8His | (His) | |
| PADI4 transcript | 65 | 216548486 | NM_012387.2: | R [CGT] > | Coding Sequence |
| | | | c.23G > T | L [CTT] | Variant |
| protein-arginine deiminase | 66 | 216548487 | NP_036519.2: | R (Arg) > L | Missense Variant |
| type-4 | | | p.Arg8Leu | (Leu) | |
| PADI4 transcript variant X3 | 67 | 767903523 | XM_011541152.1: c. | N/A | Genic Upstream |
| | | | | | Transcript Variant |
| PADI4 transcript variant X8 | 68 | 767903533 | XM_011541157.1: c. | N/A | Genic Upstream |
| | | | | | Transcript Variant |
| PADI4 transcript variant X1 | 69 | 767903519 | XM_011541150.1: | R [CGT] > | Coding Sequence |
| | | | c.23G > A | H [CAT] | Variant |
| protein-arginine deiminase | 70 | 767903520 | XP_011539452.1: | R (Arg) > H | Missense Variant |
| type-4 isoform X1 | | | p.Arg8His | (His) | |
| PADI4 transcript variant X1 | 71 | 767903519 | XM_011541150.1: | R [CGT] > | Coding Sequence |
| | | | c.23G > T | L [CTT] | Variant |
| protein-arginine deiminase | 72 | 767903520 | XP_011539452.1: | R (Arg) > L | Missense Variant |
| type-4 isoform X1 | | | p.Arg8Leu | (Leu) | |
| PADI4 transcript variant X2 | 73 | 767903521 | XM_011541151.1: | R [CGT] > | Coding Sequence |
| | | | c.23G > A | H [CAT] | Variant |
| protein-arginine deiminase | 74 | 767903522 | XP_011539453.1: | R (Arg) > H | Missense Variant |
| type-4 isoform X2 | | | p.Arg8His | (His) | |
| PADI4 transcript variant X2 | 75 | 767903521 | XM_011541151.1: | R [CGT] > | Coding Sequence |
| | | | c.23G > T | L [CTT] | Variant |
| protein-arginine deiminase | 76 | 767903522 | XP_011539453.1: | R (Arg) > L | Missense Variant |
| type-4 isoform X2 | | | p.Arg8Leu | (Leu) | |
| PADI4 transcript variant X4 | 77 | 767903525 | XM_011541153.1: | R [CGT] > | Coding Sequence |
| | | | c.23G > A | H [CAT] | Variant |
| protein-arginine deiminase | 78 | 767903526 | XP_011539455.1: | R (Arg) > H | Missense Variant |
| type-4 isoform X4 | | | p.Arg8His | (His) | |
| PADI4 transcript variant X4 | 79 | 767903525 | XM_011541153.1: | R [CGT] > | Coding Sequence |
| | | | c.23G > T | L [CTT] | Variant |
| protein-arginine deiminase | 80 | 767903526 | XP_011539455.1: | R (Arg) > L | Missense Variant |
| type-4 isoform X4 | | | p.Arg8Leu | (Leu) | |
| PADI4 transcript variant X6 | 81 | 767903529 | XM_011541155.1: | R [CGT] > | Coding Sequence |
| | | | c.23G > A | H [CAT] | Variant |
| protein-arginine deiminase | 82 | 767903530 | XP_011539457.1: | R (Arg) > H | Missense Variant |
| type-4 isoform X5 | | | p.Arg8His | (His) | |
| PADI4 transcript variant X6 | 83 | 767903529 | XM_011541155.1: | R [CGT] > | Coding Sequence |
| | | | c.23G > T | L [CTT] | Variant |
| protein-arginine deiminase | 84 | 767903530 | XP_011539457.1: | R (Arg) > L | Missense Variant |
| type-4 isoform X5 | | | p.Arg8Leu | (Leu) | |
| PADI4 transcript variant X7 | 85 | 767903531 | XM_011541156.1: | R [CGT] > | Coding Sequence |
| | | | c.23G > A | H [CAT] | Variant |
| protein-arginine deiminase | 86 | 767903532 | XP_011539458.1: | R (Arg) > H | Missense Variant |
| type-4 isoform X6 | | | p.Arg8His | (His) | |
| PADI4 transcript variant X7 | 87 | 767903531 | XM_011541156.1: | R [CGT] > | Coding Sequence |
| | | | c.23G > T | L [CTT] | Variant |
| protein-arginine deiminase | 88 | 767903532 | XP_011539458.1: | R (Arg) > L | Missense Variant |
| type-4 isoform X6 | | | p.Arg8Leu | (Leu) | |
| PADI4 transcript variant X5 | 89 | 1034557308 | XM_011541154.2: | R [CGT] > | Coding Sequence |
| | | | c.23G > A | H [CAT] | Variant |
| protein-arginine deiminase | 90 | 767903528 | XP_011539456.1: | R (Arg) > H | Missense Variant |
| type-4 isoform X4 | | | p.Arg8His | (His) | |
| PADI4 transcript variant X5 | 91 | 1034557308 | XM_011541154.2: | R [CGT] > | Coding Sequence |
| | | | c.23G > T | L [CTT] | Variant |
| protein-arginine deiminase | 92 | 767903528 | XP_011539456.1: | R (Arg) > L | Missense Variant |
| type-4 isoform X4 | | | p.Arg8Leu | (Leu) | |

It should be noted that “polypeptide” includes a short oligopeptide having between 2 and 30 amino acids (e.g., 2, 3, 4, 5, 6, 7, 8, 9, 10, 12, 14, 16, 18, 20, 25 or 30 amino acids) as well as longer amino acid chains, e.g., more than 30 amino acids, more than 50 amino acids, more than 100 amino acids, more than 150 amino acids, more than 200 amino acids, more than 300 amino acids, more than 400 amino acids, more than 500 amino acids, or more than 600 amino acids. In some embodiments, the PAD can be a full-length, wild-type polypeptide. PAD polypeptides can include unnatural amino acids not encoded by the natural genetic code.

In some embodiments, the purified polypeptide includes a PAD antigenic fragment. In some embodiments, a PAD antigenic fragment includes more than 3, more than 5, more than 10, more than 15, more than 20, more than 25, more than 50, more than 75, more than 100, more than 125, more than 150, more than 200, more than 250, more than 300, more than 350, more than 400, more than 500, or more than 600 consecutive amino acids of a full-length PAD polypeptide. In some embodiments, a PAD antigenic fragment includes less than 100%, less than 95%, less than 90%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% of consecutive amino acids of full-length PAD. In some embodiments, a PAD antigenic fragment is a PAD peptide fragment.

In some embodiments, a PAD or antigenic fragment thereof can be a mammalian PAD. In some embodiments, a PAD or antigenic fragment thereof can be human. In some embodiments, a PAD or antigenic fragment thereof can be a PAD or antigenic fragment thereof of one of the organisms of the present disclosure. In some embodiments, a PAD or antigenic fragment thereof can include any of the variants or single nucleotide polymorphisms in Tables 1-3.

A PAD or antigenic fragment thereof can be obtained using various methods well known in the art. For example, a PAD or antigenic fragment thereof can be isolated from a natural source, produced by chemical synthesis or produced by recombinant protein expression.

Exemplary methods for expressing and purifying recombinant polypeptides, for purifying polypeptides from cells, tissues or bodily fluids, and for chemically synthesizing polypeptides are well known in the art and can be found described in Scopes R. K., Protein Purification—Principles and Practice, Springer Advanced Texts in Chemistry, 3rd Edition (1994); Simpson R. J. et al., Basic Methods in Protein Purification and Analysis: A Laboratory Manual, Cold Spring Harbor Laboratory Press, 1st Edition (2008); Green M. R. and Sambrook J., Molecular Cloning: A Laboratory Manual, Cold Spring Harbor Laboratory Press, 4th Edition (2012); Jensen K. J. et al., Peptide Synthesis and Applications (Methods in Molecular Biology), Humana Press, 2nd Edition (2013).

Polypeptides purified or isolated from a natural source refers to the isolation and purification of a polypeptide from a source where it is naturally expressed. In some embodiments, a natural source of a PAD can be from a cell, tissue or bodily fluid of an organism. In some embodiments, the cells, tissues or bodily fluids can include, for example, whole blood, serum, plasma, synovial fluid or sputum from an organism of the present disclosure. A PAD or antigenic fragment thereof can similarly be isolated from any biological sample described and provided herein.

It should be noted that the terms “purified” or “isolated” refer to a polypeptide that is isolated, partially or completely, from a complex mixture of components, as found in nature. Thus, in some embodiments, a PAD of the present disclosure can be partially purified or substantially purified. Partial purification results in isolation from one or more components as found in nature. Substantial purification results in isolation from all components as found in nature. Partial purification, as disclosed herein, can be achieved by the methods and compositions provided herein. In some embodiments, a partially purified PAD can be performed with a capture probe. In some embodiments, the capture probe is a polypeptide or functional fragment thereof specific to PAD. In some embodiments, the capture probe is an anti-PAD antibody. Substantial purification, as exemplified herein, can be achieved by methods known in the art. In some embodiments, a PAD is purified substantially by a process of extraction, precipitation and solubilization.

Recombinant polypeptides can be expressed in and purified from bacterial cells (e.g., E. coli), yeast cells (e.g., S. cerevisiae), insect cells (e.g., Sf9), in mammalian cells (e.g., CHO) and others. Recombinant polypeptides can be expressed and purified as fusion proteins including tags for protein detection or affinity purification tags (e.g., His-tag, GST-tag, Myc-tag), including cleavable tags (e.g., tags including a TEV-cleavage site). In some embodiments, the PAD provided herein can be purified from a cell, tissue or bodily fluid obtained from an organism. Tissues or bodily fluids can include any tissue or bodily fluids obtained from the organism. In some embodiments, the tissues or bodily fluids can include blood, serum, plasma, synovial fluid, urine or milk (e.g., from goats, cows, sheep). One skilled in the art will recognize that methods for the purification of polypeptides from cells, tissues or bodily fluids are well known in the art.

In some embodiments, a PAD or antigenic fragment thereof is chemically synthesized using, for example, methods described in Jensen, K. J. (supra).

In some embodiments, a PAD antigenic fragment can be produced by enzymatically digesting full-length PAD. The full-length PAD can be obtained by, for example, any of the exemplary methods described above. The enzymatic digest can be carried out with, for example, a protease or peptidase. In some embodiments, the protease or peptidase can be an exoprotease or an exopeptidase. In some embodiments, the protease or peptidase can be an endoprotease or endopeptidase. In some embodiments, the protease or peptidase can include a serine protease, threonine protease, cysteine protease, aspartate protease, glutamic acid protease, or metalloprotease. In some embodiments, the protease or peptidase can include trypsin, chymotrypsin, pepsin, papain and any cathepsin including cathepsin B, L, D, K, or G.

In some embodiments, a PAD or antigenic fragment thereof can be a native PAD. In some embodiments, the PAD or antigenic fragment thereof can be a denatured or unfolded PAD. In some embodiments, the PAD or antigenic fragment thereof can include unnatural amino acids. In some embodiments, the unnatural amino acids can be methylated at the-amino-group to produce polypeptides with methylated backbones. In some embodiments, the unnatural amino acids can be R-amino acids. In some embodiments, the unnatural amino acids can include dyes (e.g., fluorescent dyes) or affinity tags. In some embodiments, the PAD or antigenic fragment thereof can include chemical modifications. Chemical modifications can include, e.g., chemical modifications with biotin, fluorescent dyes. A skilled artisan will recognize that methods for introducing unnatural amino acids into polypeptides and for chemically modifying polypeptides are well known in the art.

In some embodiments, an isolated, chemically synthesized or recombinant PAD or antigenic fragment thereof can be a plurality of PADs. It should be noted that the term “plurality” refers to a population of two or more members, such as polypeptide members or other referenced molecules. In some embodiments, the two or more members of a plurality of members can be the same members. For example, a plurality of polypeptides can include two or more polypeptide members having the same amino acid sequence. By way of exemplification, a plurality of members having the same amino acid sequence can include two or more members of any one of PAD exemplified in Table 1. In some embodiments, the two or more members of a plurality of members can be different members. For example, a plurality of polypeptides can include two or more polypeptide members having different amino acid sequences. By way of exemplification, a plurality of members having different amino acid sequences can include at least one member of two or more PADs exemplified in Table 1. A plurality includes 2, 3, 4, 5, 6, 7, 8, 9, 10, 20, 30, 40, 50, 60, 70, 80, 90 or a 100 or more different members. A plurality can also include 200, 300, 400, 500, 1000, 5000, 10000, 50000, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶ or 1×10⁷ or more different members. A plurality includes, for example, all integer numbers in between the above exemplary plurality numbers. In some embodiments, a PAD can be a plurality of PADs from the organisms of the present disclosure.

As provided herein, RA, RA severity and joint erosion can be determined in subjects of the present disclosure by the detection of anti-PAD2, anti-PAD3 or anti-PAD4 IgA. Detection of any of the anti-PAD IgA described herein can be performed through the use of, for example, an antibody specific to IgA or other binding molecule specific to IgA. An IgA binding molecule in the art can be used. An antibody or other binding molecule specific to any of the anti-PAD IgA described herein can also be employed.

As used herein, the term “antibody” is used interchangeably with immunoglobulin (Ig) and refers to a polypeptide product of B-cells that is able to bind to a specific molecular antigen and is composed of two heavy chains and two light chains. Each amino-terminal portion of each chain includes a variable region that confers binding specificity. See Borrebaeck (ed.) (1995) Antibody Engineering, Second Edition, Oxford University Press.; Kuby (1997) Immunology, Third Edition, W.H. Freeman and Company, New York. The term includes autoantibodies and antibodies used as detection probes in the disclosed methods. The antibody can exhibit specific binding affinity where it binds to a single molecular species or pan-specific binding where it binds selectively to more than one related molecular species. In the context of the present disclosure, the specific molecular antigen that can be bound by an antibody of the disclosure includes, for example, IgA, PAD (e.g., PAD2, PAD3 and/or PAD4), PAD: anti-PAD IgA complex (e.g., anti-PAD 2, 3 and/or 4 IgA complexes), and/or anti-PAD IgA (e.g., anti-PAD2 IgA, anti-PAD3 IgA and/or anti-PAD4 IgA). An antibody of the present disclosure can be derived from any mammalian organism, including mouse, rabbit, goat, chicken, donkey and the like. Furthermore, a primary or secondary antibody can be monoclonal, polyclonal, chimeric or humanized. The antibodies provided herein can be used in the methods and compositions of the disclosure.

As used herein, the term “functional fragment” when used in reference to an antibody is intended to refer to a portion of the antibody including heavy or light chain polypeptides that retain some or all of the binding activity as the antibody from which the fragment was derived. Such functional fragments can include, for example, an Fd, Fv, Fab, F (ab′), F(ab)2, F(ab′)2, single chain Fv (scFv), diabody, triabody, tetrabody and minibody. Such antibody binding fragments can be found described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics, 22:189-224 (1993); Plückthun and Skerra, Meth. Enzymol., 178:497-515 (1989) and in Day, E.D., Advanced Immunochemistry, Second Ed., Wiley-Liss, Inc., New York, NY (1990). The antibody functional fragments provided herein can be used in the methods and compositions of the disclosure. Ligands are provided herein and include any molecule having specific binding to a target. Exemplary ligands include a polypeptide, IgA binding molecules including, for example, IgA binding proteins, an affibody, an aptamer, a small molecule and the like. Specific examples of polypeptide ligands include receptors, chimeric receptors and polypeptides identified from screening of random or combinatorial libraries. Exemplary polypeptide ligands of the present disclosure include PAD (e.g., PAD2, PAD3 and/or PAD4) or an antigenic fragment thereof or an IgA binding polypeptide. An exemplary IgA binding polypeptide includes KW-0388. Other exemplary ligands that bind to IgA can be found described in Rönnmark et al., “Human immunoglobulin A (IgA)-specific ligands from combinatorial engineering of protein A,” Eur. J. Biochem. 269:2647-55 (2002) and Kruljec et al., “Alternative Affinity Ligands for Immunoglobulins,” Bioconjugate Chem. 28:2009-30 (2017). A ligand of the present disclosure can be obtained or synthesized by methods described herein or known in the art, including for example, chemically synthesized, purified from a natural source or recombinantly made. Thus, a ligand detection probe described herein can be mammalian, including mouse, rabbit, goat, chicken, donkey and the like. All of such ligands provided herein can be used in the methods and compositions of the disclosure.

As used herein, the term “detection probe” refers to a binding agent capable of specific binding to a target. Such binding agents include, for example, antibodies and ligands. Antibodies include full length antibodies as well as functional fragments such as those exemplified above. Ligands include full length polypeptides such as those exemplified above and functional binding fragments thereof. Ligands also include the non-polypeptide ligands exemplified above. When referring to specific binding to a target, a detection probe of the disclosure can bind the target directly or it can be made specific to the target by indirect means. For example, a detection probe that binds directly to anti-PAD IgA includes PAD. A direct binder also includes, for example, an antibody or other ligand that specifically recognizes a PAD: anti-PAD IgA complex as well as an antibody or other ligand that specifically binds to anti-PAD IgA. A detection probe of the disclosure that can be made specific to anti-PAD IgA by indirect means includes, for example, anti-IgA or other ligand that binds IgA. Such antibodies and ligands can be made specific to anti-PAD IgA by, for example, capturing the anti-PAD IgA with PAD and washing away non-anti-PAD IgA prior to adding anti-IgA. Numerous other configurations for isolating such a binding complex in order to achieve specific binding to a target are well known in the art and all of which can be used as an indirect means to make a detection probe specific to a target. Thus, a “detection probe specific for anti-PAD IgA” includes, for example, PAD, a PAD: anti-PAD IgA complex binding agent, an anti-PAD IgA binding agent and an IgA binding agent. The anti-PAD IgA detection probes include binding agents to anti-PAD2 IgA, anti-PAD3 IgA and/or anti-PAD4 IgA.

Accordingly, in one embodiment an exemplary detection probe of the current disclosure which can bind anti-PAD IgA directly is a labeled PAD. A detection probe made specific to anti-PAD IgA by indirect means includes a labeled anti-IgA. These and other exemplary detection probes as well as means for capturing a PAD: anti-PAD IgA complex for specific detection are further described below.

As used herein, the term “reporter tag” refers to a molecule capable of producing a signal indicative of the detection of a biomarker. An exemplary biomarker in the present disclosure includes anti-PAD IgA. Reporter tags can be attached, for example conjugated, to the detection probe through non-covalent or covalent cross-linkage to the detection probe. Non-covalent and covalent immobilization of reporter tags to detection probes can be performed by any means known in the art described in Dennler et al., “Antibody conjugates: from heterogeneous populations to defined reagents,” Antibodies. 4:197-224 (2015). Reporter tags produce various signals, depending on the type of reporter tag. A person skilled in the art appreciates that there are various labels encompassed by reporter tags.

As used herein, the term “label” refers to a molecular entity that emits a signal and can be used as a readout or measurement for detection of an analyte. Various classes of labels exist. Such labels include a fluorophore, an enzyme, a chemiluminscent moiety, a radioactive moiety, an organic dye, a small molecule, a polypeptide or functional fragment thereof. Examples of fluorophores include fluorescent dyes like phycoerytherin (PE), fluorescein isothiocyanate (FITC), tetramethylrhodamine (TRITC), BODIPY and AlexaFluor® dyes. Fluorescent dyes can also include fluorescence resonance energy transfer (FRET)-dyes or time-resolved (TR)-FRET dyes. Fluorophore labels also include fluorescent proteins such as green fluorescent protein (GFP) and cyan fluorescent protein (CFP). Examples of enzyme labels include alkaline phosphatase (AP) or horseradish peroxidase (HRP). When any of the substrates 3,3′5,5′-Tetramethylbenzidine (TMB), 3,3′-Diaminobenzidene (DAB), or 2,2′-azino-bis (3-ethylbenzothiazoline-6-sulphonic acid) (ABTS) are applied to HRP, a colored (chromogenic) or light (chemiluminescent) signal is produced. Radioactive moiety labels include carbon-14 or Tritium. Small molecule labels include biotin, resins such as agarose beads and fluorescently labeled magnetic beads, or nanoparticles such as colloidal gold. Polypeptide or functional fragment labels include Avidin, Streptavidin or NeutrAvidin which have an affinity for biotin. Polypeptide or functional fragment labels also include hemagglutinin (HA), glutathione-S-transferase (GST) or c-myc.

A label of the present disclosure can be conjugated to any of the detection probes identified herein. Conjugation can include non-covalent or covalent cross-linkage as described above. In some configurations, a label conjugated to a detection probe requires an additional substrate or binding agent described above. As an example, an HRP label conjugated to a detection probe requires a substrate, disclosed above, to detect a detection probe. Numerous other configurations for a label are known in the art. The present disclosure includes all label configurations exemplified herein and/or known in the art. In some embodiments, a label configuration can include PE conjugated to a PAD, a PAD: anti-PAD IgA complex binding agent, an anti-PAD IgA or an anti-IgA. In alternative embodiments, a label configuration can include PE conjugated to a specific PAD including, for example, PAD2, PAD3 and/or PAD4. In further embodiments, a label configuration can include a PE conjugated to an anti-PAD IgA including, for example, anti-PAD2 IgA, anti-PAD3 IgA and/or anti-PAD4 IgA.

Methods for detecting, measuring and/or quantifying a signal produced by a label of the present disclosure are well known in the art and include detection of fluorescence, luminescence, chemiluminescence or absorbance, reflectance, transmittance, birefringence or refractive index. Optical methods include imaging methods such as confocal and non-confocal microscopy and non-imaging methods such as microplate readers. In some embodiments, methods of detecting anti-PAD IgA in biological sample can include visualization, quantification or both of a fluorescent, colorimetric or absorbance signal in a biological sample.

In some embodiments of the present disclosure, anti-PAD IgA presence can be detected by immunoassay. Methods and protocols for conducting immunoassays and biophysical protein-interaction assays are well known in the art. See, e.g., Wild D., The Immunoassay Handbook, Elsevier Science, 4^{th} Edition (2013); Fu H., Protein-Protein Interactions, Humana Press, 4^{th} Edition (2004). Exemplary immunoassays include fluorescent immunosorbent assay (FIA), a chemiluminescent immunoassay (CIA), a radioimmunoassay (RIA), multiplex immunoassay, a protein/peptide array immunoassay, a solid phase radioimmunoassay (SPRIA), an indirect immunofluorescence assay (IIF), an enzyme linked immunosorbent assay (ELISA) and a particle based multianalyte test (PMAT), or a Dot Blot assay.

In some embodiments, the ELISA can be a sandwich ELISA. In some embodiments, the sandwich ELISA can include the initial step of immobilizing a purified polypeptide of this disclosure on a solid support as exemplified below. For example, a PAD or antigenic fragment thereof can be immobilized on a wall of a microtiter plate well or of a cuvette. In some embodiments, contacting the sample from the subject with the PAD or antigenic fragment thereof of this disclosure can include exposing the sample to the immobilized PAD or antigenic fragment thereof.

In some embodiments, the ELISA can be a direct ELISA. In some embodiments, the direct ELISA can include the initial step of immobilizing a PAD or antigenic fragment thereof on any of the solid supports disclosed herein. For example, a PAD or antigenic fragment thereof can be immobilized to a wall of a microtiter plate well or of a cuvette. In some embodiments, contacting the sample from the subject with a PAD or antigenic fragment thereof of this disclosure can include exposing the anti-PAD IgA contained in the patient's sample to the immobilized PAD. Any of the immunoassays disclosed herein (see above) and in the art can be used, or modified to be used, in any of the methods disclosed herein.

In some embodiments, anti-PAD IgA can be detected by a particle based multianalyte test. As used herein, the term “particle based multianalyte test (PMAT)” refers to an immunoassay that allows simultaneous measurement of two or more analytes in a single assay. For example, in PMAT, different types of particles are used simultaneously, with each type having immobilized a specific binding partner for a specific molecule species on the surface of its particles. In a solution, the analyte molecules to be detected are bound to their binding partners on the corresponding particle type. The bonds are then detected optically through the addition of a secondary marker that marks all particle-bound analyte molecules of the multiplex assay. A PMAT can be performed using a variety of formats known in the art, such as flow cytometry, a capture sandwich immunoassay, or a competitive immunoassay. For example, using a dual-laser flow-based detection instrument, the binding of analyte fractions, such as autoantibodies, can be detected through the fluorescence of the secondary marker. In some embodiments, the PMAT particle can be a bead. In effecting the PMAT, the presence of one or more autoantibodies specifically associated with an autoimmune disease can be identified, and the patient can be diagnosed with the autoimmune disease that is specifically associated with the autoantibody identified by the PMAT.

In some embodiments, a Dot-Blot or line immunoassay (LIA) can be used to detect anti-PAD IgA in a biological sample. Methods and protocols for dot blot are well known in the art, including estimating polypeptide concentration. See Joint ProteomicS Laboratory (JPSL) of the Ludwig Institute for Cancer Research, Estimating protein concentration by dot blotting of multiple samples, Cold Spring Harbor Protocols, New York (2006).

In some embodiments, the immunoassay can be performed by immobilizing a capture probe to a solid support for a sufficient time to allow binding to occur. A capture probe includes a binding agent that binds to an analyte of interest. With respect to detection of an anti-PAD IgA of this disclosure, a capture probe can be any binding agent that specifically binds to anti-PAD IgA, PAD: anti-PAD IgA complex or anti-PAD IgA. Exemplary capture probes includes, PAD and/or a particular PAD such as PAD2, PAD3 and/or PAD4, as well as antigenic fragments thereof. Other exemplary capture probes include anti-IgA antibodies and functional fragments thereof, anti-IgA binding polypeptides and functional fragments thereof, anti-PAD IgA binding polypeptides, including antibodies, and functional fragments thereof and/or PAD: anti-PAD IgA complex binding polypeptides and functional fragments and binding agents.

Accordingly, in some embodiments, an immunoassay can utilize anti-IgA immobilized to a solid support to capture IgA. In some embodiments, a PAD or antigenic fragment thereof can be immobilized to a solid support to capture anti-PAD IgA. In other embodiments, an anti-PAD IgA binding polypeptide can be immobilized to a solid support to detect IgA. Anti-PAD IgA captured by the above exemplary capture probes can be detected using a detection probe provided in this disclosure.

The immunoassay can further include blocking steps, washing steps and additionally or alternatively, elution steps. Blocking steps can include contacting a solid support of the immunoassay in a blocking buffer for a sufficient time and temperature to allow blocking. Exemplary blocking buffers are identified below as are exemplary solid supports. Washing steps include contacting a solid support of the immunoassay with a washing buffer to remove non-specific binding of polypeptides to the solid support. Exemplary washing buffers are described below. Elution buffers can include any of a variety of elution buffers known in the art or disclosed herein. Elution buffers include, for example, a 0.1 M glycine: HCl solution between pH 2.5 and 3. Polypeptide complexes can be eluted from the solid support of the immunoassay to aid in detection and measurement of, for example, PAD and anti-IgA complexes.

The present disclosure provides a kit which can be used to diagnosis RA, severity of disease and joint erosion. The kit can include a PAD of the present disclosure as exemplified in Tables 1-3 or an antigenic fragment thereof. In some embodiments, a PAD or antigenic fragment thereof can include any mammalian PAD as provided herein. Exemplary PADS include, for example, PAD2, PAD3 and PAD4.

The kit can include any of the detection probes provided herein as well as others well known in the art. For example, a detection probe can include an antibody or a ligand. A detection probe can be immobilized on a solid support. It should be noted that the term “immobilized” is used interchangeably with “attached” and both terms are intended to include covalent and non-covalent attachment, unless indicated otherwise, either explicitly or by context. In some embodiments, a PAD or antigenic fragment thereof is immobilized to a solid support. h

As provided herein and exemplified with respect to the methods of this disclosure, a kit of this disclosure can include a reporter tag. Reporter tags function to produce a signal for detection of a biomarker. Reporter tags can be attached, for example, to any of the detection probes used herein through non-covalent or covalent cross-linkage. As exemplified with respect to the methods of this disclosure, a kit can include any of the labels described or exemplified herein. For example, a label of the kit can include a fluorophore, an enzyme, a chemiluminescent moiety, a radioactive moiety, an organic dye, a small molecule, a polypeptide or functional fragment thereof. In some embodiments, a label of the kit includes PE. In some embodiments, a label of the kit includes FITC. In some embodiments, a label of the present disclosure is conjugated to a detection probe of the disclosure as exemplified above.

A kit can include any solid support provided herein or identified in the art. As used herein, the terms “solid support,” “solid surface” and other grammatical equivalents refer to any material that is appropriate for or can be modified to be appropriate for the attachment of PAD or an antigenic fragment thereof of this disclosure. Possible materials include, without limitation, glass and modified or functionalized glass, plastics (including acrylics, polystyrene, methylstyrene, polyurethanes, Teflon™, etc.), paramagnetic materials, thoria sol, carbon graphite, titanium oxide, latex or cross-linked dextrans such as Sepharose, cellulose polysaccharides, nylon or nitrocellulose, ceramics, resins, silica or silica-based materials including silicon and modified silicon, carbon metals, inorganic glasses, optical fiber bundles, and a variety of other polymers. In some embodiments, the solid supports can be located in microtiter well plates (e.g., a 96-well, 384-well or 1536-well plate). In some embodiments, the solid supports can be located within a flow cell or flow cell apparatus (e.g., a flow cell on a Biacore™M chip or a protein chip).

In some embodiments, the solid support can be a bead, microsphere, particle, membrane, chip, slide, well, and test tube. Beads include microspheres or particles. By “microspheres” or “particles” or grammatical equivalents herein is meant small, discrete, non-planar particles in the micrometer or nanometer dimensions. In some embodiments the bead can be spherical, in other embodiments the bead is irregular. Alternatively or additionally, the beads can be porous. The bead sizes range from nanometers to millimeters with beads from about 0.2 to about 200 microns being preferred in some embodiments. In other embodiments, bead size can range from about 0.5 to about 5 microns. In some embodiments, beads smaller than 0.2 microns and larger than 200 microns can be used. In some embodiments, the solid support can include an array of wells or depressions in a surface. This can be fabricated as is known in the art using a variety of techniques, including, photolithography, stamping techniques, molding techniques and microetching techniques. As will be appreciated by those skilled in the art, the technique used will depend on the composition and shape of the array substrate.

In some embodiments, the solid support can include a patterned surface suitable for immobilization of purified proteins in an ordered pattern (e.g., a protein chip). A “patterned surface” refers to an arrangement of different regions in or on an exposed layer of a solid support. For example, one or more of the regions can be features where one or more purified proteins are present. The features can be separated by interstitial regions where purified proteins are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. Exemplary patterned surfaces that can be used in the methods and compositions set forth herein are described in U.S. Pat. App. Publ. No. 2008/0280785 A1, U.S. Pat. App. Publ. No. 2004/0253640 A1, U.S. Pat. App. Publ. No. 2003/0153013 A1 and International Publication No. WO 2009/039170 A2.

In some embodiments, a solid support can have attached to its surface a PAD or an antigenic fragment thereof or anti-IgA. Any PAD exemplified by, for example, Tables 1-3, including antigenic fragments thereof can be attached to a solid support. In some embodiments, any PAD or antigenic fragment thereof of the present disclosure can be immobilized to a solid support via a linker molecule. In some embodiments, all that is required is that molecules, such as any PAD or antigenic fragment thereof of the present disclosure, remain immobilized or attached to the support under the conditions in which it is intended to use the support, for example, in applications requiring antibody binding or detection.

A kit can include a positive control. In some embodiments, a positive control can be a sample containing a detectable amount of anti-PAD IgA or levels above the threshold. In some embodiments, a positive control can be obtained from a diseased subject who has levels of anti-PAD IgA above threshold. Additionally or alternatively, a positive control can contain anti-PAD IgA synthesized in vitro using any of the methods described herein. In other embodiments, the kit can include a negative control. A negative control can be a sample containing no detectable amount of anti-PAD IgA or levels below the threshold. In some embodiments, a negative control can be obtained from a healthy control individual or can be synthesized in vitro. For example, a negative control can include water or buffer.

The kit or the disclosure can further include one or more ancillary reagents. As used herein, “ancillary reagents” refer to a substance, mixture, material or component that is useful to carry out an intended purpose of the kit. Ancillary reagents can include a reagent, including a conjugation reagent, a buffer, standard, positive control, label, instructions and the like.

In some embodiments, a reagent of the kit of the present disclosure can include any conjugation reagent known in the art, including covalent and non-covalent conjugation reagents. Covalent conjugation reagents can include any chemical or biological reagent that can be used to covalently immobilize a polypeptide of this disclosure on a surface. Covalent conjugation reagents can include a carboxyl-to-amine reactive group such as carbodiimides such as EDC or DCC, an amine reactive group such as N-hydroxysuccinimide (NHS) ester or imidoesters, a sulfhydryl-reactive crosslinker such as maleimides, haloacetyls, or pyridyl disulfides, a carbonyl-reactive crosslinker groups such as, hydrazides or alkoxyamines, a photoreactive crosslinker such as aryl azides or dizirines, or a chemoselective ligation group such as a Staudinger reaction pair. Non-covalent immobilization reagents can include any chemical or biological reagent that can be used to immobilize a polypeptide of this disclosure non-covalently on a surface, such as affinity tags such as biotin or capture reagents such as streptavidin or anti-tag antibodies, such as anti-His6 or anti-Myc antibodies.

The kits of this disclosure can include combinations of conjugation reagents. Such combinations include, e.g., EDC and NHS, which can be used, e.g., to immobilize a protein of this disclosure on a surface, such as a carboxylated dextrane matrix (e.g., on a BIAcore™ CM5 chip or a dextrane-based bead). Combinations of conjugation reagents can be stored as premixed reagent combinations or with one or more conjugation reagents of the combination being stored separately from other conjugation reagents.

In other embodiments, a reagent of the kit can include a reagent such as a coating buffer. A coating buffer can include sodium carbonate-sodium hydroxide or phosphate. In some embodiments, the coating buffer can be 0.1M NaHCO₃ (e.g., about pH 9.6).

In some embodiments, a reagent of a kit can include a washing buffer. A washing buffer can include tris (hydroxymethyl) aminomethane (Tris)-based buffers like Tris-buffered saline (TBS) or phosphate buffers like phosphate-buffered saline (PBS). Washing buffers can be composed of detergents, such as ionic or non-ionic detergents. In some embodiments, the washing buffer can be a PBS buffer at about pH 7.4 including Tween®20 at about 0.05%. In other embodiments, the washing buffer can be the BIO-FLASH™ Special Wash Solution (Inova Diagnostics, Inc., San Diego, CA).

In some embodiments, a reagent of the kit can include a dilution buffer. Any dilution buffer known in the art can be included in the kit of the present disclosure. Typical dilution buffers include a carrier protein such as bovine serum albumin (BSA) and a detergent such as Tween®20. In some embodiments, the dilution buffer can be PBS at about pH 7.4 including BSA at about 1% BSA and Tween®20 at about 0.05%.

In some embodiments, a reagent can include a detection or assay buffer. Any detection or assay buffer known in the art can be included in the kit of the present disclosure. The detection or assay buffer can be a colorimetric detection or assay buffer, a fluorescent detection or assay buffer or a chemiluminescent detection or assay buffer. Colorimetric detection or assay buffers include PNPP (p-nitrophenyl phosphate), ABTS (2,2′-azino-bis (3-ethylbenzothiazoline-6-sulphonic acid)) or OPD (o-phenylenediamine). Fluorescent detection or assay buffers include QuantaBlu™ or QuantaRed™ (Thermo Scientific, Waltham, MA). Chemiluminescent detection or assay buffers can include luminol or luciferin. Detection or assay buffers can also include a trigger such as H₂O₂ and a tracer such as isoluminol-conjugate. In some embodiments, the detection reagent can include one or more BIO-FLASH™ Trigger solutions (Inova Diagnostics, Inc., San Diego, CA). In some embodiments, a reagent of the kit of the present disclosure can include solutions useful for calibration or testing.

In some embodiments, a reagent of the kit can include a stop solution. Any stop solution known in the art can be included in a kit of this disclosure. The stop solutions of this disclosure terminate or delay the further development of the detection reagent and corresponding assay signals. Stop solutions can include, e.g., low-pH buffers (e.g., glycine-buffer, pH 2.0), chaotrophic agents (e.g., guanidinium chloride, sodium-dodecylsulfate (SDS)) or reducing agents (e.g., dithiothreitol, β-mecaptoethanol), or the like.

In some embodiments, a reagent of the kit of this disclosure can include cleaning reagents. Cleaning reagents can include any cleaning reagent known in the art. In some embodiments, the cleaning reagents can be the cleaning reagents recommended by the manufacturers of the automated assay systems. In some embodiments, the cleaning reagents can include the BIO-FLASH™ System Rinse or the BIO-FLASH™ System Cleaning solutions (Inova Diagnostics, Inc., San Diego, CA).

A detection probe of the kit can include any of the detection probes described above. In brief, a detection probe of the kit can include antibodies and ligands. Thus, a “detection probe specific for anti-PAD IgA” includes, for example, PAD, a PAD: anti-PAD IgA complex binding agent, an anti-PAD IgA binding agent and an IgA binding agent. The anti-PAD IgA detection probes include binding agents to anti-PAD2 IgA, anti-PAD3 IgA and/or anti-PAD4 IgA.

A detection probe of the kit can be conjugated to any of the labels previously disclosed herein. For example, a detection probe can be conjugated to a fluorophore, an enzyme, a chemiluminscent moiety, a radioactive moiety, an organic dye, a small molecule, a polypeptide or functional fragment thereof. Examples of fluorophores include fluorescent dyes like phycoerytherin (PE), fluorescein isothiocyanate (FITC), tetramethylrhodamine (TRITC), BODIPY and AlexaFluor® dyes. Fluorescent dyes can also include fluorescence resonance energy transfer (FRET)-dyes or time-resolved (TR)-FRET dyes. Fluorophore labels also include fluorescent proteins such as green fluorescent protein (GFP) and cyan fluorescent protein (CFP). Examples of enzyme labels include alkaline phosphatase (AP) or horseradish peroxidase (HRP). When any of the substrates 3,3′5,5′-Tetramethylbenzidine (TMB), 3,3′-Diaminobenzidene (DAB), or 2,2′-azino-bis (3-ethylbenzothiazoline-6-sulphonic acid) (ABTS) are applied to HRP, a colored (chromogenic) or light (chemiluminescent) signal is produced. Radioactive moiety labels include carbon-14 or Tritium. Small molecule labels include biotin, resins such as agarose beads and fluorescently labeled magnetic beads, or nanoparticles such as colloidal gold. Polypeptide or functional fragment labels include Avidin, Streptavidin or NeutrAvidin which have an affinity for biotin. Polypeptide or functional fragment labels also include hemagglutinin (HA), glutathione-S-transferase (GST) or c-myc.

In some embodiments, the kit provided in this disclosure can include a component suitable for collecting a biological sample. A component can include collection tubes, columns, syringes, needles and the like. In some embodiments, the kit can include instructions for using the components of the kit. Instructions can be in any form, inside or outside of the kit. The instructions provide details regarding protocol and analytical techniques.

In some embodiments, a kit of the disclosure can include an instrument to an automated assay system. Automated assay systems can include systems by any manufacturer. In some embodiments, the automated assay systems can include, e.g., the BIO-FLASH™, the BEST 2000™, the DS2™, the ELx50 WASHER, the ELx800 WASHER, the ELx800 READER, and the Autoblot S20™ (Inova Diagnostics, Inc., San Diego, CA). In other embodiments, an instrument of the kit can be a detection instrument. A detection instrument can include any detection instrument in the art. Detection instruments are capable of detecting or measuring a label of the reporter tags of the present disclosure. Thus, detection instruments are capable of detecting or measuring fluorescence, luminescence, chemiluminescence or absorbance, reflectance, transmittance, birefringence or refractive index. In some embodiments, detection instruments can include confocal and non-confocal microscopy, a microplate reader, a flow cytometer and the like.

Components of a kit of the disclosure can be in varying physical states. For example, some or all of the components can be lyophilized or in aqueous solution or frozen. Such components include a PAD, a detection probe, and ancillary reagents. Ancillary reagents include immobilization buffer, incubation buffer, washing buffer, dilution buffer, detection or assay buffer and blocking buffer. A person skilled in the art recognizes that there are various types of incubation, washing, detection and blocking buffers.

A kit of this disclosure can be tailored to specific assay technologies. In some embodiments, a kit can be tailored to assay technologies exemplified herein. For example, in some embodiments, the kits can be a FIA kit, a CIA kit, a RIA kit, a multiplex immunoassay kit, a protein/peptide array immunoassay kit, a SPRIA kit, an IIF kit, an ELISA, a PMAT kit, or a Dot Blot kit. In some embodiments, the ELSA kits can include a washing buffer, a sample diluents, a secondary antibody-enzyme conjugate, a detection reagent and a stop solution. In some embodiments, the Dot Blot kits can include a washing buffer, a sample diluents, a secondary antibody-enzyme conjugate, a detection reagent, and a stop solution. In some embodiments, the CIA kit can include a washing buffer, a sample diluent, a tracer (e.g., isoluminol-conjugate) and a trigger (e.g., H₂O₂). In some embodiments, the multiplex kit can include a washing buffer, a sample diluent and a secondary antibody-enzyme conjugate. In some embodiments, the kits can be tailored to the Luminex platform and include, as an example, xMAP® beads.

A kit can be used to diagnose RA, severity of disease or joint erosion by providing a means for detecting anti-IgA bound to PAD or an antigenic fragment thereof. A kit can detect anti-IgA by any of the methods disclosed herein (see above). Complexes of anti-PAD IgA and a PAD, or antigenic fragment thereof, can have a stoichiometry of one to one or more than one to one anti-PAD IgA. In some embodiments, the complexes can have one anti-PAD IgA antibody per PAD or antigenic fragment thereof. In some embodiments, the complexes can have two anti-PAD IgA per PAD or antigenic fragment thereof. In some embodiments, the complexes can have more than two anti-PAD IgA per PAD or antigenic fragment thereof. Methods for measuring binding stoichiometries of two antigens are well known in the art and include, e.g., isothermal titration calorimetry (ITC) and ultracentrifugation.

In some embodiments, the complexes of anti-PAD IgA and PAD, or antigenic fragment thereof, can be a plurality of complexes with identical stoichiometry. For example, all complexes in the plurality of complexes have one anti-PAD IgA per purified PAD or antigenic fragment thereof. In some embodiments, the complexes of anti-PAD IgA and PAD or antigenic fragment thereof, can be a plurality of complexes with different stoichiometries. For example, some complexes in the plurality of complexes can have one anti-PAD IgA per PAD or antigenic fragment thereof and some other complexes in the plurality of complexes can have more than one anti-PAD IgA per PAD or antigenic fragment thereof.

In some embodiments, a PAD or antigenic fragment thereof can be bound by anti-PAD IgA with higher affinity. In some embodiments, anti-PAD IgA binding sites can be bound by anti-PAD IgA with more than 2-fold, more than 3-fold, more than 4-fold, more than 5-fold, more than 8-fold, more than 10-fold, more than 15-fold, more than 20-fold, more than 25-fold, more than 50-fold, more than 100-fold, more than 300-fold, more than 1,000-fold, more than 3,000-fold, more than 10,000-fold, more than 30,000-fold, or more than 100,000-fold greater binding affinity. Greater binding affinities are evidenced by lower dissociation constants (K_{D}s) for anti-PAD IgA-PAD complex or by higher association constants (K_{A}s) for the respective anti-PAD IgA and PAD. In some embodiments, the dissociation constants for (K_{D}s) for the anti-PAD IgA-PAD complexes can be less than 1 mM, less than 300 nM, less than 100 nM, less than 30 nM, less than 10 nM, less than 3 nM, less than 1 nM, less than 300 pM, less than 100 pM, less than 30 pM, less than 10 pM, less than 3 pM, or less than 1 pM. Methods for measuring binding affinities of antibodies to antigens are well known in the art and include ELISA, isothermal titration calorimetry (ITC) and surface plasmon resonance (SPR).

### EXAMPLE I

### Detection of Anti-PAD4, Anti-PAD2 IgA and Joint Erosion in Rheumatoid Arthritis Patients

This example illustrates the use of anti-PAD4 IgA and anti-PAD2 IgA as biomarkers for the detection of joint erosion in rheumatoid arthritis (RA).

Anti-PAD4 IgA and anti-PAD2 IgA were measured using a particle-based multianalyte test (PMAT, Inova Diagnostics, San Diego, US). For this test, human recombinant full-length PAD4 polypeptide (Cayman Chemical, Ann Arbor, MI; cat no. 10500) and human recombinant full-length PAD2 polypeptide (John Hopkins University, Baltimore, MD) were coupled to paramagnetic beads with unique signatures. The coupling procedure includes bead activation, antigen coupling and bead blocking.

Bead activation was performed by incubating the beads for 30 min at room temperature with an activation buffer. Once the beads were activated, they were incubated with the antigen for 1 hour at room temperature in coupling buffer at a concentration of 22.2 μg of antigen/million of beads for PAD4 and 10 μg of antigen/million for PAD2. Finally, the beads were blocked for 1 hour at room temperature with PBS-TBN buffer. Once the beads were coupled, they were resuspended in a PBS-based assay resuspension buffer at a concentration of 1500 beads/test.

Measurement of anti-PAD4 IgA and anti-PAD2 IgA was performed as follows. First, sera from 41 RA patients with known erosion status were diluted 1:7 in Hemosil Rinse Solution (Inova Diagnostics, San Diego, CA). Next, PAD4 and PAD2 coupled beads were incubated for 9.5 min at 37° C. with patients' serum and assay buffer. After three washes with Hemosil Rinse Solution, the beads were then incubated for 9.5 min at 37° C. with a phycoerythrin (PE)-labeled anti-human IgA detector (Inova Diagnostics, San Diego, CA) at a concentration of 5μg/mL in QUANTA Flash Diluent (Inova Diagnostics, San Diego, CA). After incubation, beads were washed again in Hemosil Rinse Solution and the particles were analyzed through digital imaging technology. Finally, the Median Fluorescence Intensity (MFI) was calculated on the particles.

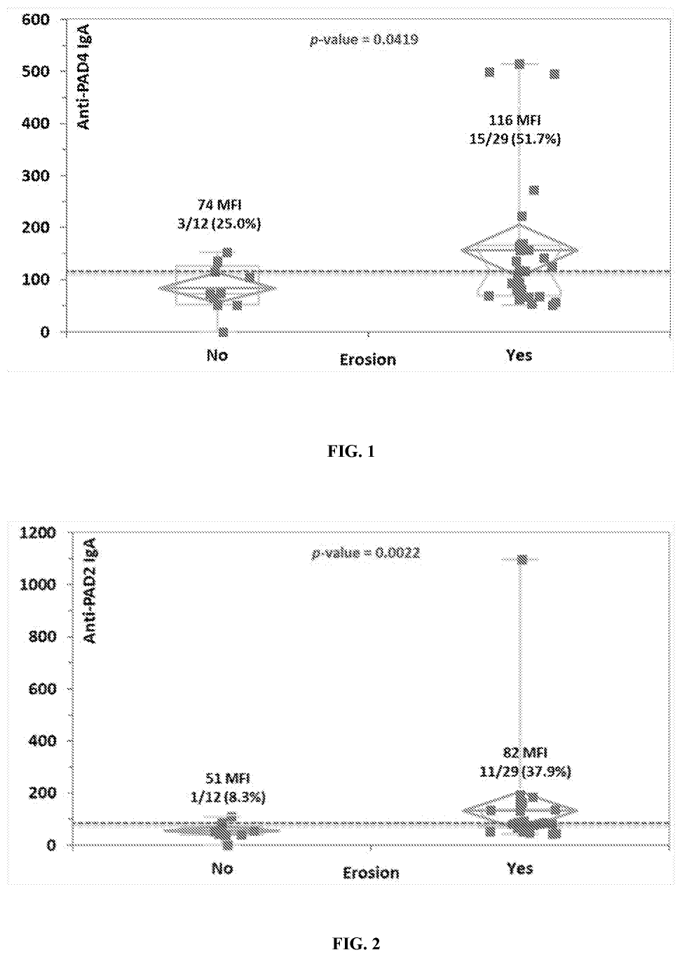

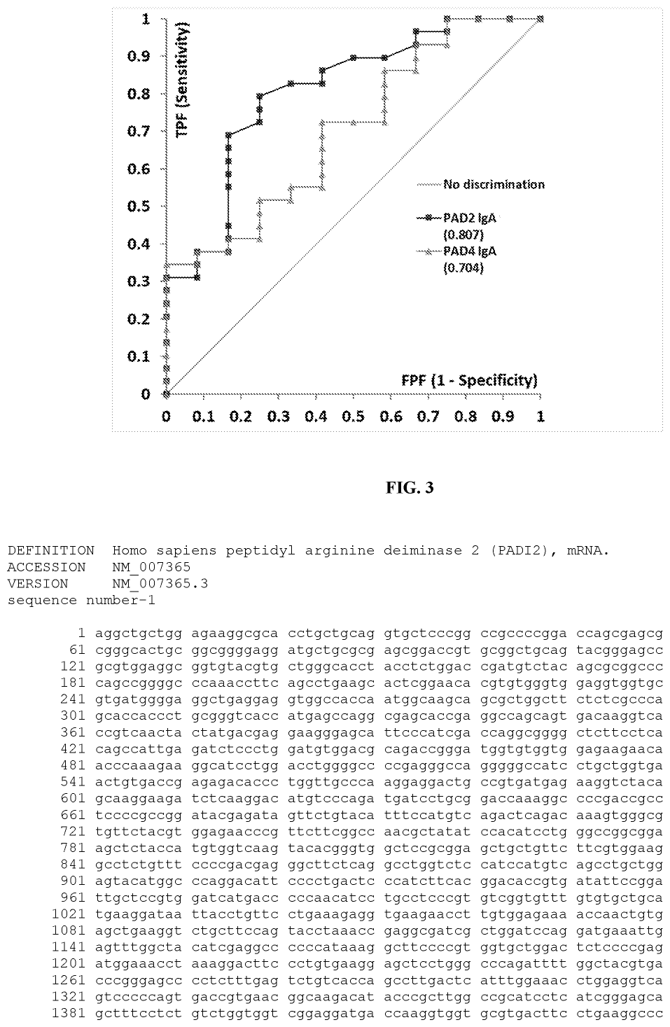

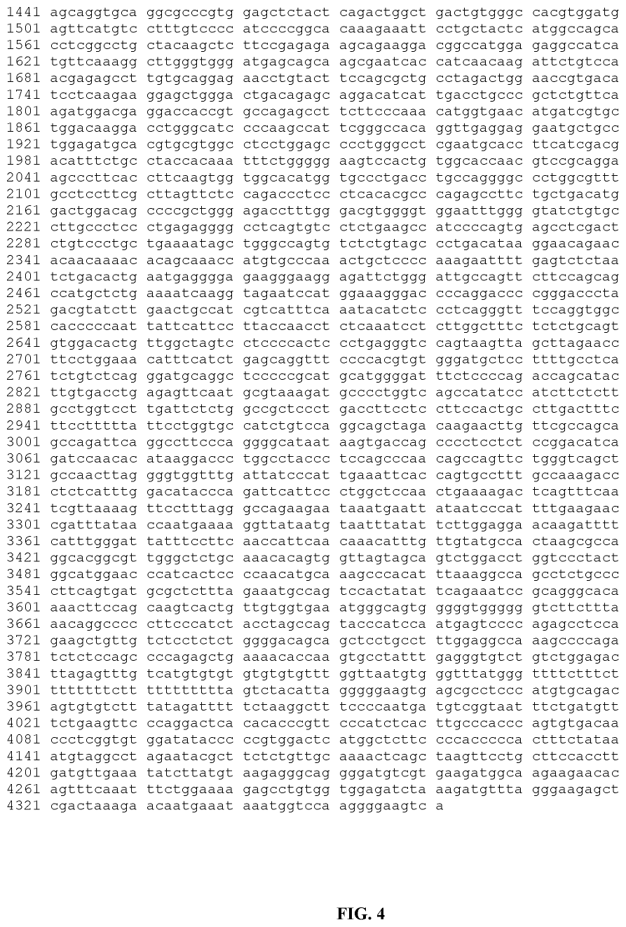

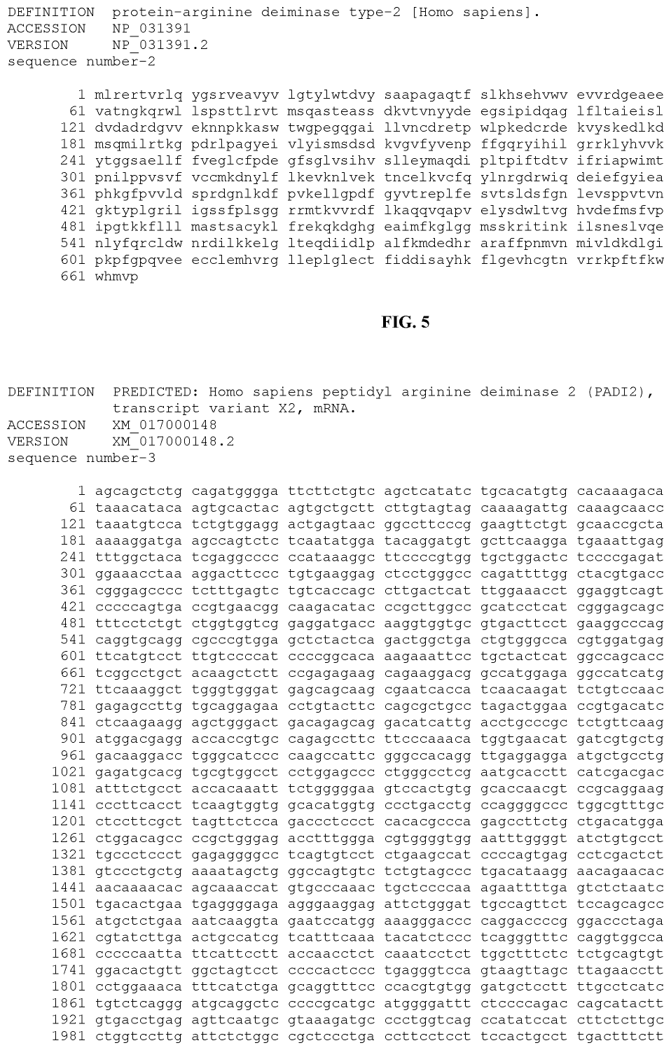

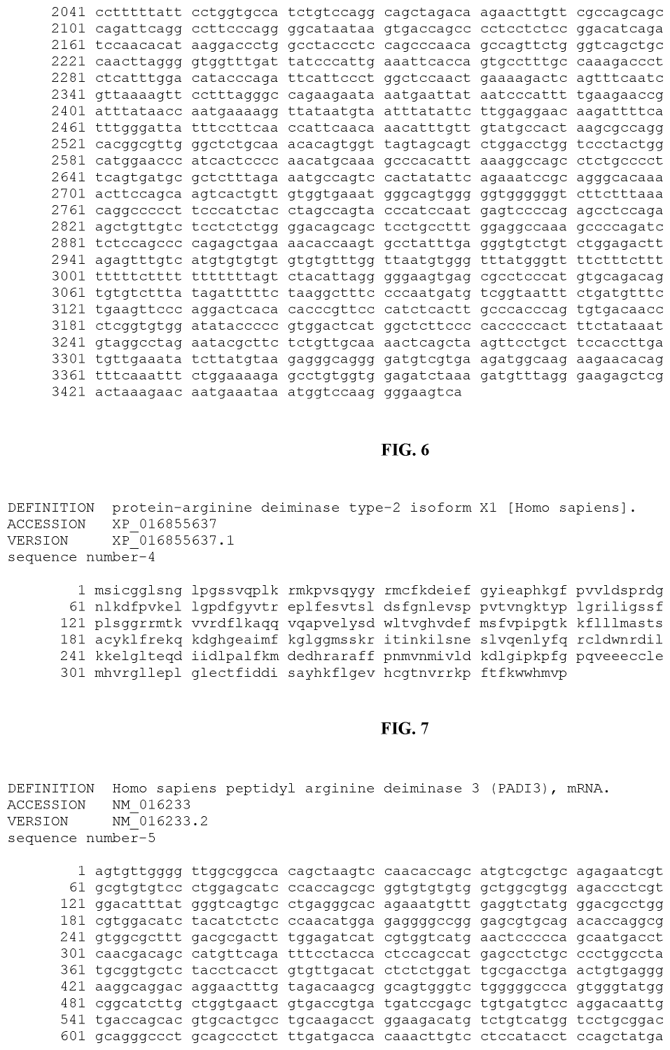

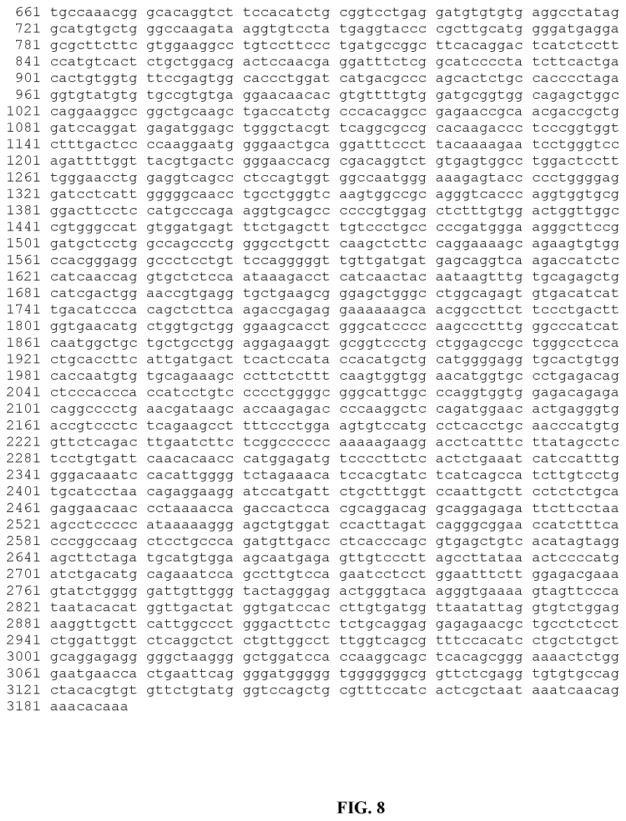

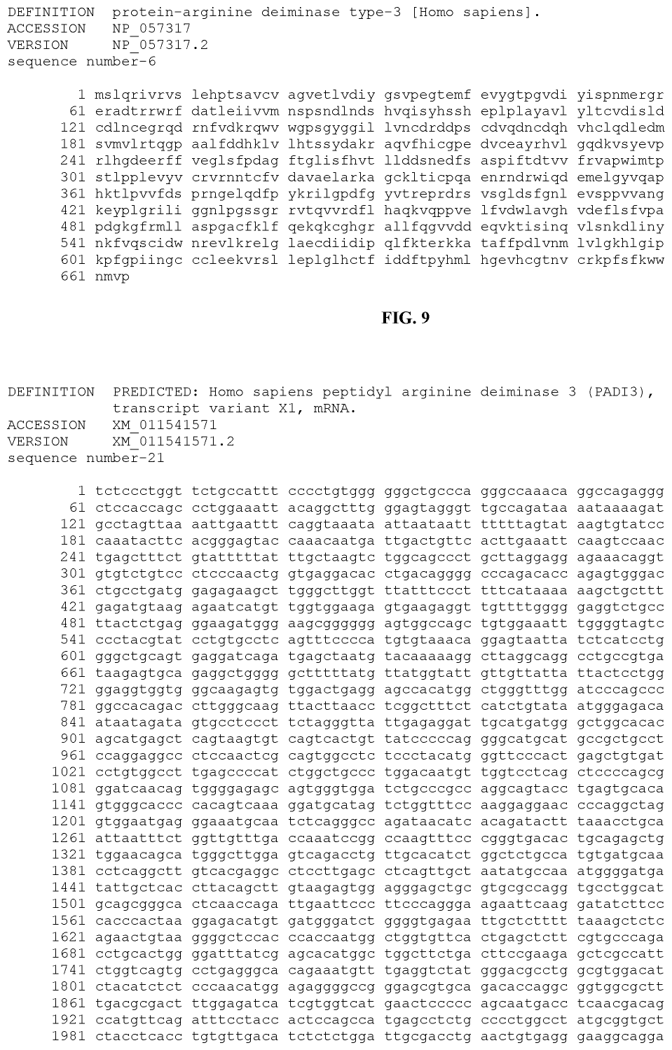

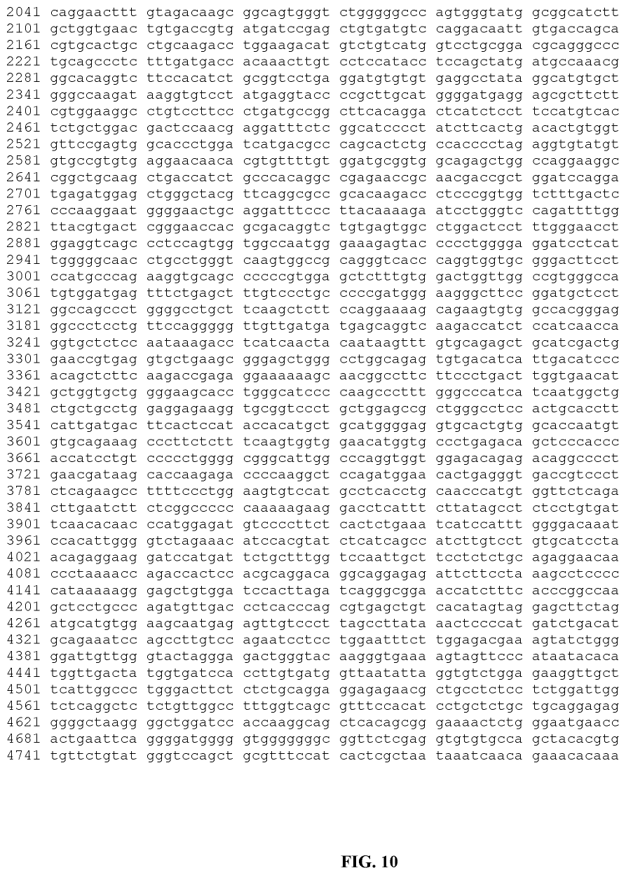

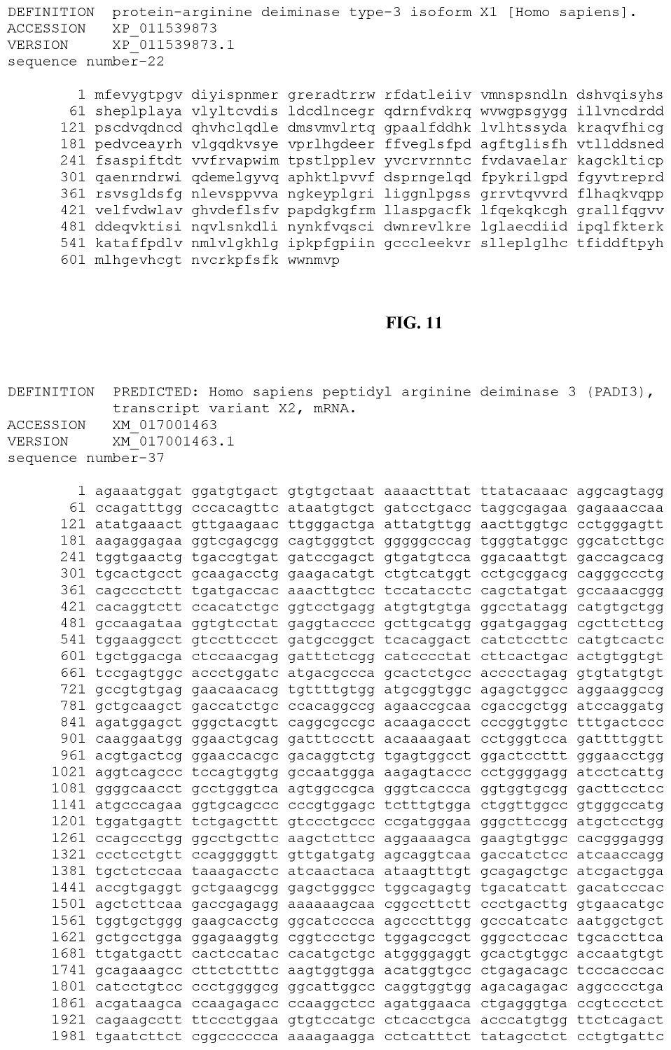

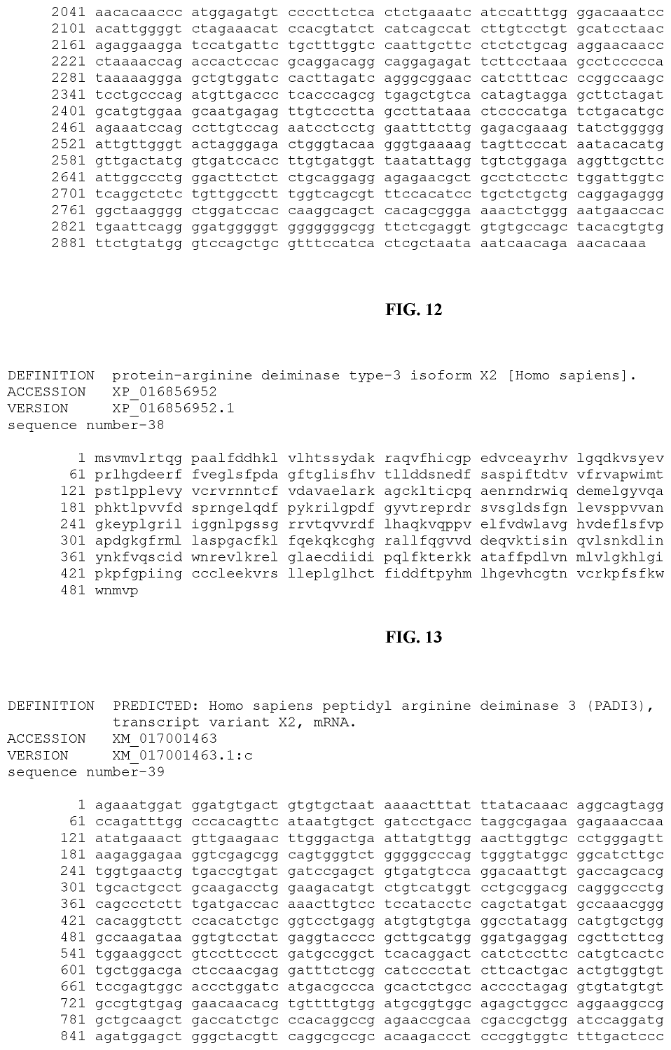

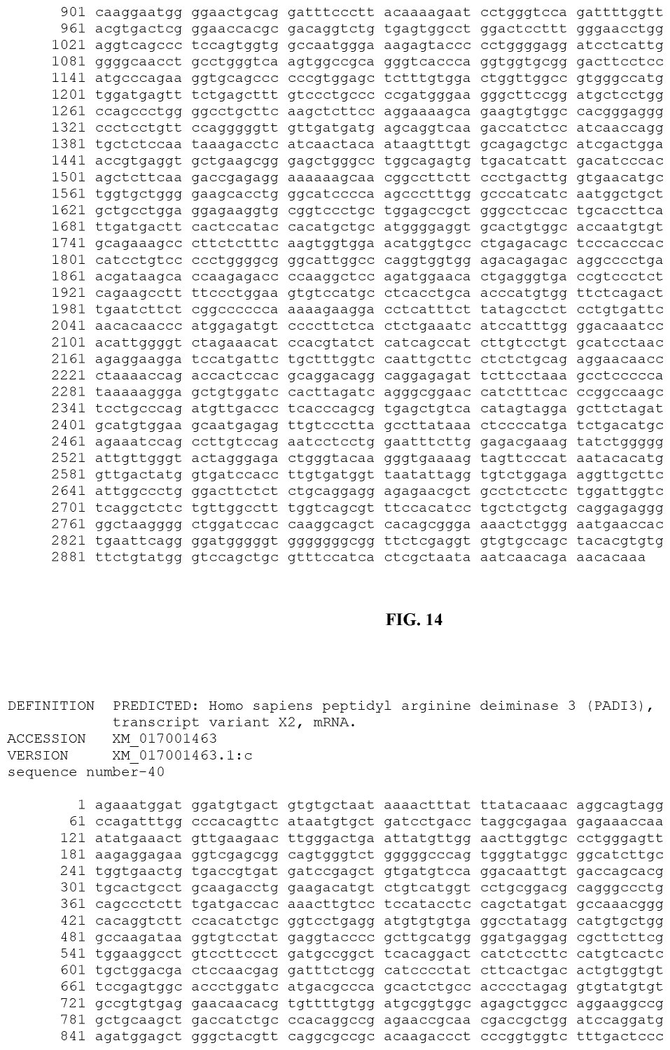

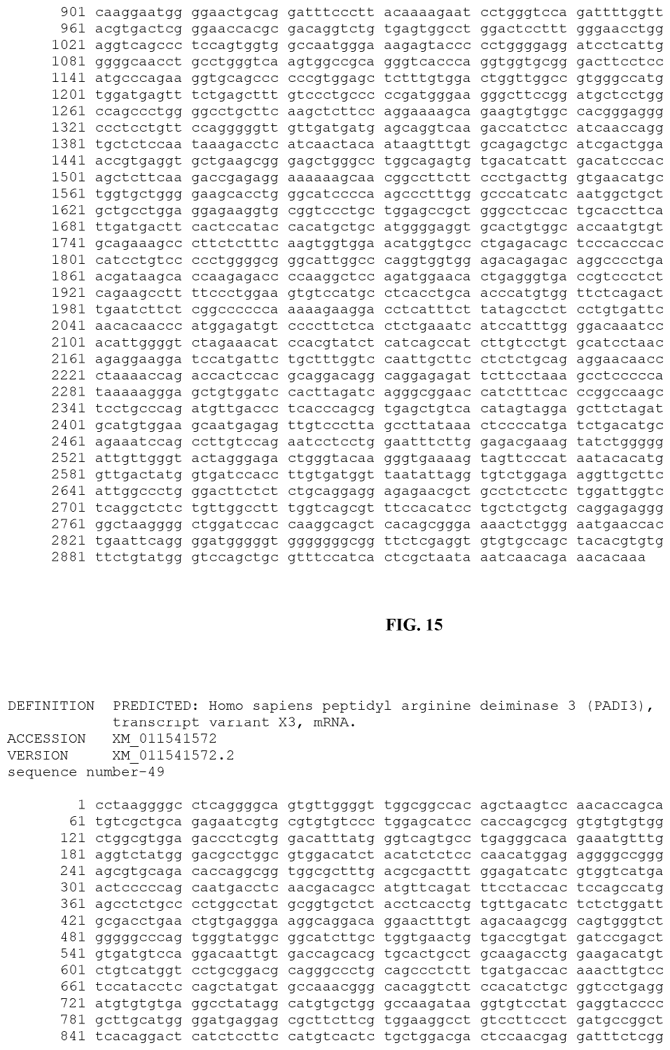

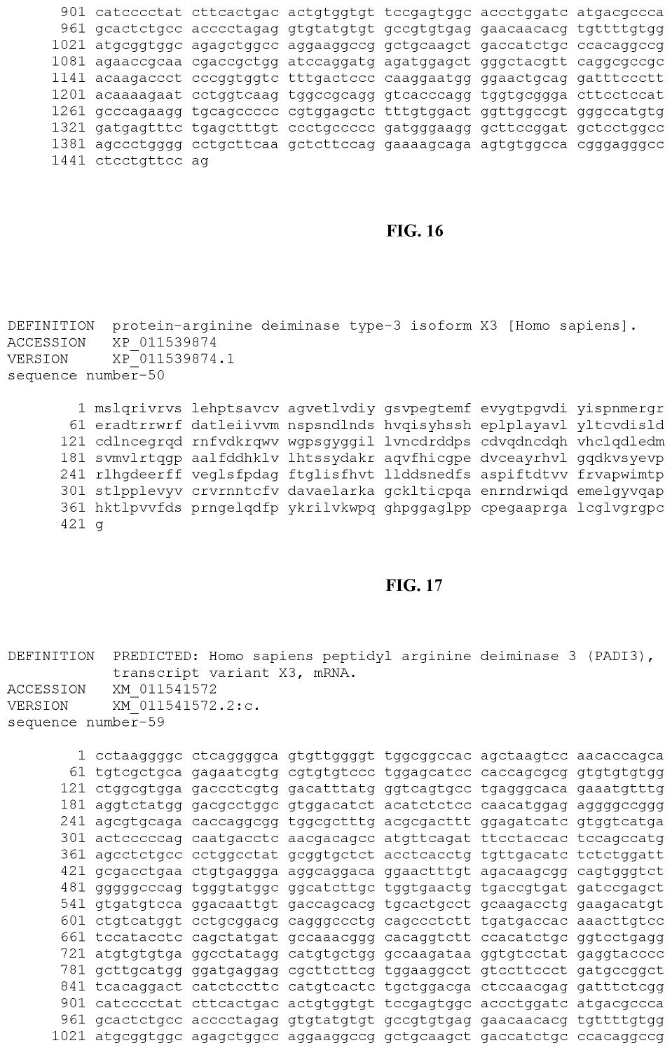

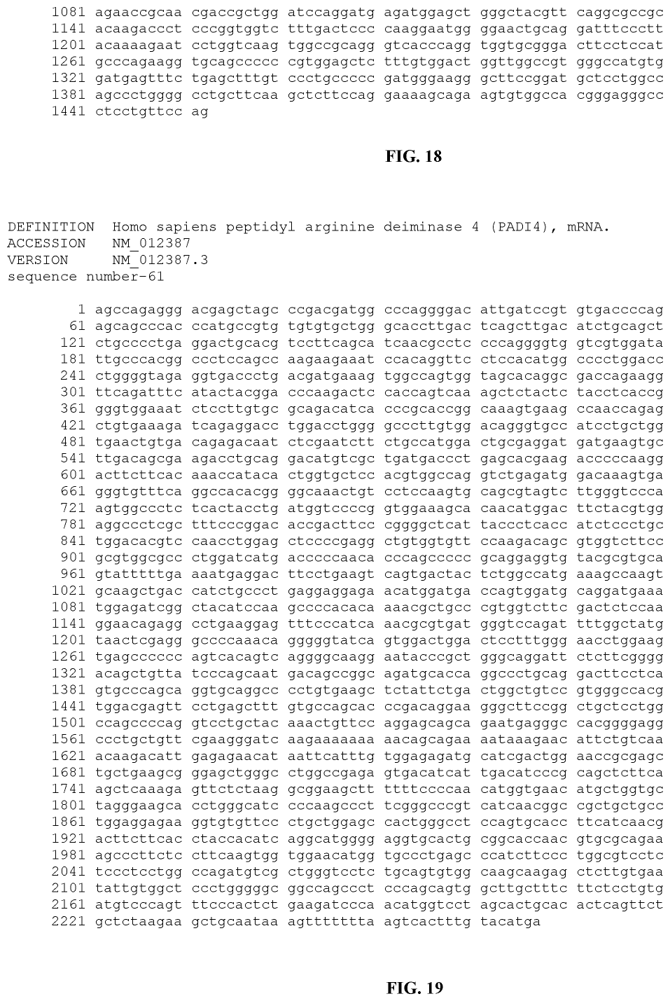

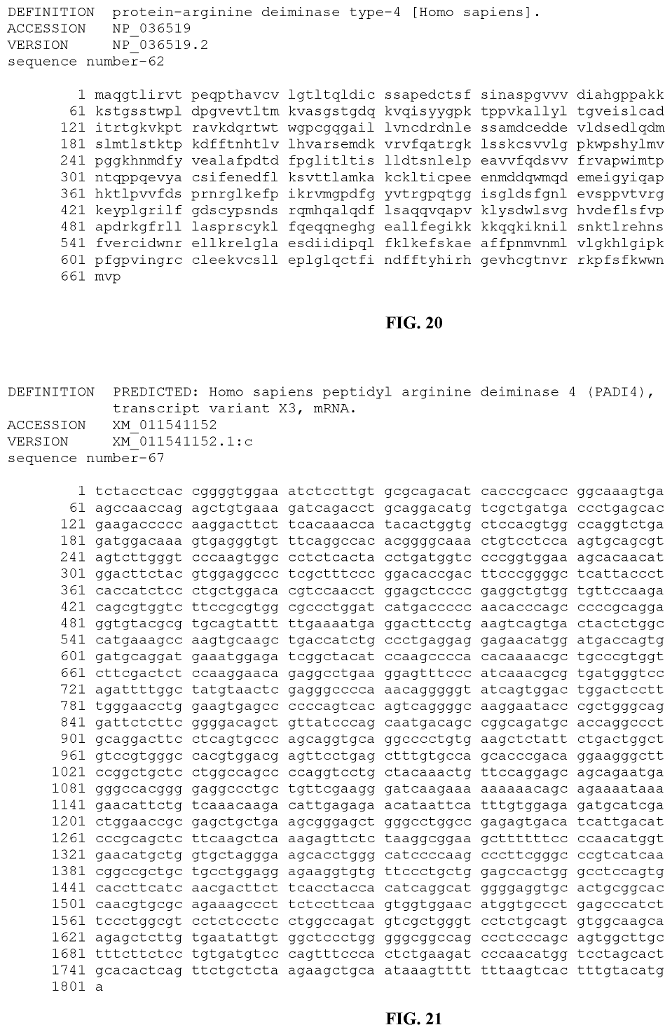

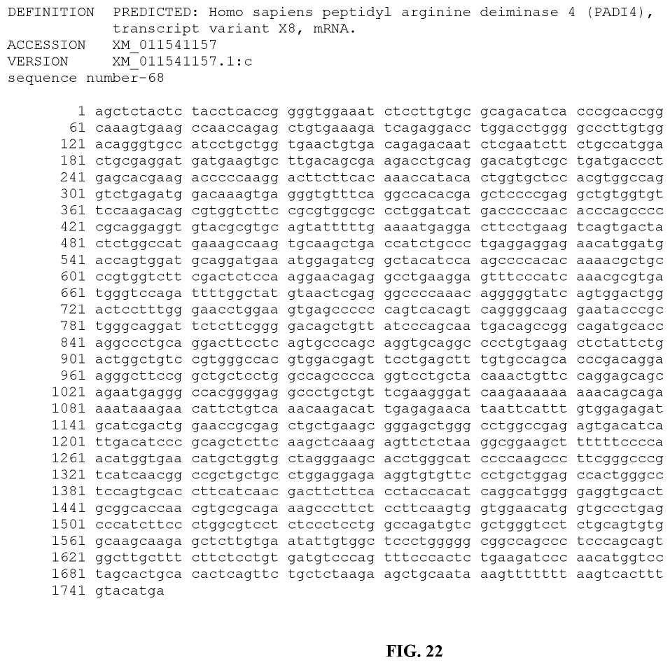

Anti-PAD4 IgA and anti-PAD2 IgA were significantly higher in RA patients with erosions compared to individuals without erosions (p=0.0022 and p=0.0419, respectively). See, FIG. 1 and FIG. 2. Discrimination between the populations of subjects with and without erosive disease reported an Area Under the Curve (AUC) of 0.704 (95% CI 0.529-0.879) for anti-PAD4 IgA. See FIG. 1. With an assay preliminary cut-off of 88 MFI determined by Receiver Operating Characteristic (ROC) curve analysis, anti-PAD2 IgA positive patients were 6.7 (95% CI 0.9-45.6) times more likely to have erosive disease. With an assay preliminary cut-off of 116 MFI, anti-PAD4 IgA positive patients were 3.2 (95% CI 0.8-13.4) times more likely to have erosive disease.

In conclusion, this example demonstrates that anti-PAD4 IgA and anti-PAD2 IgA are indicative of erosive disease in RA. These data further demonstrate that anti-PAD4 IgA and anti-PAD2 IgA represent useful biomarkers for patient stratification.

### EXAMPLE II

### Detection of Anti-PAD4 IgA, IgG and IgM in Rheumatoid Arthritis Patients

This example illustrates the use of anti-PAD4 IgA, IgG and IgM as biomarkers for the detection of joint erosion in rheumatoid arthritis (RA).

Bead activation was performed by incubating the beads for 30 min at room temperature with an activation buffer. Once the beads were activated, they were incubated with the antigen for 1 hour at room temperature in coupling buffer at a concentration of 22.2 μg of antigen/million of beads for PAD4 and 10 μg of antigen/million for PAD2. Finally, the beads were blocked for 1 hour at room temperature with a PBS-TBN buffer. Once the beads were coupled, they were resuspended in a PBS-based assay resuspension buffer at a concentration of 1500 beads/test.

Measurement of anti-PAD4 IgA, IgG and IgM was performed as follows. First, sera from 62 RA patients with known erosion status were diluted 1:7 in Hemosil Rinse Solution (Inova Diagnostics, San Diego, CA). Next, PAD4 coupled beads were incubated for 9.5 min at 37° C. with patients' serum and assay buffer. After three washes with Hemosil Rinse Solution, the beads were then incubated for 9.5 min at 37° C. with a PE-labeled anti-human IgA, IgG or IgM detector (Inova Diagnostics, San Diego, CA) at the concentrations of 5, 1 and 5μg/mL, respectively, and diluted in QUANTA Flash Diluent (Inova Diagnostics, San Diego, CA). After incubation, beads were washed again in Hemosil Rinse Solution and the particles were analyzed through digital imaging technology. Finally, the Median Fluorescence Intensity (MFI) was calculated on the particles.

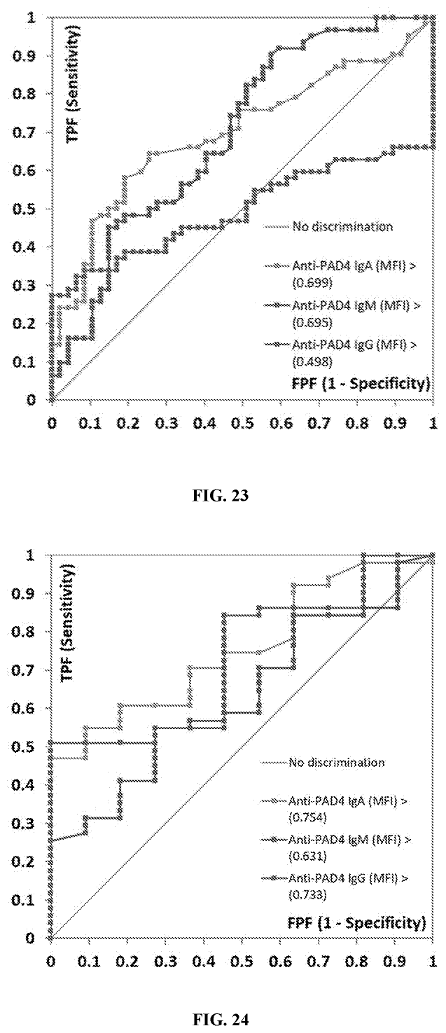

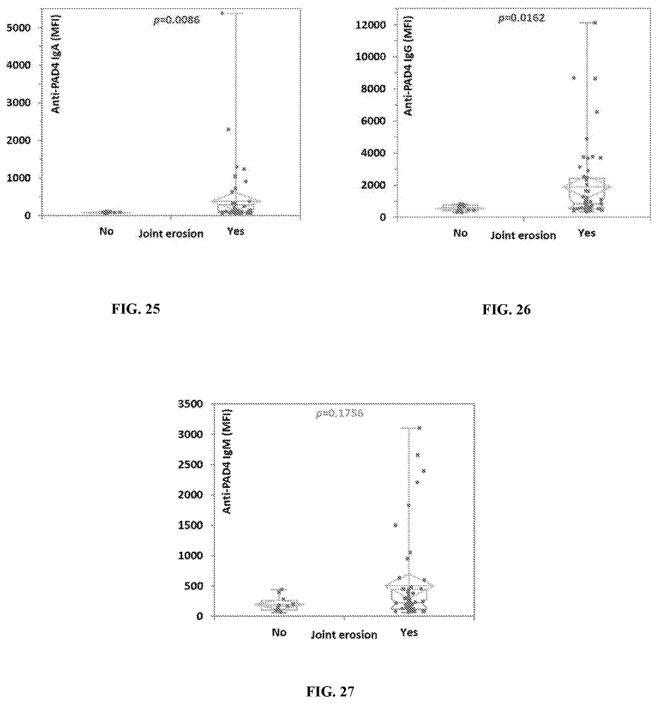

Anti-PAD4 IgA and IgM, but not IgG, were significantly higher in RA patients with erosions compared to individuals without erosions (p=0.0004, p=0.0005 and p=0.9707, respectively). ROC analysis showed higher AUC values for the discrimination between RA and controls for anti-PAD4 IgA and IgM [0.70 (95% CI 0.60-0.80) and 0.70 (95% CI 0.59-0.80), respectively] than for anti-PAD4 IgG [0.50 (95% CI 0.39-0.61)]. At the relevant diagnostic area (>90% specificity), IgG outperformed the other two markers. See, FIG. 23. Discrimination for erosive disease was observed with anti-PAD4 IgA, followed by IgG and IgM. See, FIG. 24. Spearman correlation analysis showed moderate significant association between IgA and IgG (Spearman's rs-0.45, p<0.0001) and between IgA and IgM (Spearman's rs-0.45, p<0.0001), and a weak correlation between IgG and IgM (Spearman's rs-0.27, p-0.0053). When subjects with RA were stratified by presence or absence of erosive disease, higher titers of the three isotypes were observed in patients with erosive disease compared to individuals without erosions. However, this association was only significant for anti-PAD4 IgA and IgG (p=0.0086 and p=0.0162) (See, FIG. 25 and FIG. 26) but not anti-PAD4 IgM (p=0.1756) (See, FIG. 27).

In conclusion, the anti-PAD4 response in RA patients involves all three isotypes in RA. Anti-PAD4 IgA and IgG are associated with erosive disease in RA and represent useful markers for patient stratification.

It is understood that modifications, which do not substantially affect the activity of the various embodiments of this disclosure, are also included within the definition of the disclosure provided herein. Accordingly, the following examples are intended to illustrate but not limit the present disclosure.

1-55. (canceled)

56. A detection kit comprising:
(a) a peptidyl arginine deiminase 4 (PAD4) or antigenic fragment thereof;
(b) a detection probe comprising:
(i) an antibody or functional fragment thereof capable of binding to anti-PAD4 IgA; or
(ii) an antibody or functional fragment thereof capable of binding to anti-PAD4 IgG; and
(c) a solid support;
wherein the detection kit is usable in a method for diagnosing or assessing disease severity in a subject having rheumatoid arthritis (RA), and wherein a severity of the RA is based on presence of joint erosion or severe joint erosion in the subject.

57. The detection kit of claim 56, wherein the detection probe comprises the antibody or functional fragment thereof capable of binding to anti-PAD4 IgA.

58. The detection kit of claim 56, wherein the detection probe comprises the antibody or functional fragment thereof capable of binding to anti-PAD4 IgG.

59. The detection kit of claim 56, further comprising a peptidyl arginine deiminase 1 (PAD1) or antigenic fragment thereof; a peptidyl arginine deiminase 2 (PAD2) or antigenic fragment thereof; a peptidyl arginine deiminase 3 (PAD3) or antigenic fragment thereof; a peptidyl arginine deiminase 6 (PAD6) or antigenic fragment thereof; or combinations thereof.

60. The detection kit of claim 56, wherein the detection probe further comprises a reporter tag.

61. The detection kit of claim 60, wherein the reporter tag is selected from the group consisting of a fluorophore, an enzyme, a chemiluminescent moiety, a radioactive moiety, an organic dye, and an organic dye.

62. The detection kit of claim 56, wherein the solid support is selected from the group consisting of a bead, a sphere, a particle, a membrane, a chip, a slide, a plate, a well, and a test tube.

63. The detection kit of claim 56, wherein the solid support is a bead, a sphere, or a particle, and wherein the bead, the sphere, or the particle has micrometer or nanometer dimensions.

64. The detection kit of claim 56, wherein the PAD4 or antigenic fragment thereof is conjugated to the solid support.

65. The detection kit of claim 56, wherein the detection kit is configured for use with a biological sample, the biological sample selected from blood, plasma, serum, sputum, or saliva.

66. The detection kit of claim 56, further comprising a positive control selected from the group consisting of a positive control comprising anti-PAD 4 IgA and a positive control comprising anti-PAD 4 IgG.

67. The detection kit of claim 56, further comprising:
one or more ancillary reagents selected from the group consisting of immobilization buffers, incubation buffers, washing buffers, dilution buffers, detection buffers, assay buffers, and blocking buffers.

68. The detection kit of claim 56, wherein the antibody or functional fragment thereof capable of binding to anti-PAD4 IgA comprises an anti-human IgA or fragment thereof.

69. The detection kit of claim 56, wherein the antibody or functional fragment thereof capable of binding to anti-PAD4 IgG comprises an anti-human IgG or fragment thereof.

70. A method of diagnosing or assessing severity of rheumatoid arthritis (RA) in a subject suspected of having RA, the severity being based on presence of joint erosion or severe joint erosion in the subject, the method comprising:
(a) contacting a biological sample from the subject with a peptidyl arginine deiminase 4 (PAD4) or antigenic fragment thereof;
(b) contacting the biological sample with a detection probe, wherein the detection probe comprises:
(i) an antibody or functional fragment thereof capable of binding to anti-PAD4 IgA; or
(ii) an antibody or functional fragment thereof capable of binding to anti-PAD4 IgG;
(c) detecting a presence of the detection probe, and
(d) assessing RA severity in the subject based on the detecting the presence of the detection probe.

71. The method of claim 70, wherein the biological sample is selected from blood, plasma, serum, sputum, or saliva.

72. The method of claim 70, wherein the detection probe further comprises a reporter tag, and wherein detecting the presence of the detection probe comprises detecting the presence of the reporter tag of the detection probe.

73. The method of claim 71, wherein the detection probe comprises the antibody or functional fragment thereof capable of binding to anti-PAD4 IgA.

74. The method of claim 71, wherein detection probe comprises the antibody or functional fragment thereof capable of binding to anti-PAD4 IgG.

75. The method of claim 71, further comprising contacting the biological sample with a peptidyl arginine deiminase 1 (PAD1) or antigenic fragment thereof; a peptidyl arginine deiminase 2 (PAD2) or antigenic fragment thereof; a peptidyl arginine deiminase 3 (PAD3) or antigenic fragment thereof; a peptidyl arginine deiminase 6 (PAD6) or antigenic fragment thereof; or combinations thereof.
